SEQUENCE LISTING (12 sequences)

SEQ ID NO: 1 - DNA, 20 bp, Artificial Sequence
  Single strand DNA oligonucleotide
aagaccctggtcctgatcct

SEQ ID NO: 2 - DNA, 20 bp, Artificial Sequence
  Single strand DNA oligonucleotide
tgtcgcaggtccttactcct

SEQ ID NO: 3 - DNA, 19 bp, Artificial Sequence
  Single strand DNA oligonucleotide
atcatgtgggtcctctcag

SEQ ID NO: 4 - DNA, 20 bp, Artificial Sequence
  Single strand DNA oligonucleotide
gattccggaaaagaggaagg

SEQ ID NO: 5 - DNA, 20 bp, Artificial sequence
  Single strand DNA oligonucleotide
gaggccgacttcttgtatgc

SEQ ID NO: 6 - DNA, 20 bp, Artificial sequence
  Single strand DNA oligonucleotide
cggttaacccgggtaagaat

SEQ ID NO: 7 - DNA, 20 bp, Artificial sequence
  Single strand DNA oligonucleotide
gaagaggctcctggtttgtg

SEQ ID NO: 8 - DNA, 20 bp, Artificial sequence
  Single strand DNA oligonucleotide
tctcatggaagtgtgggtca

SEQ ID NO: 9 - DNA, 20 bp, Artificial sequence
  Single strand DNA oligonucleotide
tggagagacactgccaactg

SEQ ID NO: 10 - DNA, 20 bp, Artificial sequence
  Single strand DNA oligonucleotide
ttagggctgtgtacgtgctg

SEQ ID NO: 11 - DNA, 20 bp, Artificial sequence
  Single strand DNA oligonucleotide
ggatcaaccccatacaccag

SEQ ID NO: 12 - DNA, 20 bp, Artificial sequence
  Single strand DNA oligonucleotide
tggtttgaccacggtgacta

(19) United States
(12) Patent Application Publication
(10) Pub. No.: US 2024/0041902 A1
(43) Pub. Date: Feb. 8, 2024

(54) COMPOSITIONS AND METHODS FOR THE TREATMENT OF UROTHELIAL CANCER

(71) Applicants: The State of Israel, Ministry of Agriculture & Rural Development, Agricultural Research Organization, Rishon-LeZion (IL); Tel HaShomer Medical Research Infrastructure and Services Ltd., Ramat-Gan (IL)

(72) Inventors: Hinanit KOLTAI, Rishon-LeZion (IL); Omer ANIS, Kiryat Ono (IL)

(73) Assignees: The State of Israel, Ministry of Agriculture & Rural Development, Agricultural Research Organization, Rishon-LeZion (IL); Tel HaShomer Medical Research Infrastructure and Services Ltd., Ramat-Gan (IL)

(21) Appl. No.: 18/266,029

(22) PCT Filed: Dec. 8, 2021
(86) PCT No.: PCT/IL2021/051462

§ 371 Date: Jun. 8, 2023

Related U.S. Application Data
(60) Provisional application No. 63/122,522, filed on Dec. 8, 2020.

(51) Int. Cl. A61K 31/00; A61K 31/407; A61P 35/00

(52) U.S. Cl. CPC A61K 31/658; A61K 31/407; A61P 35/00

(57) ABSTRACT

Compositions capable of reducing viability, initiating apoptosis, inducing cell cycle arrest and/or reducing migration of urothelial carcinoma cells and methods of use thereof are provided. The compositions and uses thereof comprise cannabinoids.

### RELATED APPLICATION/S

This application claims the benefit of priority of U.S. Provisional Patent Application No. 63/122,522 filed on 8 Dec. 2020, the contents of which are incorporated herein by reference in their entirety.

### SEQUENCE LISTING STATEMENT

The ASCII file, entitled 90422 Sequence Listing.txt, created on 8 Dec. 2021, comprising 4,096 bytes, submitted concurrently with the filing of this application is incorporated herein by reference.

### FIELD AND BACKGROUND OF THE INVENTION

The present invention, in some embodiments thereof, relates to compositions and methods for the treatment of urothelial cancer.

Urothelial cancer is the most common disease of urogenital cancer after prostate cancer. Urothelial cancers encompass carcinomas of the bladder, kidney and accessory organs (e.g. ureter, urethra, renal pelvis). It originates from a transitional epithelium tissue lining the inner surface of these hollow organs. Disease progression and reduction of disease recurrence are the main goals of treatment, thus new efficient therapies with acceptable side-affect profile are still needed.

Marijuana (Cannabis sativa) contains more than 500 constituents, among them more than a hundred terpenophenolic compounds termed phytocannabinoids [ElSohly et al., Phytochemistry of Cannabis sativa L. Phytocannabinoids, Springer (2017) 1-36]. An increasing number of studies have shown that phytocannabinoids can prevent proliferation, metastasis, angiogenesis and induce apoptosis in a variety of cancer cell types including breast, lung, prostate, skin, intestine, glioma, and others (e.g. International Patent Application Publication Nos: WO2016097831, WO2018/163164 and WO2020/121312, EP Patent No: EP1071417, U.S. Pat. Nos. 8,632,825 and 8,632,825). However, there is only scarce evidence on the effect of cannabis on urothelial carcinoma growth. A large epidemiologic study showed that cannabis use resulted in decreased risk of UC [Thomas A. A. et al. Urology (2015) 85, 388-393]. Activation of cannabinoid receptor type 2 (CB2) was shown to affect UC cell viability, induce apoptosis and caspase 3-activation, reduce cell motility and affect sphingolipids metabolism [Bettiga A. et al. Sci. Rep. (2017) 7, 1-11].

Additional background art includes:
International Patent Application Publication No. WO2018/163163.

### SUMMARY OF THE INVENTION

According to an aspect of some embodiments of the present invention there is provided a composition capable of reducing viability, inducing cell cycle arrest and/or reducing migration of cancer cells selected from the group consisting of:
(i) a composition comprising cannabinoids, wherein at least 80% of the cannabinoids comprise cannabichromene (CBC) or cannabichromene (CBC) and tetrahydrocannabinol (THC); and
(ii) a composition comprising cannabinoids, wherein at least 50% of the cannabinoids comprise cannabidiol (CBD) and/or tetrahydrocannabinol (THC), and at least one cannabinoid selected from the group consisting of cannabidivarinic acid (CBDVA), cannabigerol (CBG), tetrahydrocannabivarin (THCV), cannabinol (CBN), cannabichromene (CBC), cannabidiolic acid (CBDA), tetrahydrocannabinolic acid (THCA) and cannabidivarin (CBDV),
for use in treating cancer in a subject in need thereof.

According to an aspect of some embodiments of the present invention there is provided a method of treating cancer in a subject in need thereof, the method comprising administering to the subject a therapeutically effective amount of a composition capable of reducing viability, inducing cell cycle arrest and/or reducing migration of cancer cells selected from the group consisting of:
(i) a composition comprising cannabinoids, wherein at least 80% of the cannabinoids comprise cannabichromene (CBC) or cannabichromene (CBC) and tetrahydrocannabinol (THC); and
(ii) a composition comprising cannabinoids, wherein at least 50% of the cannabinoids comprise cannabidiol (CBD) and/or tetrahydrocannabinol (THC), and at least one cannabinoid selected from the group consisting of cannabidivarinic acid (CBDVA), cannabigerol (CBG), tetrahydrocannabivarin (THCV), cannabinol (CBN), cannabichromene (CBC), cannabidiolic acid (CBDA), tetrahydrocannabinolic acid (THCA) and cannabidivarin (CBDV),
thereby treating the cancer in the subject.

According to an aspect of some embodiments of the present invention there is provided a method of reducing viability, inducing cell cycle arrest and/or reducing migration of a cancerous cell, the method comprising contacting the cancerous cell with a composition capable of reducing viability, inducing cell cycle arrest and/or reducing migration of cancer cells selected from the group consisting of:
(i) a composition comprising cannabinoids, wherein at least 80% of the cannabinoids comprise cannabichromene (CBC) or at least 80% cannabichromene (CBC) and tetrahydrocannabinol (THC); and
(ii) a composition comprising cannabinoids, wherein at least 50% of the cannabinoids comprise cannabidiol (CBD) and/or tetrahydrocannabinol (THC), and at least one cannabinoid selected from the group consisting of cannabidivarinic acid (CBDVA), cannabigerol (CBG), tetrahydrocannabivarin (THCV), cannabinol (CBN), cannabichromene (CBC), cannabidiolic acid (CBDA), tetrahydrocannabinolic acid (THCA) and cannabidivarin (CBDV).

According to some embodiments of the invention, the composition (i) comprises the CBC and the THC, wherein the composition comprises in the cannabinoids thereof at least 5% of the CBC and at least 5% of the THC.

According to an aspect of some embodiments of the present invention there is provided a composition capable of reducing viability, inducing cell cycle arrest and/or reducing migration of cancer cells selected from the group consisting of:
(i) a composition comprising cannabinoids, wherein at least 80% of the cannabinoids comprise cannabichromene (CBC) and tetrahydrocannabinol (THC), wherein the composition comprises in the cannabinoids thereof at least 5% of the CBC and at least 5% of the THC; and
(ii) a composition comprising cannabinoids listed in the GB10011-F4 composition of Table 1 in percentages as listed in the GB10011-F4 composition of Table 1±10%, cannabinoids listed in the GB10011-F5 composition of Table 1 in percentages as listed in the GB10011-F5 composition of Table 1±10%, or cannabinoids listed in the GB10011-F6 composition of Table 1 in percentages as listed in the GB10011-F6 composition of Table 1±10%.

According to some embodiments of the invention, the at least 80% in the composition (i) is at least 90%.

According to some embodiments of the invention, the at least 80% in the composition (i) is at least 95%.

According to some embodiments of the invention, the composition (i) comprises in the cannabinoids at least 10% of the CBC.

According to some embodiments of the invention, the composition (i) comprises in the cannabinoids at least 10% of the THC.

According to some embodiments of the invention, the composition (i) is devoid of a cannabinoid selected from the group consisting of cannabidiol (CBD), cannabigerol (CBG) and tetrahydrocannabinolic acid (THCA).

According to some embodiments of the invention, the composition (i) is devoid of cannabinoids other than the CBC and THC.

According to some embodiments of the invention, the composition (i) comprises the CBC and the THC in a concentration ratio of 15:1-1:15.

According to some embodiments of the invention, the composition (i) comprises the CBC and the THC in a concentration ratio of 10:1-1:5.

According to some embodiments of the invention, the composition (i) comprises in the cannabinoids 80-85% CBC and 10-20% THC.

According to some embodiments of the invention, the composition (i) comprises in the cannabinoids 20-30% CBC and 70-80% THC.

According to some embodiments of the invention, the composition (i) has a combined synergistic cytotoxic activity on urothelial carcinoma cells as compared to each of the CBC and THC when administered as a single agent.

According to some embodiments of the invention, the at least one cannabinoid in the cannabinoids of composition (ii) comprises at least two cannabinoids.

According to some embodiments of the invention, the at least 50% in the cannabinoids of composition (ii) is at least 60%.

According to some embodiments of the invention, the at least 50% in the cannabinoids of composition (ii) is at least 90%.

According to an aspect of some embodiments of the present invention there is provided a composition capable of reducing viability, inducing cell cycle arrest and/or reducing migration of urothelial carcinoma cells selected from the group consisting of:
(i) a composition comprising at least 80% cannabichromene (CBC) and optionally tetrahydrocannabinol (THC); and
(ii) a composition comprising at least 50% cannabidiol (CBD) and/or tetrahydrocannabinol (THC), and at least one cannabinoid selected from the group consisting of cannabidivarinic acid (CBDVA), cannabigerol (CBG), tetrahydrocannabivarin (THCV), cannabinol (CBN), cannabichromene (CBC), cannabidiolic acid (CBDA), tetrahydrocannabinolic acid (THCA) and cannabidivarin (CBDV),
for use in treating cancer in a subject in need thereof.

According to an aspect of some embodiments of the present invention there is provided a method of treating cancer in a subject in need thereof, the method comprising administering to the subject a therapeutically effective amount of a composition capable of reducing viability, inducing cell cycle arrest and/or reducing migration of urothelial carcinoma cells selected from the group consisting of:
(i) a composition comprising at least 80% cannabichromene (CBC) and optionally tetrahydrocannabinol (THC); and
(ii) a composition comprising at least 50% cannabidiol (CBD) and/or tetrahydrocannabinol (THC), and at least one cannabinoid selected from the group consisting of cannabidivarinic acid (CBDVA), cannabigerol (CBG), tetrahydrocannabivarin (THCV), cannabinol (CBN), cannabichromene (CBC), cannabidiolic acid (CBDA), tetrahydrocannabinolic acid (THCA) and cannabidivarin (CBDV),
thereby treating the cancer in the subject.

According to an aspect of some embodiments of the present invention there is provided a method of reducing viability, inducing cell cycle arrest and/or reducing migration of a cancerous cell, the method comprising contacting the cancerous cell with a composition capable of reducing viability, inducing cell cycle arrest and/or reducing migration of urothelial carcinoma cells selected from the group consisting of:
(i) a composition comprising at least 80% cannabichromene (CBC) and optionally tetrahydrocannabinol (THC); and
(ii) a composition comprising at least 50% cannabidiol (CBD) and/or tetrahydrocannabinol (THC), and at least one cannabinoid selected from the group consisting of cannabidivarinic acid (CBDVA), cannabigerol (CBG), tetrahydrocannabivarin (THCV), cannabinol (CBN), cannabichromene (CBC), cannabidiolic acid (CBDA), tetrahydrocannabinolic acid (THCA) and cannabidivarin (CBDV).

According to some embodiments of the invention, the composition (i) comprises the CBC and the THC, wherein the composition comprises in said cannabioinds thereof at least 5% of the CBC and at least 5% of the THC.

According to an aspect of some embodiments of the present invention there is provided a composition capable of reducing viability, inducing cell cycle arrest and/or reducing migration of urothelial carcinoma cells selected from the group consisting of:
(i) a composition comprising at least 80% cannabichromene (CBC) and tetrahydrocannabinol (THC), wherein the composition comprises in said cannabioinds thereof at least 5% of the CBC and at least 5% of the THC; and
(ii) a composition comprising cannabinoids listed in the GB10011-F4 composition of Table 1 in percentages as listed in the GB10011-F4 composition of Table 1±10%, cannabinoids listed in the GB10011-F5 composition of Table 1 in percentages as listed in the GB10011-F5 composition of Table 1±10%, or cannabinoids listed in the GB10011-F6 composition of Table 1 in percentages as listed in the GB10011-F6 composition of Table 1±10%.

According to an aspect of some embodiments of the present invention there is provided a composition capable of reducing viability, inducing cell cycle arrest and/or reducing migration of cancer cells selected from the group consisting of:
(i) a composition comprising at least 80% cannabichromene (CBC) and tetrahydrocannabinol (THC), wherein the composition comprises in said cannabioinds thereof at least 5% of the CBC and at least 5% of the THC; and
(ii) a composition comprising cannabinoids listed in the GB10011-F4 composition of Table 1 in percentages as listed in the GB10011-F4 composition of Table 1±10%, cannabinoids listed in the GB10011-F5 composition of Table 1 in percentages as listed in the GB10011-F5 composition of Table 1±10%, or cannabinoids listed in the GB10011-F6 composition of Table 1 in percentages as listed in the GB10011-F6 composition of Table 1±10%.

According to some embodiments of the invention, the at least 80% is the composition (i) is at least 90%.

According to some embodiments of the invention, the at least 80% in the composition (i) is at least 95%.

According to some embodiments of the invention, the composition (i) comprises at least 10% of the CBC.

According to some embodiments of the invention, the composition (i) comprises at least 10% of the THC.

According to some embodiments of the invention, the composition (i) is devoid of a cannabinoid selected from the group consisting of cannabidiol (CBD), cannabigerol (CBG) and tetrahydrocannabinolic acid (THCA).

According to some embodiments of the invention, the composition (i) is devoid of cannabinoids other than the CBC and THC.

According to some embodiments of the invention, the composition (i) comprises at least 7 μg/ml CBC.

According to some embodiments of the invention, the composition (i) comprises at least 11 μg/ml CBC.

According to some embodiments of the invention, the composition (i) comprises at least 1 μg/ml CBC.

According to some embodiments of the invention, the composition (i) comprises at least 2 μg/ml THC.

According to some embodiments of the invention, the composition (i) comprises at least 1 μg/ml THC.

According to some embodiments of the invention, the composition (i) comprises at least 3 μg/ml THC.

According to some embodiments of the invention, the composition (i) comprises at least 5 μg/ml THC.

According to some embodiments of the invention, the composition (i) comprises at least 13 μg/ml THC.

According to some embodiments of the invention, the composition (i) comprises the CBC and the THC in a concentration ratio of 15:1-1:15.

According to some embodiments of the invention, the composition (i) comprises the CBC and the THC in a concentration ratio of 10:1-1:5.

According to some embodiments of the invention, the composition (i) comprises the CBC and the THC in a concentration ratio of 5:1-1:2.

According to some embodiments of the invention, the composition (i) comprises the CBC and the THC in a concentration ratio of about 6:1.

According to some embodiments of the invention, the composition (i) comprises the CBC and the THC in a concentration and a concentration ratio according to Table 2±10%.

According to some embodiments of the invention, the composition (i) comprises the CBC and the THC in a ratio of about 6:1 and comprises at least 11 μg/ml CBC and at least 2 μg/ml THC.

According to some embodiments of the invention, the composition (i) comprises 80-85% CBC and 10-20% THC.

According to some embodiments of the invention, the composition (i) comprises 20-30% CBC and 70-80% THC.

According to some embodiments of the invention, the composition (i) comprises about 10-15 μg/ml CBC and about 1-5 μg/ml THC.

According to some embodiments of the invention, the composition (i) comprises about 7-12 μg/ml CBC and about 5-13 μg/ml THC.

According to some embodiments of the invention, the composition (i) has a combined synergistic cytotoxic activity on urothelial carcinoma cells as compared to each of the CBC and THC when administered as a single agent.

According to some embodiments of the invention, the at least one cannabinoid in composition (ii) comprises at least two cannabinoids.

According to some embodiments of the invention, the at least 50% in the composition (ii) is at least 60%.

According to some embodiments of the invention, the at least 50% in the composition (ii) is at least 80%.

According to some embodiments of the invention, the at least 50% in the composition (ii) is at least 90%.

According to some embodiments of the invention, the CBD is the most abundant cannabinoid in the composition (ii).

According to some embodiments of the invention, the composition (ii) comprises the cannabinoids listed in the crude extract composition of Table 1.

According to some embodiments of the invention, the composition (ii) comprises percentages of cannabinoids as listed in the crude extract composition of Table 1±10%.

According to some embodiments of the invention, the composition (ii) comprises the cannabinoids listed in the GB10011-F4 composition of Table 1.

According to some embodiments of the invention, the composition (ii) comprises percentages of cannabinoids as listed in the GB10011-F4 composition of Table 1±10%.

According to some embodiments of the invention, the composition (ii) comprises the cannabinoids listed in the sCBD crude extract composition of Table 6, and wherein the cancer is urothelial cancer.

According to some embodiments of the invention, the composition (ii) comprises percentages of cannabinoids as listed in the sCBD crude extract composition of Table 6±10%, and wherein the cancer is urothelial cancer.

According to some embodiments of the invention, the composition (ii) comprises the cannabinoids listed in the PARIS crude extract composition of Table 6, and wherein the cancer is urothelial cancer.

According to some embodiments of the invention, the composition (ii) comprises percentages of cannabinoids as listed in the PARIS crude extract composition of Table 6±10%, and wherein the cancer is urothelial cancer.

According to some embodiments of the invention, the THC is the most abundant cannabinoid in the composition (ii).

According to some embodiments of the invention, the composition (ii) comprises the cannabinoids listed in the GB10011-F5 composition of Table 1.

According to some embodiments of the invention, the composition (ii) comprises percentages of cannabinoids as listed in the GB10011-F5 composition of Table 1±10%.

According to some embodiments of the invention, the composition (ii) comprises the cannabinoids listed in the GB10011-F6 composition of Table 1.

According to some embodiments of the invention, the composition (ii) comprises % of cannabinoids as listed in the GB10011-F6 composition of Table 1±10%.

According to some embodiments of the invention, the composition (ii) comprises the cannabinoids listed in the DQ crude extract composition of Table 6, and wherein the cancer is urothelial cancer.

According to some embodiments of the invention, the composition (ii) comprises percentages of cannabinoids as listed in the DQ crude extract composition of Table 6±10%, and wherein the cancer is urothelial cancer.

According to some embodiments of the invention, the composition (ii) comprises the cannabinoids listed in the DQ F7 composition of Table 6, and wherein the cancer is urothelial.

According to some embodiments of the invention, the composition (ii) comprises percentages of cannabinoids as listed in the DQ F7 composition of Table 6±10%, and wherein the cancer is urothelial cancer.

According to some embodiments of the invention, the composition is a liquid chromatography fraction of a cannabis extract.

According to some embodiments of the invention, the liquid chromatography fraction is obtainable by subjecting the cannabis extract to flash chromatography comprising a Flash chromatography apparatus equipped with a diode array detector, a C18 functionalized silica column, a 55% to 100% water and methanol gradient at a flow rate of 30 ml/min.

According to some embodiments of the invention, the composition (i) is collected between about 22-22.5 minutes of the flash chromatography.

According to some embodiments of the invention, the composition (ii) is collected between about 15-17 minutes, 17-20 minutes, or 20-22 minutes of the flash chromatography.

According to some embodiments of the invention, the cannabinoids are purified from cannabis.

According to some embodiments of the invention, the cannabis is a cannabis strain IGB chemovar 10011.

According to some embodiments of the invention, the composition is a synthetic composition.

According to some embodiments of the invention, the composition (i) is a synthetic composition consisting of the CBC and the THC as the cannabinoids.

According to some embodiments of the invention, presence or absence of the cannabinoids in the composition is effected by high pressure liquid chromatography (HPLC).

According to some embodiments of the invention, the composition is capable of inducing apoptosis of urothelial carcinoma cells.

According to some embodiments of the invention, the composition is capable of inducing necrosis of urothelial carcinoma cells.

According to some embodiments of the invention, the composition is capable of inducing apoptosis of cancer cells.

According to some embodiments of the invention, the composition is capable of inducing necrosis of cancer cells.

According to some embodiments of the invention, the composition is capable of inhibiting sphere formation and/or epithelial to mesenchymal transition of urothelial carcinoma cells.

According to some embodiments of the invention, the cancer is selected from the group consisting of urothelial cancer, uterine cervix carcinoma, prostate cancer and melanoma.

According to some embodiments of the invention, the cancer is urothelial cancer.

According to some embodiments of the invention, the composition for use further comprising an anti-cancer agent.

According to some embodiments of the invention, the method further comprising administering to the subject a therapeutically effective amount of an anti-cancer agent.

According to some embodiments of the invention, the method further comprising contacting the cancerous cell with an anti-cancer agent.

According to an aspect of some embodiments of the present invention there is provided an article of manufacture comprising the composition and an anti-cancer agent.

According to some embodiments of the invention, the composition and the anti-caner agent are in a co-formulation.

According to some embodiments of the invention, the composition and the anti-cancer agent are in separate formulations.

According to some embodiments of the invention, the anti-cancer agent is selected from the group consisting of Mitomycin C, Cisplatinum, carboplatinum, MVAC (methotrexate, vinblastine, Adriamycin, cisplatin), gemzar and Cisplatin-Gemzar.

According to some embodiments of the invention, the anti-cancer agent is Mitomycin C.

According to some embodiments of the invention, the anti-cancer agent is an immune-modulatory agent.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A shows a flash chromatography profile of crude extract of C. sativa IGB strain 10011. Fractions were collected and designated GB10011-F4-GB10011-F8.

FIG. 1B demonstrates cell viability of T24 cells treated with the crude extract or different fractions of the extract obtained from C. sativa IGB strain. Cell viability was determined by XTT assay as a function of live cell number. Cells were treated with the crude extract and fractions GB10011-F4-GB10011-F8 at a concentration of 30 μg/mL for 48 h. Mitomycin-C (MMC, 8 μg/mL) served as a positive control. Methanol treatment served as a solvent (vehicle) control. Error bars indicate ±SE (n=3). Levels with different letters are significantly different from all combinations of pairs by Tukey-Kramer honest significant difference (HSD; P≤0.05).

FIGS. 1C-G shows dose-effect curves of the crude extract, fractions GB10011-F4, GB10011-F5, GB10011-F6, GB10011-F7 and a synthetic composition comprising CBC+THC (11.5+2.0 μg/mL, respectively) on the viability of the T24 cell line. Data points were connected by non-linear regression lines of the sigmoidal dose-response relation. GraphPad Prism was used to produce the dose-response curve and IC50 doses. The concentrations used for the dose-effect curves were 10%, 30%, 50%, 75%, 100%, 125% and 150% of the respective composition.

FIG. 1H shows a dose-effect curve of a synthetic composition comprising CBC+THC on the viability of HTB-9 cell line. Data points were connected by non-linear regression lines of the sigmoidal dose-response relation. GraphPad Prism was used to produce the dose-response curve and IC50 doses. The concentrations used for the dose-effect curve were 10%, 30%, 50%, 75%, 100%, 125% 150% and 175% of the original CBC+THC composition containing 11.5+2.0 μg/mL, respectively.

FIG. 2 demonstrates synergistic cytotoxic activity of combined treatment with CBC and THC, as determined by the effect on viability of T24 cells. Shown are percentages of cell viability following treatment with CBC and THC at the indicated concentrations.

FIGS. 3A-B demonstrate the involvement of the cannabinoid receptors in the cytotoxic activity against UCC. FIG. 3A shows viability of T24 cells treated with CBC+THC (11.5+2.0 μg/mL, respectively) with or without a CB1 or CB2 inverse agonist (IA), a TRPV1 or TRPV2 antagonist or a TRPA1 blocker (10 μM), as determined by XTT assay. Treatment with Mitomycin-C (MMC, 8 μg/mL) served as a positive control. Methanol (control) treated served as a vehicle control. Error bars indicate ±SE (n=3). Levels with different letters are significantly different from all combinations of pairs by Tukey-Kramer honest significant difference (HSD). FIG. 3B shows RNA levels of CB1 receptor (CNR1) or CB2 receptor (CNR2) in T24 cells following 6 hours treatment with CBC+THC (11.2+1.8 μg/mL, respectively) or CBD (9.2 μg/mL) relative to control. Methanol (control) treatment served as a vehicle control. Gene transcript values were determined by quantitative PCR as a ratio between the target gene versus a reference gene (HPRT1; geneID 3251). Values were calculated relative to the average expression of target genes in treated versus control using the 2° Act method.

FIG. 4A demonstrates the effect of 24 hours treatment with CBC+THC (17.2+2.8 μg/mL, respectively) or CBD (15 μg/mL) on cell cycle stages of T24 cells. Mitomycin-C (MMC, 8 μg/mL) served as a positive control. Methanol (control) treatment served as a vehicle control. Error bars indicate ±SE (n=3). Levels with different letters are significantly different from all combinations of pairs according to Tukey-Kramer honest significant difference (HSD; P<0.05). * indicates significantly different mean from the control based on Student t-test (P<0.05).

FIG. 4B demonstrates the effect of 48 hours treatment with CBC+THC (17.2+2.8 μg/mL, respectively) or CBD (15 μg/mL) on the proportion of viable, apoptotic and necrotic T24 cells. Mitomycin-C (MMC, 8 μg/mL) served as a positive control. Methanol (control) treatment served as a vehicle control. Error bars indicate ±SE (n=3). Levels with different letters are significantly different from all combinations of pairs according to Tukey-Kramer honest significant difference (HSD; P<0.05).

FIGS. 5A-C demonstrate the effect of treatment with CBC+THC or CBD on T24 cells migration, as determined by the scratch assay. FIG. 5A shows representative pictures taken for estimation of the effect of CBC+THC (8.6+1.4 μg/mL, respectively) or CBD (10 μg/mL) on recovered area of confluent monolayers of T24 cells at 0, 10, 12, 14 and 16 hours following treatment. FIG. 5B is a graph summing the effect on recovered area of confluent monolayers in the scratch assay. FIG. 5C shows the effect of treatment with CBC+THC (8.6+1.4 μg/mL respectively), CBC (10 μg/mL), THC (10 μg/mL) or CBC+THC with CB1 or CB2 inverse agonist (IA, 10 μM) on the recovered area of confluent monolayers of T24 cells at 14 hours. In all Figures Mitomycin-C (MMC, 4 μg/mL) served as a positive control. Methanol (control) treatment served as a vehicle control. Values are means±SE (n=12). Values with different letters are significantly different from all combinations of pairs according to Tukey-Kramer honest significant difference (HSD; P<0.05).

FIG. 6 shows confocal images of T24 cells following 24 hours treatment with CBC+THC (8.6+1.4 μg/mL, respectively) or CBD (10 μg/mL). Cell were stained for F-actin (EasyProbes™ ActinRed 555 Stain, red stain; shown on the left), and nuclei (Hoechst, blue stain; both stains shown on the right). Mitomycin-C (MMC, 4 μg/mL) served as a positive control. Methanol (control) treatment served as a vehicle control. Bars=20 μm; yellow arrows point to disintegration of F-actin filaments visualized as characteristic spots; white arrows point to induced accumulation of F-actin filaments in the cell periphery.

FIG. 7 demonstrates cell viability of HTB-9 cells treated with different fractions of C. sativa IGB strain extract and fractions. Cell viability was determined by XTT assay as a function of live cell number. Cells were treated with the crude extract and fractions GB10011-F4, GB10011-F5, GB10011-F6, GB10011-F7 and GB10011-F8 at a concentration of 30 μg/mL for 48 hours. Mitomycin-C (MMC, 8 μg/mL) served as a positive control. Methanol (control) treatment served as a solvent (vehicle) control. Error bars indicate ±SE (n=3). Levels with different letters are significantly different from all combinations of pairs by Tukey-Kramer honest significant difference (HSD; P≤0.05).

FIG. 8 shows viability of T24 cells treated with CBD (13.8 μg/mL) with or without a CB1 or CB2 inverse agonist (IA; 10 μM), as determined by XTT assay. Treatment with Mitomycin-C (MMC, 8 μg/mL) served as a positive control. Methanol (control) treated served as a vehicle control. Error bars indicate ±SE (n=3). Levels with different letters are significantly different from all combinations of pairs by Tukey-Kramer honest significant difference (HSD).

FIGS. 9A-B demonstrate the effect of CBC+THC (17.2+2.8 μg/mL, respectively) or CBD (15 μg/mL) on T24 cell cycle arrest, apoptosis and necrosis. FIG. 9A shows representative FACS histograms demonstrating PI staining for determination the stages of cell cycle arrest performed 24 hours following treatment. FIG. 9B shows FACS plots demonstrating Annexin V-FITC and PI staining for determination of proportion of viable (Q4), apoptotic (Q2 and Q3 for late and early apoptosis, respectively) or necrotic (Q1) cells performed 48 hours following treatment. Mitomycin-C (MMC, 8 μg/mL) served as a positive control. Methanol (control) treatment served as a vehicle control.

FIG. 10 demonstrates the proportion of viable, apoptotic or necrotic T24 cells following 24 hours treatment with CBC+THC (17.2+2.8 μg/mL, respectively) or CBD (15 μg/mL). Mitomycin-C (MMC, 8 μg/mL) served as a positive control. Methanol (control) treatment served as a vehicle control. Error bars indicate ±SE (n=3). Levels with different letters are significantly different from all combinations of pairs according to Tukey-Kramer honest significant difference (HSD; P<0.05).

FIG. 11A-B is a graph illustrating the results presented in Table 3 hereinbelow and demonstrating synergistic cytotoxic activity of combined treatment with CBC and THC, as determined by the effect on viability of T24 cells. Synergy was calculated based on Bliss independence drug interaction model. Synergy is apparent when the experimental (observed) value of cell survival inhibition is higher than the calculated (expected) value. Values of delta of experimental (observed) minus calculated (expected) values calculated based on Bliss model is shown in Y axis. Lighter blue represents higher positive delta between experimental inhibition fraction to the expected inhibition fraction of the two cannabinoids at different concentrations and ratios.

FIG. 12 shows dose effect curves of DQ and sCBD crude extracts on the viability of the UC cell lines T24 and HTB9, determined by XTT assay 48 hours following treatment. Values represent mean±SE of cell viability compared to vehicle control (n=3). Data points were connected by nonlinear regression lines of the sigmoidal dose-response inhibition relation. GraphPad Prism was used to produce the dose-response curve and IC50 doses.

FIG. 13 shows dose effect curves of DQ and sCBD crude extracts on the viability of the UC cell lines T24 and HTB9, determined by XTT assay 2 hours following treatment. Values represent mean±SE of cell viability compared to vehicle control (n=3). Data points were connected by nonlinear regression lines of the sigmoidal dose-response inhibition relation. GraphPad Prism was used to produce the dose-response curve and IC50 doses.

FIG. 14 shows dose effect curves of DQ crude extract, DQ fraction F7, standard mix mimicking DQ F7 and standard mix of THC and CBC only in a ratio mimicking DQ F7 on the viability of the UC cell lines T24, determined by XTT assay. Values represent mean±SE of cell viability compared to vehicle control (n=3). Data points were connected by nonlinear regression lines of the sigmoidal dose-response inhibition relation. GraphPad Prism was used to produce the dose-response curve and IC50 doses.

FIG. 15 shows synergistic interactions between CBC (85%)+THC (15%) and Mitomycin-C (MMC) on T24 cell viability, calculated using the Bliss independence drug interaction model. Delta values between the observed and expected values, calculated using the Bliss model are shown in the Y axis.

FIGS. 16A-D shows the cytotoxic activity of crude extracts of PARIS (100 μg/ml) (FIGS. 16A-B), or sCBD (100 μg/ml) (FIG. 16C) and standard cannabinoids at the pick of synergism CBC (56%)+THC (44%) (100 μg/ml) as determined in vitro on T24 cell line (i) and ex vivo on slices of UC cancer (ii). The effect of continuous-duration treatment on the UC tumor is presented as a drug-related response (%), evaluated by an uropathologist who scores tumor cell survival of the tissue. The patients were diagnosed with nonmuscle invasive bladder tumor (FIG. 16A), a muscle-invasive bladder tumor (FIGS. 16B-C), FIG. 16C i. T24 cell viability. ii. a muscle-invasive bladder tumor. FIG. 16D i. T24 cell viability. ii. a high-grade urothelial tumor in diverticuli of the bladder, invasion is not defined.

FIG. 17 shows the cytotoxic activity of sCBD crude extract as determined on UC cancer in vitro on T24 cell line (i), in vitro on T24 cell line (ii) and ex-vivo slices (iii). The effect of two-hour treatments (100 μg/ml), repeated three times on UC tumor, is presented as a drug-related response (%), evaluated by an uropathologist that scores tumor cell survival of the tissue. Patients were diagnosed with non-muscle invasive bladder tumor.

FIG. 18A shows the stages of cell cycle arrest in T24 cells following 24 hours treatment with DQ (30 μg/ml) or sCBD (30 μg/mL) crude extract. Mitomycin-C (MMC, 8 μg/mL) served as a positive control. Methanol treatment served as a vehicle control. Error bars indicate ±SE (n=4). Levels with different letters of the same font are significantly different from all combinations of pairs according to Tukey-Kramer honest significant difference (HSD; p<0.05).

FIG. 18B shows the proportion of viable, apoptotic, or necrotic T24 cells following 48 hours treatment with DQ (30 μg/ml) or sCBD (30 μg/mL) crude extract. Mitomycin-C (MMC, 8 μg/mL) served as a positive control. Methanol treatment served as a vehicle control. Error bars indicate ±SE (n=4). Levels with different letters of the same font are significantly different from all combinations of pairs according to Tukey-Kramer honest significant difference (HSD; p≤0.05).

FIG. 19 shows the effect of sCBD extract (17.88 μg/mL), DQ extract (17.99 μg/mL) or Mitomycin C (MMC, 4 μg/mL) on T24 cell migration at 0, 10, 12, 14 and 16 hours in the scratch assay. The Y axis represents the percentage of reduction in the open scratch area, as opposed to the scratch area at time 0 h. The methanol treatment served as a solvent (vehicle) control. Values are means±SE (n=12). Values with different letters of the same font are significantly different from all pairs' combinations according to the honest Tukey-Kramer significant difference (HSD; p≤0.05).

FIGS. 20A-C show the effect of sCBD extract (15 μg/mL and 18 μg/mL), DQ extract (15 μg/mL and 18 μg/mL) or Mitomycin-C (MMC, 1 μg/mL) on the formation of T24 cells' spheres at 0, 72 and 96 hours. Methanol served as vehicle control. Cells were incubated for another 24 hours to allow programmed cell death to complete. The cells were then suspended in serum free media enriched with growth factors (DMEM-F12) and seeded in light-attachment plates. The size of the spheres (FIG. 20A) and the number of spheres (FIG. 20B) were documented at all time points. The error bars indicate ±S.E. (n=3). Levels with different letters are significantly different from all pairs combinations by Tukey-Kramer honest significant difference (HSD; P<0.05).

FIG. 21 shows the effect of sCBD and DQ extracts at sub-lethal concentrations (15 μg/ml and 18 μg/ml) or Mitomycin C (MMC, 1 μg/mL) on T24 cell regeneration after sphere formation. Methanol treatment served as solvent control (vehicle). T24 cells were transferred to a normal culture plate containing culture media. The colonies of UC cells were evaluated after 36 hours (2 cycles of generation time, defined as 19 hours by the manufacturer).

FIG. 22 shows the effect of sCBD (30 μg/ml) and DQ (30 μg/ml) extracts on mRNA expression levels of E-cadherin and N-cadherin in T24 cells. Treatment was effected for 2 hours. Cell harvesting after additional 2 or 6 hours, as indicated. Mitomycin C (MMC, 8 μg/mL) served as a positive control. The methanol (control) treatment served as a vehicle control. Gene transcript values were determined by quantitative PCR as a ratio between the target gene versus a reference gene (HPRT1; geneID 3251). The values were calculated relative to the average expression of target genes in the treated versus methanol control using the 2^{ΔΔCt} method.

### DESCRIPTION OF SPECIFIC EMBODIMENTS OF THE INVENTION

The present invention, in some embodiments thereof, relates to compositions and methods for the treatment of urothelial cancer.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Marijuana (Cannabis sativa) contains more than 500 constituents, among them phytocannabinoids, terpenes and flavonoids. Cannabinoids were previously shown to prevent proliferation, metastasis, angiogenesis and induce apoptosis in a variety of cancer cell types.

The present inventor has now uncovered specific liquid chromatography fractions of cannabis inflorescence extracts and synthetic compositions mimicking same having anti-cancer activity on urothelial carcinoma cells.

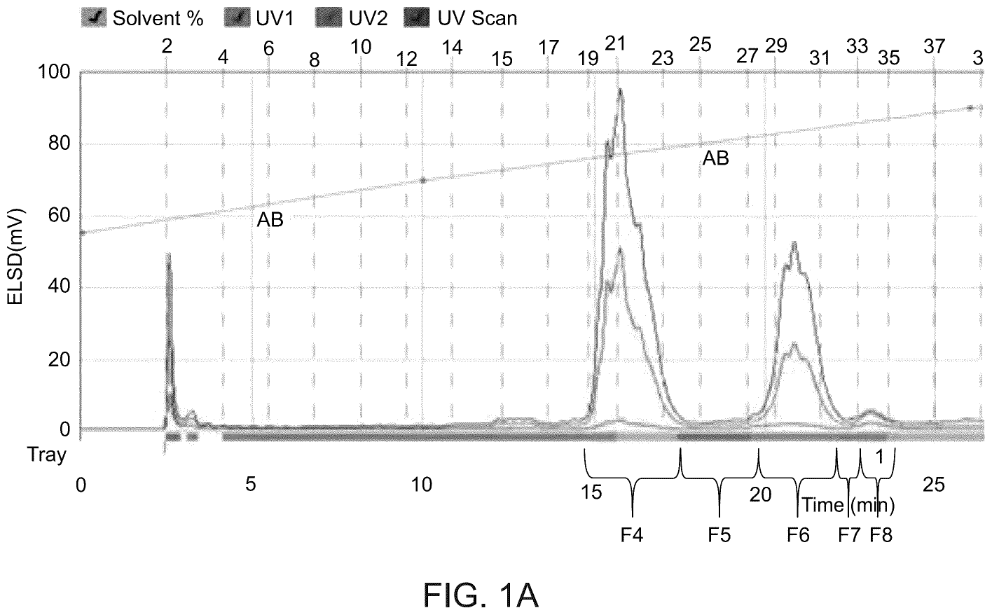

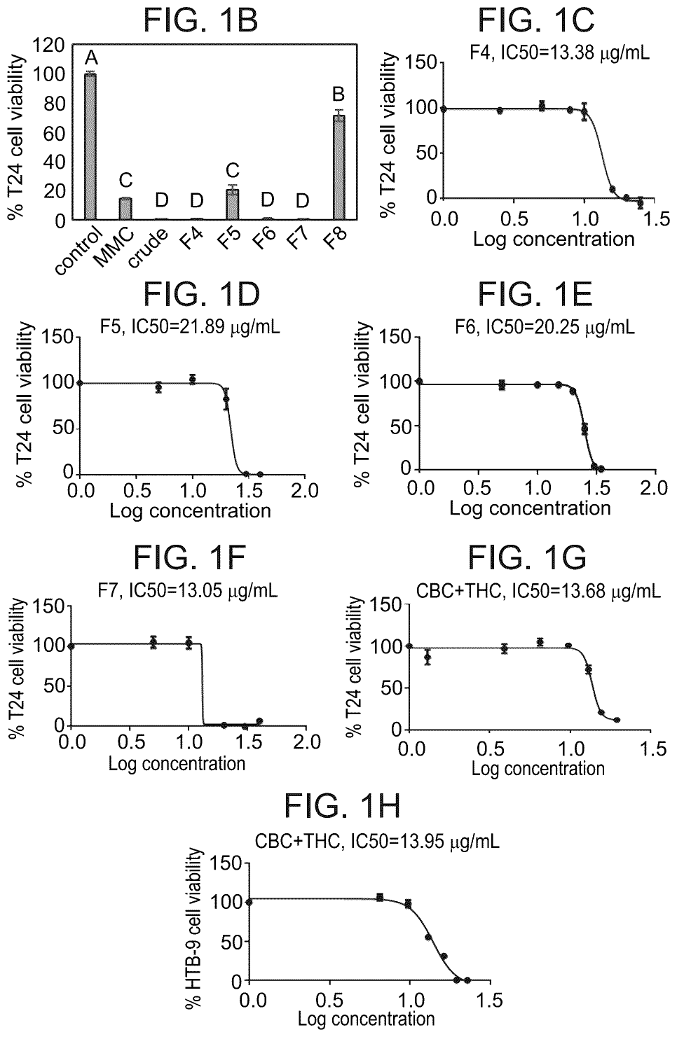

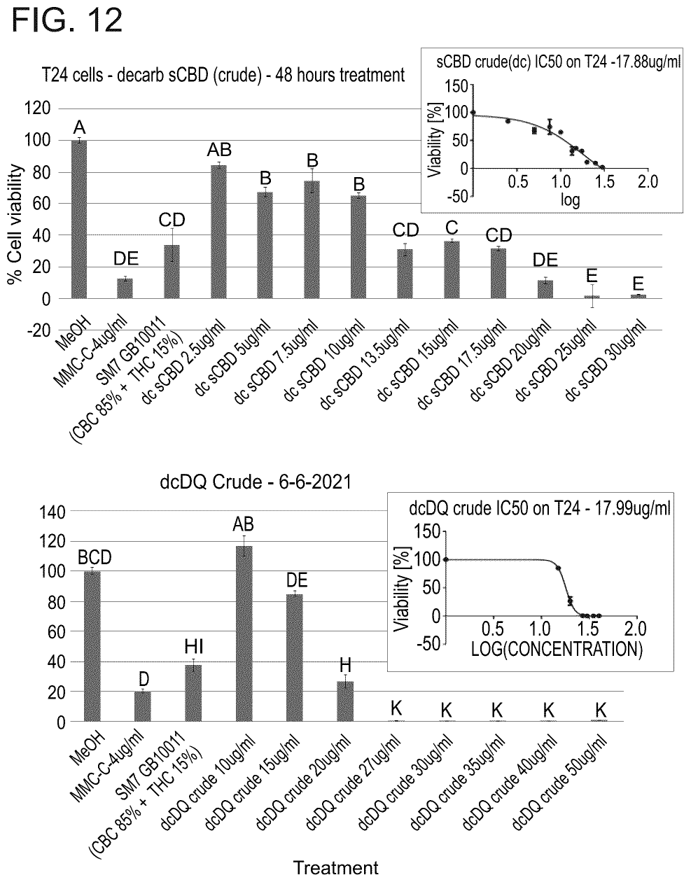

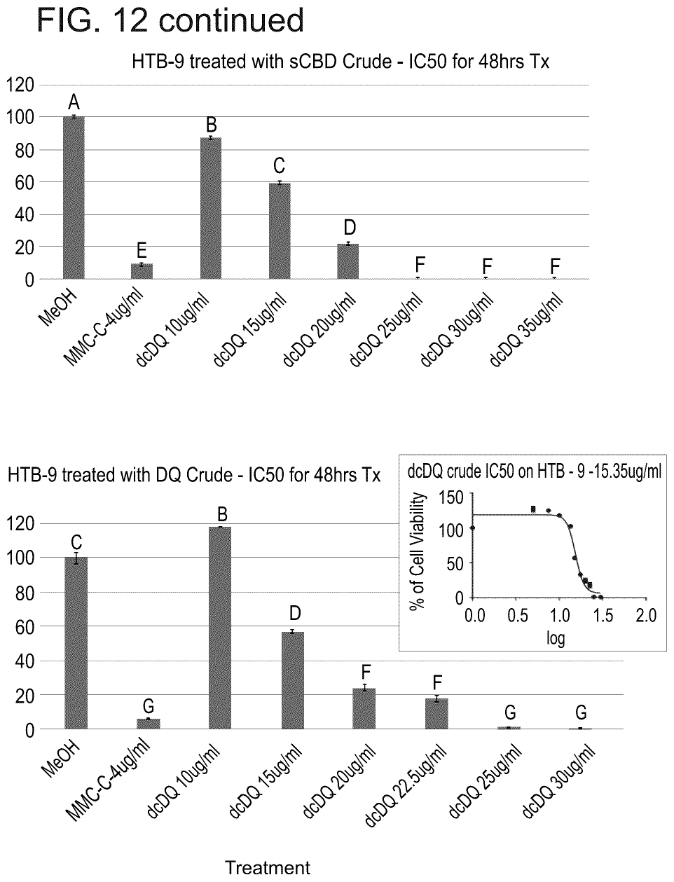

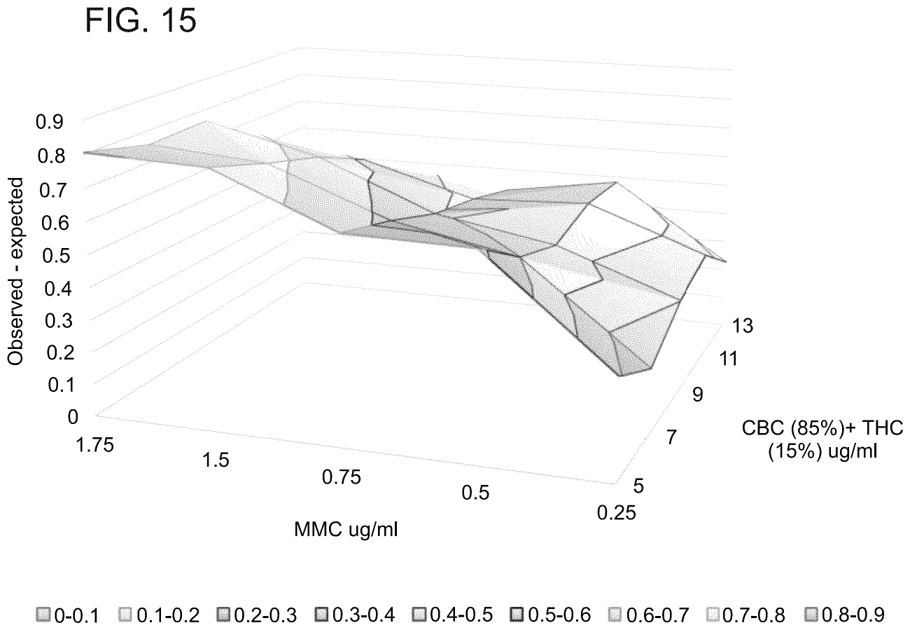

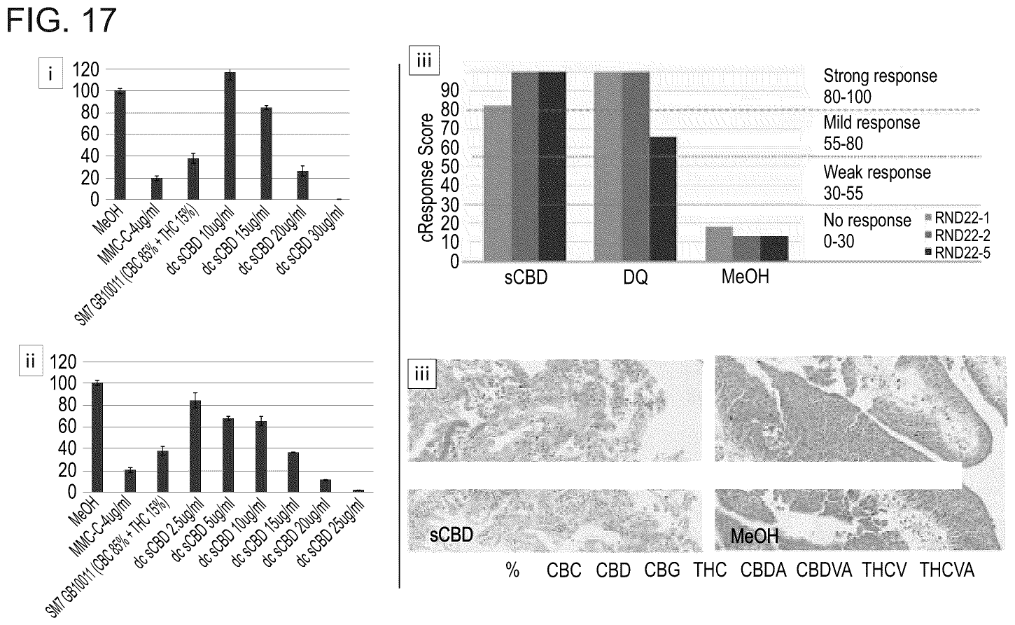

Specifically, as is illustrated in the Examples section which follows, the present inventors obtained inflorescences extracts of a high CBD strain of C. sativa IGB chemovar 10011 from the Israel Gene Bank (IGB or GB) collection and fractionated them into several distinct fractions. Following, the inventors show that the crude extracts, several fractions (referred to herein as “GB10011-F4-GB10011-F7”) and synthetic compositions mimicking same (e.g. CBC and THC at the ratios found in fraction GB10011-F7 of GB10011) have significant anti-cancer effects, manifested by reduced viability, induction of cell cycle arrest and reduced migration of urothelial carcinoma cells (Examples 1-5 of the Examples section which follows, FIGS. 1A-H, 3A-B, 4A, 5A-C, 7, 7, 9A-B, 10). This anti-cancer activity was superior compared to treatment with MMC, other fractionated fractions and the cannabinoids comprised in the fractions (e.g. CBC, THC, CBD) when administered as single compounds (Examples 1, 3 and 5 of the Example section which follows, FIGS. 1B, 2, 4A, 5A-C and 11). Importantly, the present inventor also show that a combined treatment with the obtained cannabinoid compositions and MMC had a synergistic anti-cancer effect (Example 6 of the Examples section which follows, FIG. 15). In addition, the present inventors obtained inflorescences extracts and fractions of other C. sativa strains, namely DQ, sCBD and PARIS and showed that the crude extracts, fraction DQ F7 and synthetic compositions mimicking same have significant anti-cancer effects, manifested by reduced viability, induction of cell cycle arrest, reduced migration, reduced sphere formation and epithelial to mesenchymal transformation of urothelial carcinoma cells (Examples 7-8 of the Examples section which follows, FIGS. 12-14 and 16A-22).

Consequently, specific embodiments of the present invention propose novel compositions comprising cannabinoids and their use in cancer therapy.

Thus, according to an aspect of the present invention there is provided a composition capable of reducing viability, inducing cell cycle arrest and/or reducing migration of cancer cells selected from the group consisting of:
(i) a composition comprising cannabinoids, wherein at least 80% of said cannabinoids comprise cannabichromene (CBC) and tetrahydrocannabinol (THC), wherein said composition comprises in said cannabioinds thereof at least 5% of said CBC and at least 5% of said THC; and
(ii) a composition comprising cannabinoids listed in the GB10011-F4 composition of Table 1 in percentages as listed in the GB10011-F4 composition of Table 1±10%, cannabinoids listed in the GB10011-F5 composition of Table 1 in percentages as listed in the GB10011-F5 composition of Table 1±10%, or cannabinoids listed in the GB10011-F6 composition of Table 1 in percentages as listed in the GB10011-F6 composition of Table 1±10%.

According to an additional or an alternative aspect of the present invention, there is provided a composition capable of reducing viability, inducing cell cycle arrest and/or reducing migration of urothelial carcinoma cells selected from the group consisting of:
(i) a composition comprising cannabinoids, wherein at least 80% of said cannabinoids comprise cannabichromene (CBC) and tetrahydrocannabinol (THC), wherein said composition comprises in said cannabioinds thereof at least 5% of said CBC and at least 5% of said THC; and
(ii) a composition comprising cannabinoids listed in the GB10011-F4 composition of Table 1 in percentages as listed in the GB10011-F4 composition of Table 1±10%, cannabinoids listed in the GB10011-F5 composition of Table 1 in percentages as listed in the GB10011-F5 composition of Table 1±10%, or cannabinoids listed in the GB10011-F6 composition of Table 1 in percentages as listed in the GB10011-F6 composition of Table 1±10%.

According to an additional or an alternative aspect of the present invention, there is provided a composition capable of reducing viability, inducing cell cycle arrest and/or reducing migration of cancer cells selected from the group consisting of:
(i) a composition comprising cannabinoids, wherein at least 80% of said cannabinoids comprise cannabichromene (CBC) or cannabichromene (CBC) and tetrahydrocannabinol (THC); and
(ii) a composition comprising cannabinoids, wherein at least 50% of said cannabinoids comprise cannabidiol (CBD) and/or tetrahydrocannabinol (THC), and at least one cannabinoid selected from the group consisting of cannabidivarinic acid (CBDVA), cannabigerol (CBG), tetrahydrocannabivarin (THCV), cannabinol (CBN), cannabichromene (CBC), cannabidiolic acid (CBDA), tetrahydrocannabinolic acid (THCA) and cannabidivarin (CBDV),
for use in treating cancer in a subject in need thereof.

According to an additional or an alternative aspect of the present invention there is provided a method of treating cancer in a subject in need thereof, the method comprising administering to the subject a therapeutically effective amount of a composition capable of reducing viability, inducing cell cycle arrest and/or reducing migration of cancer cells selected from the group consisting of:
(i) a composition comprising cannabinoids, wherein at least 80% of said cannabinoids comprise cannabichromene (CBC) or cannabichromene (CBC) and tetrahydrocannabinol (THC); and
(ii) a composition comprising cannabinoids, wherein at least 50% of said cannabinoids comprise cannabidiol (CBD) and/or tetrahydrocannabinol (THC), and at least one cannabinoid selected from the group consisting of cannabidivarinic acid (CBDVA), cannabigerol (CBG), tetrahydrocannabivarin (THCV), cannabinol (CBN), cannabichromene (CBC), cannabidiolic acid (CBDA), tetrahydrocannabinolic acid (THCA) and cannabidivarin (CBDV),
thereby treating the cancer in the subject.

According to an additional or an alternative aspect of the present invention, there is provided a composition capable of reducing viability, inducing cell cycle arrest and/or reducing migration of urothelial carcinoma cells selected from the group consisting of:
(i) a composition comprising at least 80% cannabichromene (CBC) and optionally tetrahydrocannabinol (THC); and
(ii) a composition comprising at least 50% cannabidiol (CBD) and/or tetrahydrocannabinol (THC), and at least one cannabinoid selected from the group consisting of cannabidivarinic acid (CBDVA), cannabigerol (CBG), tetrahydrocannabivarin (THCV), cannabinol (CBN), cannabichromene (CBC), cannabidiolic acid (CBDA), tetrahydrocannabinolic acid (THCA) and cannabidivarin (CBDV),
for use in treating cancer in a subject in need thereof.

According to an additional or an alternative aspect of the present invention, there is provided a method of treating cancer in a subject in need thereof, the method comprising administering to the subject a therapeutically effective amount of a composition capable of reducing viability, inducing cell cycle arrest and/or reducing migration of urothelial carcinoma cells selected from the group consisting of:
(i) a composition comprising at least 80% cannabichromene (CBC) and optionally tetrahydrocannabinol (THC); and
(ii) a composition comprising at least 50% cannabidiol (CBD) and/or tetrahydrocannabinol (THC), and at least one cannabinoid selected from the group consisting of cannabidivarinic acid (CBDVA), cannabigerol (CBG), tetrahydrocannabivarin (THCV), cannabinol (CBN), cannabichromene (CBC), cannabidiolic acid (CBDA), tetrahydrocannabinolic acid (THCA) and cannabidivarin (CBDV),
thereby treating the cancer in the subject.

As used herein, the term “cannabinoid” refers to a phytocannabinoid.

According to a specific embodiment, the term “phytocannabinoid” refers to a meroterpenoid with a resorcinyl core typically decorated with a para-positioned isoprenyl, alkyl, or aralkyl side chain originated from a cannabis plant, acidic or decarboxylated acid forms thereof. The term also reads on synthetic analogs or derivatives of the plant originated substance.

Alternatively or additionally, the term “phytocannabinoid” refers to a cannabinoid selected from the list provided in Table 5 hereinbelow originated from a cannabis plant, acidic or decarboxylated acid forms thereof. The term also reads on synthetic analogs or derivatives of the plant originated substance.

TABLE 5
List of phytocannabinoids (modified from Berman P, Futoran K, Lewitus GM,
Mukha D, Benami M, Shlomi T, Meiri D. A new ESI-LC/MS approach for comprehensive
metabolic profiling of phytocannabinoids in Cannabis. Scientific reports. 2018 Sep. 24; 8(1):1-5,
the contents of which are fully incorporated herein by reference)

Cannabiorcol-C1 (CBNO)
CBND-C1 (CBNDO)*
(−)-Δ 9-trans-Tetrahydrocannabiorcol-C1 (Δ9-THCO)
Cannabidiorcol-C1 (CBDO)
Cannabiorchromene-C1 (CBCO)
(−)-A 8-trans-(6aR,10aR)-Tetrahydrocannabiorcol-C1 (Δ8-THCO)
Cannabiorcyclol C1 (CBLO)
CBG-C1 (CBGO)
Cannabinol (CBN)
CBND-C2
Delta-9-tetrahydrocannabinol Δ9-THC (THC)
Cannabidiol (CBD)
Δ8-THC-C2
CBL-C2
Bisnor-cannabielsoin-C1 (CBEO)
Cannabigerol (CBG)
Cannabivarin-C3 (CBNV)
Cannabinodivarin-C3 (CBNDV)
Δ9-trans-Tetrahydrocannabivarin Δ9-THCV (THCV)
(−)-Cannabidivarin-C3 (CBDV)
(±)-Cannabichromevarin-C3 (CBCV)
(−)-Δ8-trans-THC-C3 (Δ8-THCV)
Δ7-tetrahydrocannabivarin-C3 (Δ7-THCV)
(±)-(1aS,3aR,8bR,8cR)-Cannabicyclovarin-C3 (CBLV)
2-Methyl-2-(4-methyl-2-pentenyl)-7-propyl-2H-1-benzopyran-5-ol
CBE-C2
Cannabigerovarin-C3 (CBGV)
Cannabitriol-C1 (CBTO)
Cannabinol-C4 (CBN-C4)
CBND-C4
(−)-Δ9-trans-Tetrahydrocannabinol-C4 (Δ9-THC-C4)
Cannabidiol-C4 (CBD-C4)
Cannabichromene (CBC)
(−)-trans-Δ8-THC-C4
CBL-C4
Cannabielsoin-C3 (CBEV)
CBG-C4
CBT-C2
Cannabichromanone-C3
Cannabiglendol-C3 (OH-iso-HHCV-C3)
Cannabioxepane-C5 (CBX)
Dehydrocannabifuran-C5 (DCBF)
Cannabinol-C5 (CBN)
Cannabinodiol-C5 (CBND)
Cannabifuran-C5 (CBF)
(−)-Δ9-trans-Tetrahydrocannabinol-C5 (Δ9-THC)
(−)-A8-trans-(6aR,10aR)-Tetrahydrocannabinol-C5 (A8-THC)
(±)-Cannabichromene-C5 (CBC)
(−)-Cannabidiol-C5 (CBD)
(±)-(1aS,3aR,8bR,8cR)-Cannabicyclol-C5 (CBL)
Cannabicitran-C5 (CBR)
(−)-Δ9-(6aS,10aR-cis)-Tetrahydrocannabinol-C5 ((−)-cis-Δ9-THC)
(−)-Δ7-trans-(1R,3R,6R)-Isotetrahydrocannabinol-C5 (trans-iso-Δ7-THC)
CBE-C4
Cannabigerol-C5 (CBG)
Cannabitriol-C3 (CBTV)
Cannabinol methyl ether-C5 (CBNM)
CBNDM-C5
8-OH-CBN-C5 (OH-CBN)
OH-CBND-C5 (OH-CBND)
10-Oxo-Δ6a(10a)-Tetrahydrocannabinol-C5 (OTHC)
Cannabichromanone D-C5
Cannabicoumaronone-C5 (CBCON-C5)
Cannabidiol monomethyl ether-C5 (CBDM)
Δ9-THCM-C5
(±)-3″-hydroxy-Δ4″-cannabichromene-C5
(5aS,6S,9R,9aR)-Cannabielsoin-C5 (CBE)
2-geranyl-5-hydroxy-3-n-pentyl-1,4-benzoquinone-C5
8α-Hydroxy-Δ9-Tetrahydrocannabinol-C5 (8α-OH-Δ9-THC)
8β-Hydroxy-Δ9-Tetrahydrocannabinol-C5 (8β-OH-Δ9-THC)
10α-Hydroxy-Δ8-Tetrahydrocannabinol-C5 (10α-OH-Δ8-THC)
10β-Hydroxy-Δ8-Tetrahydrocannabinol-C5 (10β-OH-Δ8-THC)
10α-hydroxy-Δ9,11-hexahydrocannabinol-C5
9β,10β-Epoxyhexahydrocannabinol-C5
OH-CBD-C5 (OH-CBD)
Cannabigerol monomethyl ether-C5 (CBGM)
Cannabichromanone-C5
CBT-C4
(±)-6,7-cis-epoxycannabigerol-C5
(±)-6,7-trans-epoxycannabigerol-C5
(−)-7-hydroxycannabichromane-C5
Cannabimovone-C5
(−)-trans-Cannabitriol-C5 ((−)-trans-CBT)
(+)-trans-Cannabitriol-C5 ((+)-trans-CBT)
(+)-cis-Cannabitriol-C5 ((+)-cis-CBT)
(−)-trans-10-Ethoxy-9-hydroxy-46a(10a)-tetrahydrocannabivarin-C3 [(−)-trans-CBT-OEt]
(−)-(6aR,9S,10S,10aR)-9,10-Dihydroxyhexahydrocannabinol-C5 [(−)-Cannabiripsol]
(CBR)
Cannabichromanone C-C5
(−)-6a,7,10a-Trihydroxy-49-tetrahydrocannabinol-C5 [(−)-Cannabitetrol] (CBTT)
Cannabichromanone B-C5
8,9-Dihydroxy-Δ6a(10a)-tetrahydrocannabinol-C5 (8,9-Di-OHCBT)
(±)-4-acetoxycannabichromene-C5
2-acetoxy-6-geranyl-3-n-pentyl-1,4-benzoquinone-C5
11-Acetoxy-Δ9-Tetrahydrocannabinol-C5 (11-OAc-Δ9-THC)
5-acetyl-4-hydroxycannabigerol-C5
4-acetoxy-2-geranyl-5-hydroxy-3-npentylphenol-C5
(−)-trans-10-Ethoxy-9-hydroxy-Δ6a(10a)-tetrahydrocannabinol-C5 ((−)-trans-CBTOet)
4-acetoxy-2-geranyl-5-hydroxy-3-npropylphenol-C5
sesquicannabigerol-C5 (SesquiCBG)
carmagerol-C5
4-terpenyl cannabinolate-C5
β-fenchyl-Δ9-tetrahydrocannabinolate-C5
α-fenchyl-Δ9-tetrahydrocannabinolate-C5
epi-bornyl-Δ9-tetrahydrocannabinolate-C5
bornyl-Δ9-tetrahydrocannabinolate-C5
α-terpeny1-Δ9-tetrahydrocannabinolate-C5
4-terpenyl-Δ9-tetrahydrocannabinolate-C5

Cannabichromene (CBC) (CAS NO. 20675-51-8) as used herein encompasses native CBC (i.e. originating from the Cannabis plant), or synthetic analogs or derivatives thereof. According to specific embodiments, any CBC analog may be used in accordance with specific embodiments of the present teachings as long as it comprises the anti-cancer activity described herein (alone, or as part of a fraction or composition discussed herein).

Non-limiting examples of CBC analogs include isocannabichromene, cannabichromene-c0, cannabichromene-cl, isocannabichromene-c0, CBCan. CBC or CBC-like derivatives found in Rhododendron anthopogonoides.

According to specific embodiments, the CBC comprises native CBC.

Pure or synthetic CBC can be commercially obtained from e.g. Restek catalog no. 34092, Cayman chemicals catalog no. 26252-10/5/50, Santa Cruz Biotechnology catalog no. sc-504602.

Tetrahydrocannabinol (THC) (CAS No. 1972-08-3) as used herein encompasses native THC (i.e. originating from the Cannabis plant), or synthetic analogs or derivatives thereof. According to specific embodiments, any THC analog may be used in accordance with the present teachings as long as it comprises the anti-cancer activity described herein (alone, or as part of the composition discussed herein).

According to specific embodiments, the THC comprises native THC.

Pure or synthetic THC can be commercially obtained from e.g. Restek catalog no. 34067.

The term THC does not include tetrahydrocannabinolic acid (THCA).

Cannabidiol (CBD) (CAS No. 13956-29-1), as used herein, encompasses native CBD (i.e. originating from the Cannabis plant), or synthetic or naturally occurring analogs or derivatives thereof. According to specific embodiments, any CBD analog may be used in accordance with specific embodiments of the present teachings as long as it comprises the anti-cancer activity described herein (alone, or as part of a composition discussed herein).

Exemplary CBD analogs include, but are not limited to, (−)-DMH-CBD-11-oic acid, HU-308 (commercially available e.g. from Tocris Bioscience, 3088), 0-1602 (commercially available e.g. from Tocris Bioscience 2797/10), DMH-CBD (commercially available e.g. from Tocris Bioscience, 1481) [as discussed in detail in Burstein S, Bioorg Med Chem. (2015) 23(7): 1377-85], Abn-CBD, HUF-101. CBDV, CBDM, CBND-C5, CBND-C3, 6-Hydroxy-CBD-triacetate or CBD-aldehyde-diacetate [as discussed in detail in An Overview on Medicinal Chemistry of Synthetic and Natural Derivatives of Cannabidiol, Frontiers in Pharmacology, June 2017|Volume 8|Article 422].

According to specific embodiments, the CBD is not CBDV.

According to specific embodiments, the CBD comprises native CBD.

Pure or synthetic CBD can be commercially obtained from e.g. Restek catalog no. 34011.

Tetrahydrocannabinolic acid (THCA) (CAS No: 23978-85-0), as used herein, refers to Δ9-tetrahydrocannabinolic acid, the precursor of tetrahydrocannabinol (THC). The term THCA as used herein encompasses native THCA (i.e. originating from the Cannabis plant), or synthetic analogs or derivatives thereof. According to specific embodiments, any THCA analog may be used in accordance with the present teachings as long as it comprises the anti-cancer activity described herein (alone, or as part of the composition discussed herein).

Exemplary THCA analogs include, but are not limited to, 11-OH-delta9-THCA-A and 11-Nor-delta9-THCA-A carboxylic acid [as discussed in detail in Guillermo Moreno-Sanz, Critical Review and Novel Therapeutic Perspectives of D9-Tetrahydrocannabinolic Acid A, Cannabis and Cannabinoid Research Volume 1.1, (2016)].

According to specific embodiments, the THCA comprises native THCA.

Pure or synthetic THCA can be commercially obtained from e.g. Restek catalog no. 34093.

The term THCA does not include tetrahydrocannabinol (THC).

Tetrahydrocannabivarin (THCV) (CAS No. 31262-37-0), as used herein, encompasses native THCV (i.e. originating from the Cannabis plant), or synthetic or naturally occurring analogs or derivatives thereof. According to specific embodiments, any THCV analog may be used in accordance with the present teachings as long as it comprises the anti-cancer activity described herein (alone, or as part of the composition discussed herein).

An exemplary THCV analog include, but is not limited to, 48-THCV.

According to specific embodiments, the THCV comprises native THCV.

Pure or synthetic THCV can be commercially obtained from e.g. Restek catalog no. 34100.

Cannabinol (CBN) (CAS NO. 521-35-7), as used herein, encompasses native CBN (i.e. originating from the Cannabis plant), or synthetic analogs or derivatives thereof. According to specific embodiments, any CBN analog may be used in accordance with the present teachings as long as it comprises the anti-cancer activity described herein (alone, or as part of the composition discussed herein).

According to specific embodiments, the CBN comprises native CBN.

Pure or synthetic CBN can be commercially obtained from e.g. Restek catalog no. 34010.

Cannabigerol (CBG) (CAS No. 25654-31-3), as used herein, encompasses native CBG (i.e. originating from the Cannabis plant), or synthetic or naturally occurring analogs or derivatives thereof. According to specific embodiments, any CBG analog may be used in accordance with the present teachings as long as it comprises the anti-cancer activity described herein (alone, or as part of the composition discussed herein).

According to specific embodiments, the CBG comprises native CBG.

Pure or synthetic CBG can be commercially obtained from e.g. Restek catalog no. 34091.

Cannabidiolic acid (CBDA) (CAS No. 1244-58-2), as used herein, encompasses native CBDA (i.e. originating from the Cannabis plant), or synthetic analogs or derivatives thereof. According to specific embodiments, any CBDA analog may be used in accordance with the present teachings as long as it comprises the anti-cancer activity described herein (alone, or as part of the composition discussed herein).

According to specific embodiments, the CBDA comprises native CBDA.

Pure or synthetic CBDA can be commercially obtained from e.g. Restek catalog no. 34099.

Cannabidivarin (CBDV) (CAS No. 24274-48-4), as used herein, encompasses native CBDV (i.e. originating from the Cannabis plant), or synthetic analogs or derivatives thereof. According to specific embodiments, any CBDV analog may be used in accordance with the present teachings as long as it comprises the anti-cancer activity described herein (alone, or as part of the composition discussed herein).

According to specific embodiments, the CBDV comprises native CBDV.

Cannabidivarinic Acid (CBDVA) (CAS No. 31932-13-5), as used herein, encompasses native CBDVA (i.e. originating from the Cannabis plant), or synthetic analogs or derivatives thereof. According to specific embodiments, any CBDVA analog may be used in accordance with the present teachings as long as it comprises the anti-cancer activity described herein (alone, or as part of the composition discussed herein).

According to specific embodiments, the CBDVA comprises native CBDVA.

Pure or synthetic CBDVA can be commercially obtained from e.g. Restek catalog no 34094.

The compositions disclosed herein comprise cannabinoids at percentages as described herein.

As used herein, a “percent (%) of a cannabinoid” in the fractions and compositions disclosed herein refers to the % calculated from concentration (w/v) of the recited cannabinoid out of the total cannabinoids, active ingredients or compounds in the fraction or composition, as can be determined by the peak area according to a HPLC profile of the fraction or composition.

According to specific embodiments, the % of a cannabinoid is out of the total cannabinoids i.e., phytocannabinoids (and only the cannabinoids i.e., phytocannabinoids) in the fraction or composition.

Methods of determining presence or absence of a compounds in the composition, as well as the concentration of a compound in the composition are well known in the art, such as, but not limited to ultraviolet-visible spectroscopy (“UV-Vis”), infrared spectroscopy (“IR”), and the like; mass-spectrometry (“MS”) methods such as, but not limited to, time-of-flight MS; quadrupole MS; electrospray MS, Fourier-transform MS, Matrix-Assisted Laser Desorption/Ionization (“MALDI”), and the like; chromatographic methods such as, but not limited to, gas-chromatography (“GC”), liquid chromatograph (“LC”), high-performance liquid chromatography (“HPLC”), and the like; and combinations thereof (e.g., GC/MS, LC/MS, HPLC/UV-Vis, and the like), and other analytical methods known to persons of ordinary skill in the art.

According to specific embodiments, determining presence or absence of a compound in the composition and/or the concentration of a compound in the composition is effected by analytical high pressure liquid chromatography (HPLC).

According to specific embodiments, composition (i) comprises in the cannabinoids at least 80% CBC and optionally tetrahydrocannabinol THC. It will be appreciated that percentage (%) relate to percentage of cannabinoids in the composition.

According to specific embodiments composition (i) comprises in the cannabinoids at least 80% CBC.

According to specific embodiments composition (i) comprises in the cannabinoids at least 80% CBC and THC (i.e. CBC and THC together constitute at least 80% of the composition).

According to specific embodiments, the at least 80% in composition (i) is at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%.

According to a specific embodiment, the at least 80% in composition (i) is at least 90%.

According to a specific embodiment, the at least 80% in composition (i) is at least 95%.

According to specific embodiments, composition (i) comprises both CBC and THC.

According to specific embodiments, when composition (i) comprises both CBC and THC then the composition comprises in the cannabinoids at least 5% CBC.

According to specific embodiments, composition (i) comprises in the cannabinoids at least 10% CBC.

According to specific embodiments, composition (i) comprises in the cannabinoids at least 20%, at least 30%, at least 40%, at least 50% CBC.

According to specific embodiments, when composition (i) comprises both CBC and THC, CBC is the most abundant (or predominant) cannabinoid in composition.

According to specific embodiments, composition (i) comprises in the cannabinoids at least 60% or at least 70% CBC.

According to specific embodiments, composition (i) comprises in the cannabinoids at least 80%, at least 81%, at least 82%, at least 83%, at least 84% or at least 85% CBC.

According to specific embodiments, composition (i) comprises in the cannabinoids 80-90% CBC.

According to specific embodiments, composition (i) comprises in the cannabinoids 80-85% CBC.

According to specific embodiments, composition (i) comprises in the cannabinoids about 85.8% CBC.

According to specific embodiments, composition (i) comprises in the cannabinoids 20-30% CBC.

According to specific embodiments, composition (i) comprises in the cannabinoids at least 5% THC.

According to specific embodiments, composition (i) comprises in the cannabinoids at least 6%, at least 7%, at least 8%, at least 9% THC.

According to specific embodiments, composition (i) comprises in the cannabinoids at least 10% THC.

According to specific embodiments, composition (i) comprises in the cannabinoids 10-20% THC.

According to specific embodiments, composition (i) comprises in the cannabinoids 12-18% or 13-16% THC.

According to specific embodiments, composition (i) comprises in the cannabinoids about 14.2% THC.

According to specific embodiments, composition (i) comprises in the cannabinoids at least 50%, at least 60%, at least 70% THC.

According to specific embodiments, composition (i) comprises in the cannabinoids 70-80% THC.

According to specific embodiments, CBC is the most abundant (or predominant) cannabinoid in composition (i).

According to specific embodiments, THC is the most abundant (or predominant) cannabinoid in composition (i)

According to specific embodiments, composition (i) comprises CBC and THC in a concentration ratio of 15:1-1:15.

According to specific embodiments, the concentration ratio is determined by g/1:g/1 or μg/ml:μg/ml.

According to specific embodiments, composition (i) comprises CBC and THC in a concentration ration of 15:1-1:10, 10:1-1:15, 10:1-1:10, 10:1-1:5; 5:1-1:10, 5:1-1:5, or 6:1-1:3.

According to specific embodiments, composition (i) comprises CBC and THC in a concentration ratio of 10:1-1:5.

According to specific embodiments, composition (i) comprises CBC and THC in a concentration ratio of 5:1-1:2.

According to specific embodiments, composition (i) comprises CBC and THC in a concentration ratio of about 6:1.

According to specific embodiments, composition (i) comprises CBC and THC in a concentration ratio of about 1:3.

According to specific embodiments, composition (i) comprises the cannabinoids listed in the GB10011-F7 composition of Table 1 in percentages as listed in the GB10011-F7 composition of Table 1±10%.

According to specific embodiments, composition (i) comprises the cannabinoids listed in the GB10011-F7 composition of Table 1 in percentages as listed in the GB10011-F7 composition of Table 1.

According to specific embodiments, composition (i) comprises in the cannabinoids 20-30% CBC and 70-80% THC.

According to specific embodiments, composition (i) is provided such that a concentration of CBC is at least 1 μg/ml, at least 3 μg/ml, at least 5 μg/ml, at least 7 μg/ml, at least 9 μg/ml, at least 11 μg/ml, each possibility represents a separate embodiment of the invention.

According to specific embodiments, composition (i) is provided such that a concentration of THC is at least 1 μg/ml, at least 2 μg/ml, at least 3 μg/ml, at least 5 μg/ml, at least 9 μg/ml, at least 11 μg/ml, at least 13 μg/ml, each possibility represents a separate embodiment of the invention.

According to specific embodiments, composition (i) is provided such that a concentration of CBC is at least 7 μg/ml and a concentration of THC is at least 2 μg/ml.

According to specific embodiments, composition (i) is provided such that a concentration of CBC is at least 1 μg/ml and a concentration of THC is at least 13 μg/ml.

According to specific embodiments, composition (i) is provided such that a concentration of CBC is about 10-15 μg/ml and a concentration of THC is about 1-5 μg/ml.

According to specific embodiments, composition (i) is provided such that a concentration of CBC is about 7-12 μg/ml and a concentration of THC is about 5-13 μg/ml.

According to specific embodiments, composition (i) comprises CBC and THC in a concentration and a concentration ratio according to Table 2±10%.

According to a specific embodiment, composition (i) comprises CBC and THC in a ratio of about 6:1 and comprises at least 11 μg/ml CBC and at least 2 μg/ml THC.

According to specific embodiments, composition (i) is devoid of CBD.

According to specific embodiments, composition (i) does not comprise more than 0.5%, more than 1%, more than 5%, more than 10% CBD.

According to specific embodiments, composition (i) is devoid of CBD, CBG and/or THCA.

According to specific embodiments, composition (i) is devoid of CBD, CBG, THCV and/or CBN.

According to specific embodiments, composition (i) is devoid of cannabinoids other than CBC and THC.

According to specific embodiments, composition (ii) comprises in the cannabinoids at least 50% CBD and/or THC (i.e. either CBD constitutes 50% in the cannabinoids of the composition, or THC constitutes at least 50% in the cannabinoids of the composition, or CBD and THC together constitute at least 50% in the cannabinoids of the composition), and at least one cannabinoid selected from the group consisting of CBDVA, CBG, THCV, CBN, CBC, CBDA, THCA and CBDV.

According to specific embodiments, composition (ii) comprises CBG.

According to specific embodiments, composition (ii) comprises CBN.

According to specific embodiments, composition (ii) comprises THCV.

According to specific embodiments, composition (ii) comprises CBC.

According to specific embodiments, the at least one cannabinoid in composition (ii) comprises at least two, at least three or at least four of the recited cannabinoids.

According to specific embodiments, composition (ii) comprises CBG and THCV.

According to specific embodiments, composition (ii) comprises CBG and CBN.

According to specific embodiments, composition (ii) further comprises CBGA.

According to specific embodiments, the at least 50% of the cannabinoids in composition (ii) is at least 55%, at least 60%, at least 65%, at least 70%, at least 75%, at least 80%, at least 85% or at least 90%.

According to specific embodiments, composition (ii) comprises THC and CBD.

According to specific embodiments, CBD is the most abundant cannabinoid in composition (ii).

According to specific embodiments, composition (ii) comprises in the cannabinoids at least 50%, at least 55%, at least 60%, at least 65%, at least 70%, at least 75%, at least 80%, at least 85%, at least 90% CBD.

According to other specific embodiments, THC is the most abundant cannabinoid in composition (ii).

According to specific embodiments, composition (ii) comprises in the cannabinoids at least 50%, at least 55%, at least 60%, at least 65%, at least 70%, at least 75%, at least 80%, at least 85%, at least 90% THC.

According to specific embodiments, composition (ii) comprises the cannabinoids listed in the crude extract composition of Table 1.

According to specific embodiments, composition (ii) is devoid of cannabinoids other than the cannabinoids listed in the crude extract composition of Table 1.

According to specific embodiments, composition (ii) comprises in the cannabinoids at least 2%, at least 3% or at least 4% CBG.

According to specific embodiments, composition (ii) comprises in the cannabinoids less than 10%, less than 8%, less than 6% or less than 5% CBG.

According to specific embodiments, composition (ii) comprises in the cannabinoids 4-6% CBG.

According to specific embodiments, composition (ii) comprises in the cannabinoids at least 4% CBDVA.

According to specific embodiments, composition (ii) comprises in the cannabinoids less than 10%, less than 8%, less than 5% or less than 5% CBDVA.

According to specific embodiments, composition (ii) comprises in the cannabinoids 4-6% CBDVA.

According to specific embodiments, composition (ii) comprises in the cannabinoids at least 2% CBC.

According to specific embodiments, composition (ii) comprises in the cannabinoids less than 10%, less than 8%, less than 5%, less than 5% or less than 4% CBC.

According to specific embodiments, composition (ii) comprises in the cannabinoids 2-4% CBC.

According to specific embodiments, composition (ii) comprises in the cannabinoids at least 40% or at least 45% CBD.

According to specific embodiments, composition (ii) comprises in the cannabinoids less than 60% or less than 55% CBD.

According to specific embodiments, composition (ii) comprises in the cannabinoids 45-55% CBD.

According to specific embodiments, composition (ii) comprises in the cannabinoids at least 20% THC.

According to specific embodiments, composition (ii) comprises in the cannabinoids less than 40% or less than 30% THC.

According to specific embodiments, composition (ii) comprises in the cannabinoids 20-30% a THC.

According to specific embodiments, composition (ii) comprises in the cannabinoids less than 1%, less than 0.5% or less than 0.2% THCV.

According to specific embodiments, composition (ii) comprises in the cannabinoids 0.005-0.2% THCV.

According to specific embodiments, composition (ii) comprises less than 1% or less than 0.6% CBN.

According to specific embodiments, composition (ii) comprises in the cannabinoids 0.1-1% CBN.

According to specific embodiments, composition (ii) comprises percentages of cannabinoids as listed in the crude extract composition of Table 1±10%.

According to specific embodiments, composition (ii) comprises percentages of cannabinoids as listed in the crude extract composition of Table 1.

According to specific embodiments, composition (ii) comprises the cannabinoids listed in the GB10011-F4 composition of Table 1.

According to specific embodiments, composition (ii) is devoid of cannabinoids other than the cannabinoids listed in the GB10011-F4 composition of Table 1.

According to specific embodiments, composition (ii) comprises in the cannabinoids at least 90% CBD.

According to specific embodiments, composition (ii) comprises in the cannabinoids 90-98% CBD.

According to specific embodiments, composition (ii) comprises in the cannabinoids at least 2%, at least 3% or at least 4% CBG.

According to specific embodiments, composition (ii) comprises in the cannabinoids less than 10%, less than 6% or less than 5% CBG.

According to specific embodiments, composition (ii) comprises in the cannabinoids 4-6% or 3-5% CBG.

According to specific embodiments, composition (ii) comprises in the cannabinoids less than 1%, less than 0.5% or less than 0.2% THCV.

According to specific embodiments, composition (ii) comprises in the cannabinoids 0.05-0.2% THCV.

According to specific embodiments, composition (ii) comprises percentages of cannabinoids as listed in the GB10011-F4 composition of Table 1±10%.

According to specific embodiments, composition (ii) comprises percentages of cannabinoids as listed in the GB10011-F4 composition of Table 1.

According to specific embodiments, composition (ii) comprises the cannabinoids listed in the GB10011-F5 composition of Table 1.

According to specific embodiments, composition (ii) is devoid of cannabinoids other than the cannabinoids listed in the GB10011-F5 composition of Table 1.

According to specific embodiments, composition (ii) comprises in the cannabinoids less than 20% or less than 15% CBD.

According to specific embodiments, composition (ii) comprises in the cannabinoids 10-15% CBD.

According to specific embodiments, composition (ii) comprises in the cannabinoids at least 1% or at least 2% CBDA.

According to specific embodiments, composition (ii) comprises in the cannabinoids less than 10%, less than 5% or less than 3% CBDA.

According to specific embodiments, composition (ii) comprises in the cannabinoids 2-3% CBDA.

According to specific embodiments, composition (ii) comprises in the cannabinoids less than 60% or less than 55% THC.

According to specific embodiments, composition (ii) comprises in the cannabinoids 45-55% THC.

According to specific embodiments, composition (ii) comprises in the cannabinoids thereof at least 5% THCA.

According to specific embodiments, composition (ii) comprises in the cannabinoids less than 15% or less than 10% THCA.

According to specific embodiments, composition (ii) comprises in the cannabinoids 5-10% THCA.

According to specific embodiments, composition (ii) comprises in the cannabinoids less than 2% or less than 1% CBDV.

According to specific embodiments, composition (ii) comprises in the cannabinoids 0.5-1% CBDV.

According to specific embodiments, composition (ii) comprises in the cannabinoids at least 1% or at least 2% THCV.

According to specific embodiments, composition (ii) comprises in the cannabinoids less than 10% or less than 5% THCV.

According to specific embodiments, composition (ii) comprises in the cannabinoids 2-3% THCV.

According to specific embodiments, composition (ii) comprises in the cannabinoids at least 1%, at least 2% or at least 3% CBG.

According to specific embodiments, composition (ii) comprises in the cannabinoids less than 10% or less than 5% CBG.

According to specific embodiments, composition (ii) comprises in the cannabinoids 3-4% CBG.

According to specific embodiments, composition (ii) comprises in the cannabinoids at least 10% or at least 15% CBN.

According to specific embodiments, composition (ii) comprises in the cannabinoids less than 30%, less than 25% or less than 20% CBN.

According to specific embodiments, composition (ii) comprises in the cannabinoids 15-20% CBN.

According to specific embodiments, composition (ii) comprises percentages of cannabinoids as listed in the GB10011-F5 composition of Table 1±10%.

According to specific embodiments, composition (ii) comprises percentages of cannabinoids as listed in the GB10011-F5 composition of Table 1.

According to specific embodiments, composition (ii) comprises the cannabinoids listed in the GB10011-F6 composition of Table 1.

According to specific embodiments, composition (ii) is devoid of cannabinoids other than the cannabinoids listed in the GB10011-F6 composition of Table 1.

According to specific embodiments, composition (ii) comprises in the cannabinoids at least 90%, at least 95%, at least 96%, at least 97% or at least 98% THC.

According to specific embodiments, composition (ii) comprises in the cannabinoids 97-99% a THC.

According to specific embodiments, composition (ii) comprises in the cannabinoids less than 1% or less than 0.5% CBG.

According to specific embodiments, composition (ii) comprises in the cannabinoids 0.3-0.4% CBG.

According to specific embodiments, composition (ii) comprises in the cannabinoids less than 1% or less than 1% CBD.

According to specific embodiments, composition (ii) comprises in the cannabinoids 0.1-0.3% CBD.

According to specific embodiments, composition (ii) comprises in the cannabinoids less than 2% or less than 1% CBN.

According to specific embodiments, composition (ii) comprises in the cannabinoids 0.5-0.8% CBN.

According to specific embodiments, composition (ii) comprises % of cannabinoids as listed in the GB10011-F6 composition of Table 1±10%.

According to specific embodiments, composition (ii) comprises % of cannabinoids as listed in the GB10011-F6 composition of Table 1.

According to specific embodiments, composition (ii) comprises the cannabinoids listed in the sCBD crude extract composition of Table 6.

According to specific embodiments, composition (ii) is devoid of cannabinoids other than the cannabinoids listed in the sCBD crude extract composition of Table 6.

According to specific embodiments, composition (ii) comprises percentages of cannabinoids as listed in the sCBD crude extract composition of Table 6±10%.

According to specific embodiments, composition (ii) comprises percentages of cannabinoids as listed in the sCBD crude extract composition of Table 6.

According to specific embodiments, composition (ii) comprises the cannabinoids listed in the PARIS crude extract composition of Table 6.

According to specific embodiments, composition (ii) is devoid of cannabinoids other than the cannabinoids listed in the PARIS crude extract composition of Table 6.

According to specific embodiments, composition (ii) comprises percentages of cannabinoids as listed in the PARIS crude extract composition of Table 6±10%.

According to specific embodiments, composition (ii) comprises percentages of cannabinoids as listed in the PARIS crude extract composition of Table 6.

According to specific embodiments, composition (ii) comprises the cannabinoids listed in the DQ crude extract composition of Table 6.

According to specific embodiments, composition (ii) is devoid of cannabinoids other than the cannabinoids listed in the DQ crude extract composition of Table 6.

According to specific embodiments, composition (ii) comprises percentages of cannabinoids as listed in the DQ crude extract composition of Table 6±10%.

According to specific embodiments, composition (ii) comprises percentages of cannabinoids as listed in the DQ crude extract composition of Table 6.

According to specific embodiments, composition (ii) comprises the cannabinoids listed in the DQ F7 composition of Table 6.

According to specific embodiments, composition (ii) is devoid of cannabinoids other than the cannabinoids listed in the DQ F7 composition of Table 6.

According to specific embodiments, composition (ii) comprises percentages of cannabinoids as listed in the DQ F7 composition of Table 6±10%.

According to specific embodiments, composition (ii) comprises percentages of cannabinoids as listed in the DQ F7 composition of Table 6. According to specific embodiments, the composition disclosed herein is devoid of active compounds other than cannabinoids.

According to other specific embodiments, the composition comprises active compounds other than cannabinoids.

Thus, according to specific embodiments, less than 50%, less than 40%, less than 30%, less than 20% or less than 10% of the active compounds in the composition are cannabinoids.

According to a specific embodiment, 5-10% of the active compounds in the composition are cannabinoids.

According to specific embodiments, at least 50% of the active compounds in the composition are cannabinoids.

According to specific embodiments, at least 55%, at least 60% or at least 65%, at least 70%, at least 75% of the active compounds in the composition are cannabinoids.

According to specific embodiments, 50-100%, 60-90% or 60-80% of the active compounds in the composition are cannabinoids.

According to specific embodiments, 90-100% of the active compounds in the composition are cannabinoids.

The composition of some embodiments of the invention may be a synthetic composition, a compositions comprising purified cannabinoids or a fraction of a cannabis extract.

According to specific embodiments, the composition a cannabis extract (e.g. crude extract not subjected to fractionation).

According to specific embodiments, the composition is not a cannabis extract.

According to specific embodiments, the composition contains 2-4 cannabinoids.

According to specific embodiments, the composition is characterized by increased stability, increased bioavailability, less side effects and/or better pharmacokinetic properties as compared to a cannabis extract.

According to specific embodiments, the composition is a synthetic composition.

Herein, the term “synthetic composition” refers to a chemically defined composition which can include active ingredients which are chemically synthesized and/or purified to a level of purity of at least 99%.

As used herein “a chemically defined composition” refers to a composition in which all the constituents are known by structure and optionally concentration.

According to specific embodiments, the cannabinoids are purified from cannabis.

According to specific embodiments, the cannabinoids are synthetic cannabinoids.

According to specific embodiments, the composition is a cannabis derived fraction.

As used herein “a fraction” refers to a portion of the extract that contains only certain chemical ingredients of the extract but not all.

According to specific embodiments, the composition is a liquid chromatography fraction of a cannabis extract.

According to specific embodiments, the liquid chromatography comprises high pressure liquid chromatography (HPLC) or flash chromatography.

According to specific embodiments, the liquid chromatography fraction of cannabis extract comprises a liquid chromatography pooled fractions of cannabis extract comprising active ingredients detectable by a detector operated at 220 nm, wherein the active ingredients comprise the compounds disclosed herein.

According to specific embodiments, the liquid chromatography fraction is obtainable by subjecting the cannabis extract to flash chromatography comprising a Flash chromatography apparatus equipped with a diode array detector, a C18 functionalized silica column, a 55% to 100% water and methanol gradient at a flow rate of 30 ml/min.

According to specific embodiments, the fraction of composition (i) is collected between about 22-22.5 minutes of the flash chromatography.

According to specific embodiments, the fraction of composition (ii) is collected between about 15-17 minutes of the flash chromatography.

According to specific embodiments, the fraction of composition (ii) is collected between about 17-20 minutes of the flash chromatography.

According to specific embodiments, the fraction of composition (ii) is collected between about 20-22 minutes of the flash chromatography.

According to specific embodiments, the detector is a diode array detector.

According to specific embodiments, the liquid chromatography fraction is obtainable by subjecting the cannabis extract to flash chromatography comprising a Flash chromatography apparatus equipped with a diode array detector, a C18, functionalized silica column, a 55% to 100% water and methanol gradient at a flow rate of 60 ml/min.

According to specific embodiments, the fraction of composition (ii) is collected between about 12-14 minutes of the flash chromatography.

According to specific embodiments, the detector is a 1260 MWD-VL detector.

A non-limiting example of liquid chromatography fractionation that can be effect with specific embodiments of the invention is described in the Examples section which follows, which serves as an integral part of the specification.

Cannabis is a genus of flowering plants in the family Cannabaceae that includes three different species, Cannabis sativa, Cannabis indica and Cannabis ruderalis. The term Cannabis encompasses wild type Cannabis and also variants thereof, including cannabis chemovars which naturally contain different amounts of the individual cannabinoids. For example, some Cannabis strains have been selectively bred to produce high or low levels of THC or CBD and other cannabinoids.

According to specific embodiments, the Cannabis plant is a wild-type plant.

According to specific embodiments, the Cannabis plant is transgenic.

According to specific embodiments, the Cannabis plant is genomically edited.

According to specific embodiments, the Cannabis plant is Cannabis sativa (C. sativa).

According to specific embodiments, the Cannabis plant is C. sativa strain IGB chemovar 10011 (obtained from the Israel Gene Bank (IGB) collection, Israel).

According to specific embodiments, the Cannabis plant is C. sativa strain is super CBD (sCBD, IMC, Israel).

According to specific embodiments, the Cannabis plant is C. sativa strain is, Dairy Queen (DQ, IMC, Israel).

According to specific embodiments, the Cannabis plant is C. sativa strain is PARIS (IMC, Israel).

The extract may be derived from a cultivated Cannabis plant (i.e. not grown in their natural habitat) or may be derived from Cannabis plants which grow in the wild.

The tissue of the Cannabis plant from which the extract is typically obtained is the inflorescence. Accordingly, the extract may be obtained from the complete flower head of a plant including stems, stalks, bracts, and flowers. However, it will be appreciated that a cannabis extract of some embodiments the invention may be obtained from only part of the inflorescence, such as from the bracts and/or flowers.

A non-limiting example of growing the plant and obtaining the extract, which can be used with specific embodiments of the invention is described in the Examples section which follows, which serve as an integral part of the specification.

According to specific embodiments, the extract is obtained from a fresh plant (i.e. a plant not heated prior to the extraction process). Fresh plants include plants taken immediately following harvesting (e.g., up to an hour or several hours) for extraction as well as plants frozen immediately after harvesting (e.g. at about −70° C. to −90° C., e.g. at −80° C., for any required length of time) prior to extraction.

According to specific embodiments, the extract is obtained from fresh inflorescence.

According to specific embodiments, the extract is obtained from a frozen inflorescence (e.g. frozen immediately after harvesting at about −70° C. to −90° C., e.g. at −80° C., for any required length of time). Thus, for example, the extract may be obtained from a cryopreserved inflorescence, or from an inflorescence frozen in liquid nitrogen or in dry ice.

According to specific embodiments, the extract is obtained from an inflorescence which has not been subjected to heating (such as heating at e.g. at 120° C. to 180° C., e.g. at 150° C., for any length of time, such as for 1-5 hours).

According to specific embodiments, the extract is obtained from dry Cannabis inflorescence. Drying the inflorescence may be carried out using any method known in the art, such as by pulverizing with liquid nitrogen or with dry-ice/alcohol mixture.

According to specific embodiments, the dry inflorescence is obtained from the grower.

According to specific embodiments, the polar solvent comprises a polar, protic solvent (e.g., ethanol or methanol). In some embodiments, the polar solvent comprises a polar, aprotic solvent (e.g., acetone). Polar solvents suitable for use with specific embodiments of the present invention include, but are not limited to, ethanol, methanol, n-propanol, iso-propanol, a butanol, a pentanol, acetone, methylethylketone, ethylacetate, acetonitrile, tetrahydrofuran, dimethylformamide, dimethylsulfoxide, water, and combinations thereof.

According to specific embodiments, the polar solvent is ethanol (e.g. absolute ethanol i.e. above 99.8%, or in the range of 99-70% in water).

The concentration or amount of a polar solvent used to Cannabis inflorescence can be varied. Generally, the ratio of a Cannabis inflorescence to a polar solvent (weight to volume) is the amount of a polar solvent sufficient to extract about 70% or more, about 75% or more, about 85% or more, about 90% or more, about 95% or more, about 97% or more, or about 99% or more of a composition having a cytotoxic activity. In some embodiments, the ratio of polar solvent to Cannabis inflorescence is about 1:2 to about 1:20 (w/v), e.g. about 1:4 to about 1:10 (w/v).

In particular embodiments, the extract is an ethanol extract.

In particular embodiments, absolute ethanol is added to the inflorescence at a sample-to-absolute ethanol ratio of 1:4 (w/v).

In some embodiments, the Cannabis inflorescence is contacted with a polar solvent (e.g. ethanol) for about 15 minutes or more, about 30 minutes or more, about 1 hour or more, about 2 hours or more, or about 5 hours or more.

According to specific embodiments, the Cannabis inflorescence is contacted with a polar solvent (e.g. ethanol) for about 30 minutes.

Temperature can also be controlled during the contacting. In some embodiments, the Cannabis inflorescence is contacted with a polar solvent at temperature of about 15° C. to about 35° C., or about 20° C. to about 25° C.

According to specific embodiments, the Cannabis inflorescence is contacted with a polar solvent (e.g. ethanol) while being constantly mixed e.g. on a shaker.

In some embodiments, the process of obtaining the composition of some embodiments of the present invention comprises isolating a liquid extract (i.e. filtered extract) from the mixture (i.e. crude extract) comprising the liquid extract and solids. Suitable means for isolating the liquid extract (i.e. filtered extract) include those known in the art of organic synthesis and include, but are not limited to, gravity filtration, suction and/or vacuum filtration, centrifuging, setting and decanting, and the like. In some embodiments, the isolating comprises filtering a liquid extract through a porous membrane, syringe, sponge, zeolite, paper, or the like having a pore size of about 1-5 μm, about 0.5-5 μm, about 0.1-5 μm, about 1-2 μm, about 0.5-2 μm, about 0.1-2 μm, about 0.5-1 μm, about 0.1-1 μm, about 0.25-0.45 μm, or about 0.1-0.5 μm (e.g. about 2 μm, about 1 μm, about 0.45 μm, or about 0.25 μm).

According to a specific embodiment, the crude extract is filtered through a 0.45-μm syringe filter such as that commercially available from Merck, Darmstadt, Germany.

According specific embodiments, process of obtaining the composition of some embodiments of the present invention comprises drying (i.e. removal of the polar solvent) and/or freezing the filtered extract following generation thereof.

The method for drying the filtered extract (i.e. removing the polar solvent) is not particularly limited, and can include solvent evaporation at a reduced pressure (e.g., sub-atmospheric pressure) and/or an elevated temperature (e.g., above about 25° C.). In some embodiments, it can be difficult to completely remove a polar solvent from a liquid extract by standard solvent removal procedures such as evaporation. In some embodiments, processes such as co-evaporation, lyophilization, and the like can be used to completely remove the polar solvent from a liquid fraction to form a dry powder, dry pellet, dry granulate, paste, and the like. According to a specific embodiment, the polar solvent is evaporated with a vacuum evaporator.

According to specific embodiments, the extract (e.g. the filtered extract) is subjected to a decarboxylation step. Decarboxylation may be effected by heating the extract in a pressure tube in the oven at 220° C. for 10 minutes.

Following generation of the filtered extract, specific embodiments of the process of obtaining the composition of some embodiments of the present invention comprises additional purification steps so as to further purify active agents from the extract.

Thus, for example, fractionating the filtered extract. Fractionating can be performed by processes such as, but not limited to: column chromatography, preparative high performance liquid chromatography (“HPLC”), flash chromatography, reduced pressure distillation, and combinations thereof.

According to a specific embodiment, fractionating is performed by HPLC or flash chromatography.

In some embodiments, fractionating comprises re-suspending the filtered extract in a polar solvent (such as methanol, as discussed above), applying the polar extract to a separation column, and isolating the Cannabis fraction by column chromatography (e.g. preparative HPLC, flow cytometry).

An eluting solvent is applied to the separation column with the polar extract to elute fractions from the polar extract. Suitable eluting solvents for use include, but are not limited to, methanol, ethanol, propanol, acetone, acetic acid, carbon dioxide, methylethyl ketone, acetonitrile, butyronitrile, carbon dioxide, ethyl acetate, tetrahydrofuran, di-iso-propylether, ammonia, triethylamine, N,N-dimethylformamide, N,N-dimethylacetamide, and the like, and combinations thereof.

According to an alternative or an additional embodiment, liquid chromatography is performed on a reverse stationary phase.

According to an alternative or an additional embodiment, liquid chromatography comprises high performance liquid chromatography (HPLC) or flash chromatography, as further described hereinabove.

The fractions or extract obtained may be immediately used or stored until further use.

According to specific embodiments, the fraction or extract is kept frozen, e.g. in a freezer, until further use (e.g. at about −20° C. to −90° C., at about −70° C. to −90° C., e.g. at −80° C.), for any required length of time.

According to other specific embodiments, the fraction or extract is immediately used (e.g. within a few minutes e.g., up to 30 minutes).

The extracts and/or fractions may be used separately. Alternatively, different extracts (e.g. from different plants or from separate extraction procedures) may be pooled together. Likewise, different fractions (from the same extract, from different extracts, from different plants and/or from separate extraction procedures) may be pooled together.

The term “pooled” as used herein refers to collected from the liquid chromatography (e.g. HPLC, flash chromatography) either as a single fraction or a plurality of fractions.

The compositions of some embodiments of the invention have an anti-cancer effect on cancer cells e.g. urothelial carcinoma cells.

According to specific embodiments, the composition has a combined additive or synergistic anti-cancer effect on cancer cells e.g. urothelial carcinoma cells as compared to each of the recited cannabinoids (e.g. CBC, THC, CBD) when administered as a single agent.

According to specific embodiments, composition (i) has a combined synergistic cytotoxic activity on cancer cells e.g. urothelial carcinoma cells as compared to each of CBC and THC when administered as a single agent.

According to specific embodiments, the composition has a combined additive or synergistic anti-cancer effect on cancer cells e.g. urothelial carcinoma cells when administered with Mitomycin C (MMC) as compared to each of the composition and MMC when administered as a single agent.

As used herein the term “anti-cancer” refers to a statistically significant decrease in cancer growth and/or invasiveness in the presence of the composition in comparison to same in the absence of the composition. Such an effect may be manifested by, for example, but not limited to, reduced viability of cancer cells (e.g. urothelial carcinoma cells), induction of cancer cell cycle arrest, reduced migration of cancer cells (e.g. urothelial carcinoma cells), inhibition of sphere formation of cancer cells (e.g. urothelial carcinoma cells), inhibition of epithelial to mesenchymal transition of cancer cells (e.g. urothelial carcinoma cells). Alternatively or additionally, the anti-cancer effect may be manifested by improvement of one or more of the various physiological symptoms associated with cancer in a subject in need e.g. increased survival rate, increased progression without disease, decreased tumor size, decreased metastasis and the like.

According to a specific embodiment, the decrease is in at least 2%, 5%, 10%, 30%, 40% or even higher say, 50%, 60%, 70%, 80%, 90% or 100% as compared to same in the absence of the composition.

According to specific embodiments, the decrease is at least 1.5 fold, at least 2 fold, at least 3 fold, at least 5 fold, at least 10 fold, or at least 20 fold as compared to same in the absence of the composition.

Methods of determining an anti-cancer effect are well known in the art and are also described hereinbelow and in the Examples section which follows.

Non-limiting examples of methods of determining cell viability or cytotoxicity include the XTT assay, Annexin V assay [MEBCYTO Apoptosis Kit or ApoAlert® Annexin V Apoptosis Kit (Clontech Laboratories, Inc., CA, USA)]; the Senescence associated-β-galactosidase assay (Dimri GP, Lee X, et al. 1995. Proc Natl Acad Sci USA 92:9363-9367); MTT test which is based on the selective ability of living cells to reduce the yellow salt MTT (3-(4,5-dimethylthiazolyl-2)-2, 5-diphenyltetrazolium bromide) (Sigma, Aldrich St Louis, MO, USA) to a purple-blue insoluble formazan precipitate; the BrDu assay [Cell Proliferation ELISA BrdU colorimetric kit (Roche, Mannheim, Germany]; the TUNEL assay [Roche, Mannheim, Germany]; the as well as various RNA and protein detection methods (which detect level of expression and/or activity).

According to specific embodiments, the composition is capable of inducing apoptosis of cancer cells e.g. urothelial carcinoma cells.

According to specific embodiments, the composition is capable of inducing necrosis of cancer cells e.g. urothelial carcinoma cells.

A non-limiting example of a method of determining cell cycle arrest includes flow cytometry following PI staining.

Non-limiting examples of determining migration include the scratch assay, the transwell assay, cytoskeleton staining.

According to specific embodiments, the composition is capable of inducing disintegration of F-actin filaments in cancer cells e.g. urothelial carcinoma cells.

According to specific embodiments, the composition is capable of inhibiting cancer cells e.g. urothelial carcinoma cells sphere formation.

Methods of determining sphere formation are well known in the art and are further described in the Examples section which follows.

According to specific embodiments, the composition is capable of inhibiting epithelial to mesenchymal transition of cancer cells e.g. urothelial carcinoma cells.

Methods of determining sphere formation are well known in the art and are further described in the Examples section which follows, and include expression levels of E-cadherin and N-cadherin.

Consequently, according to an aspect of the present invention, there is provided a method of reducing viability, inducing cell cycle arrest and/or reducing migration of a cancerous cell, the method comprising contacting the cancerous cell with the composition disclosed herein.

According to specific embodiments, the contacting is effected in-vitro or ex-vivo.

According to other specific embodiments, the contacting is effected in-vivo.

According to specific embodiments, the method comprises determining the anti-cancer effect.

As the compositions disclosed herein are endowed with anti-cancer effects, specific embodiments suggest their use in treating cancer in a subject in need.

As used herein, the term “subject” or “subject in need thereof” includes mammals, preferably human beings at any age or gender which suffer from the pathology e.g. cancer e.g. urothelial cancer. According to specific embodiments, this term encompasses individuals who are at risk to develop the pathology.

According to specific embodiments, the subject is diagnosed with the pathology.

According to specific embodiments, the subject exhibit at least one symptom of the disease.

As used herein the term “treating” refers to curing, reversing, attenuating, alleviating, minimizing, suppressing or halting the deleterious effects of a disease or disorder (e.g. cancer e.g. urothelial cancer). Those of skill in the art will understand that various methodologies and assays can be used to assess the development of a pathology, and similarly, various methodologies and assays may be used to assess the reduction, remission or regression of a pathology (e.g. a malignancy).

According to a specific embodiment, treating is preventing.

As used herein, the term “preventing” refers to keeping a disease, disorder or condition from occurring in a subject who may be at risk for the disease, but has not yet been diagnosed as having the disease.

Non-limiting examples of cancers encompassed by specific embodiments of the invention can be any solid or non-solid cancer and/or cancer metastasis, including, but is not limiting to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

Precancers are well characterized and known in the art (refer, for example, to Berman J J. and Henson D E., 2003. Classifying the precancers: a metadata approach. BMC Med Inform Decis Mak. 3:8). Classes of precancers amenable to treatment via the method of the invention include acquired small or microscopic precancers, acquired large lesions with nuclear atypia, precursor lesions occurring with inherited hyperplastic syndromes that progress to cancer, and acquired diffuse hyperplasias and diffuse metaplasias. Examples of small or microscopic precancers include HGSIL (High grade squamous intraepithelial lesion of uterine cervix), AIN (anal intraepithelial neoplasia), dysplasia of vocal cord, aberrant crypts (of colon), PIN (prostatic intraepithelial neoplasia). Examples of acquired large lesions with nuclear atypia include tubular adenoma, AILD (angioimmunoblastic lymphadenopathy with dysproteinemia), atypical meningioma, gastric polyp, large plaque parapsoriasis, myelodysplasia, papillary transitional cell carcinoma in-situ, refractory anemia with excess blasts, and Schneiderian papilloma. Examples of precursor lesions occurring with inherited hyperplastic syndromes that progress to cancer include atypical mole syndrome, C cell adenomatosis and MEA. Examples of acquired diffuse hyperplasias and diffuse metaplasias include AIDS, atypical lymphoid hyperplasia, Paget's disease of bone, post-transplant lymphoproliferative disease and ulcerative colitis.

According to specific embodiments, the cancer is selected from the group consisting of urothelial cancer, uterine cervix carcinoma, prostate cancer and melanoma.

According to specific embodiments, the cancer is an urothelial cancer.

As used herein, the term “urothelial cancer” refers to a cancer originating from urothelial cells, which are transitional epithelium cells lining the surface of urogenital organs. Non-limiting examples of urothelial cancers include bladder, kidney, ureter, urethra and renal pelvis cancers.

According to a specific embodiment, the urothelial cancer is a bladder cancer. Each of the compositions or fractions described herein can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the cannabinoids and/or anti-cancer agents accountable for the biological effect.

According to specific embodiments, the cannabinoids are the only active ingredients in the composition.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as an oil-based formulation, tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

The pharmaceutical composition can be formulated for inhalation. For example, the compositions can be formulated as vapors or aerosols that can be inhaled into the lungs. Vapor formulations include liquid formulations that are vaporized when loaded into a suitable vaporization device.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For transdermal administration, the composition can be formulated in a form of a gel, a cream, an ointment, a paste, a lotion, a milk, a suspension, an aerosol, a spray, a foam, a serum, a swab, a pledget, a pad or a patch. Formulations for transdermal delivery can typically include carriers such as water, liquid alcohols, liquid glycols, liquid polyalkylene glycols, liquid esters, liquid amides, liquid protein hydrolysates, liquid alkylated protein hydrolysates, liquid lanolin, lanolin derivatives, glycerin, mineral oil, silicone, petroleum jelly, lanolin, fatty acids, vegetable oils, parabens, waxes, and like materials commonly employed in topical compositions. Various additives, known to those skilled in the art, may be included in the transdermal formulations of the invention. For example, solvents may be used to solubilize certain active ingredients substances. Other optional additives include skin permeation enhancers, opacifiers, anti-oxidants, gelling agents, thickening agents, stabilizers, and the like.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continues infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran.

Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

The compositions described herein can be administered to a subject in need thereof in a variety of other forms including a nutraceutical composition.

As used herein, a “nutraceutical composition” refers to any substance that may be considered a food or part of a food and provides medical or health benefits, including the prevention and treatment of disease. In some embodiments, a nutraceutical composition is intended to supplement the diet and contains at least one or more of the following ingredients: a vitamin; a mineral; an herb; a botanical; a fruit; a vegetable; an amino acid; or a concentrate, metabolite, constituent, or extract of any of the previously mentioned ingredients; and combinations thereof.

In some embodiments, a nutraceutical composition of the present invention can be administered as a “dietary supplement,” as defined by the U.S. Food and Drug Administration, which is a product taken by mouth that contains a “dietary ingredient” such as, but not limited to, a vitamin, a mineral, an herb or other botanical, an amino acid, and substances such as an enzyme, an organ tissue, a glandular, a metabolite, or an extract or concentrate thereof.

Non-limiting forms of nutraceutical compositions of the present invention include: a tablet, a capsule, a pill, a softgel, a gelcap, a liquid, a powder, a solution, a tincture, a suspension, a syrup, or other forms known to persons of skill in the art. A nutraceutical composition can also be in the form of a food, such as, but not limited to, a food bar, a beverage, a food gel, a food additive/supplement, a powder, a syrup, and combinations thereof.

Pharmaceutical or nutraceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g. cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals.

A non-limiting example of a model for urothelial cancer is the orthotopic mouse model, such as described e.g. in Wolfgang Jager et al. Methods Mol Biol (2018) 1655:177-197.

The doses determined in the mouse animal model can be converted for the treatment other species such as human and other animals diagnosed with the disease, using conversion Tables known to the skilled in the art.

The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

According to specific embodiments, the compositions disclosed herein can be administered to a subject in combination with other established or experimental therapeutic regimen to treat cancer including, but not limited to analgesics, chemotherapeutic agents, radiotherapeutic agents, cytotoxic therapies (conditioning), hormonal therapy and other treatment regimens (e.g., surgery) which are well known in the art.

Thus according to specific embodiments, the uses and methods described herein further comprise administering or contacting with an anti-cancer agent.

According to an additional or an alternative aspect of the present invention, there is provided an article of manufacture comprising the composition disclosed herein and an anti-cancer agent.

According to specific embodiments, the composition and said anti-cancer agent are in a co-formulation.

According to specific embodiments, the composition and said anti-cancer agent are in separate formulations.

Anti-cancer agents which can be used with specific embodiments of the invention are well known to the skilled in the art.

According to specific embodiments, the combination of the composition and the anti-cancer agent has a combined additive or synergistic anti-cancer effect on e.g. urothelial cells as compared to each of the composition and anti-cancer agent when administered as a single agent.

According to specific embodiments, the ratio between the composition and the anti-cancer agent is 10:1-1:10, 5:1-1:5 or 4:1-1:4.

According to specific embodiments, the concentration ratio between the composition and the anti-cancer agent is 4:1-1:4.

According to specific embodiments, the anti-cancer agent is a chemotherapy.

According to specific embodiments, the anti-cancer agent is an anti-urothelial carcinoma agent.

Non-limiting examples of anti-cancer agents that can be used with specific embodiments of the invention include Mitomycin C, Cisplatinum, carboplatinum, MVAC (methotrexate, vinblastine, Adriamycin, cisplatin), gemzar and Cisplatin-Gemzar.

According to a specific embodiment, the anti-cancer agent is Mitomycin C.

According to a specific embodiment, the anti-cancer agent is platinum based chemotherapy such as, but not limited to cisplatinum, carboplatinum.

According to a specific embodiment, the anti-cancer agent is Gemcitabin.

According to a specific embodiment, the anti-cancer agent is combined chemo protocol—MVAC (methotrexate, vinblastine, adriamycin, cisplatin).

According to a specific embodiment, the anti-cancer agent is combined chemo protocol—Cisplatin and gemcitabine.

According to specific embodiments, the anti-cancer agent is an immune-modulatory agent.

Immune-modulatory agents are well known in the art and include, but not limited to, chemokine receptor modulators, immune-check point modulators and cytokines.

Examples of check-point proteins that can be targeted according to specific embodiments of the invention include, but not limited to, PD1, PDL-1, B7H2, B7H3, B7H4, BTLA-4, HVEM, CTLA-4, CD80, CD86, LAG-3, TIM-3, KIR, IDO, CD19, OX40, OX40L, 4-1BB (CD137), 4-1BBL, CD27, CD70, CD40, CD40L, GITR, CD28, ICOS (CD278), ICOSL, VISTA and adenosine A2a receptor.

According to specific embodiments the immune modulatory agent is selected from the group consisting of a PD1 inhibitor, a PDL-1 inhibitor and a CTLA-4 inhibitor.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods for Examples 1-5

Plant growth and extract preparation—A high CBD strain of C. sativa IGB chemovar 10011 from the Israel Gene Bank (IGB) collection (GB10011), was cultivated from cuttings. For the vegetative phase, plants were grown for 10 weeks under long day conditions (18 h light/6 h dark). For the generative phase, the plants were exposed to short day conditions (12 h light/12 h dark) for 7 weeks. Inflorescence were harvested when the trichomes were mostly white. Frozen fresh/dry inflorescences were ground by mortar and pestle and placed in 15 mL tubes. Absolute ethanol was added to each inflorescence powder sample at a sample-to-absolute ethanol ratio of 1:4 (w/v). The samples were mixed thoroughly on a shaker for 30 min, and then the extract was syringe filtered (0.2 PVDF syringe filter). The filtrate was transferred to new tubes. The solvent was evaporated under nitrogen. The dried extract was weighed, and then resuspended in absolute methanol (volume of solvent added according to the desired concentration) and filtered through a 0.45 μm syringe filter. For the treatments, the resuspended extract was diluted according to cell cultures. Decarboxylation by heating the dry extract to 220° C. for 10 minutes. The dried extract was weighed, resuspended in absolute methanol (volume of solvent added according to the final desired concentration) and filtered through a 0.45 μm syringe filter.

Extract fractionation—Flash chromatography was carried out using a Buchi Pure C-810 Flash apparatus equipped with a diode array detector to separate the different cannabinoids from crude cannabis extracts, according to the following protocol:
Column: FP ECOFLEX C18 12 g
Flow Rate: 30 mL/min
Equilibration: 8.0 min
Mode: Flash Liquid
Solvent A: Water
Solvent B: Methanol
UV Threshold: 0.05 AU
UV Sensitivity: High
Gradient (water and methanol): 55% to 100%

Crude Cannabis extract was dissolved in a minimal amount of methanol and then filtered. Detection wavelengths were set at 220 and 280 nm and UV scan monitored from 200-400 nm. Based on the signal intensities of these two wavelengths, the system automatically collected fractions in fraction collector vials. Eluted fractions vials were divided into different fractions based on the identified peaks. The organic solvent (methanol) of each fraction was removed using a rotary vacuum evaporator. The remaining aqueous phases were further lyophilized for getting dried powder.

Standard/material preparation—Restek phytocannabinoid standards were used in this study: Δ-9 tetrahydrocannabinol (Δ-9 THC, 34067), cannabichromene (CBC, 34092), cannabidiol (CBD, 34011) at a concentration of 1 mg/mL, originally dissolved in methanol.

For quantification of phytocannabinoids by analytical HPLC, the standards were dissolved in methanol at various concentrations from 0-25 μg/mL. Mitomycin-C (MMC) was dissolved in water in stock concentration of 800 μg/mL Inverse agonists (IA) CB1 (AM251, Abcam ab120088), CB2 (SR144528, Abcam ab146185), TRPA1 blocker (HC-030031, Abcam ab120554) and TRPV1 and TRPV2 antagonists (Abcam ab141772 and Tranilast 1098/10, respectively) were dissolved in Dimethyl sulfoxide (DMSO) at a concentration of 10 mM.

Chemical analysis—High performance liquid chromatography (HPLC 1260 Infinity II, Agilent) analysis was done as described in using Isocratic separation with acenotrile (20%) and water with 5 mM ammonium formate, 0.1% formic acid (80%) at a constant flow rate of 1.5 mL/min. Gas chromatograph with mass selective detector (GC/MS 8860 GC/5977BMSD, Agilent) analysis was carried out as described in [34].

Cell cultures—UC cell line T24 (ATCC, HTB-4) was cultured in McCoy's 5A (BI-01-075-1A) growth media, while UC cell line 5637 (ATCC, HTB-9) was cultured in RPMI 1640 (BI-01-100-1A). Both contained 10% fetal bovine serum (FBA, BI-04-127-1A) and 1% pen-strep (BI-03-031-1B).

Cell proliferation assay—XTT assay was carried out as described in [34], on T24 and HTB-9 cells. 1*10⁴ cells/well were seeded in 96-wells plates, methanol was used as a vehicle control at the same concentration as the treatments. Mitomycin-C (MMC) was used as positive control. Cell viability was calculated relative to the vehicle control after subtracting blank.

Analysis of combined drug effects—Drug synergy was determined by the Bliss independence drug interaction model as described in [31], on T24 cells.

Apoptosis assay and cell cycle analysis—T24 cells were treated with cannabis compounds or with methanol as a vehicle control. Cell cycle was determined at 24 h and apoptosis was determined at 48 h post-treatment. Staining and detection followed manufacturer instructions [34]. Briefly, for the apoptosis assay, the MEBCYTO Apoptosis Kit with Annexin V-FITC and PI (MBL, Enco, 4700) was used. Cells were seeded in culture 6-wells plates, at density of 5×10⁵ cells per well in McCoy's 5A (for T24 a cells) or in RPMI (for HTB-9 cells). The following day, media was replaced with new media containing treatment or methanol control. Cells were then incubated for 24 or 48 h at 37° C. in a humidified 5% CO₂ 95% air atmosphere. Following incubation, cells were harvested and collected separately. Tubes were centrifuged for 2 min at 900 g and cell pellets were resuspended and washed twice with 1 mL of PBS. The cells in each sample were resuspended in 85 μL of Annexin binding buffer. Cells were stained using 10 μL of Annexin V-FITC solution and 5 μL of propidium iodide (PI) working solution followed by 15 min incubation in the dark at room temperature. Then 400 μL of Annexin V binding buffer were added to each tube and flow cytometry was performed using a FORTESA flow cytometer (FACS). Cells were considered to be apoptotic if they were Annexin V+/PI− (early apoptotic) or Annexin V+/PI+ (late apoptotic). Live cells were defined as Annexin V−/PI−, and Annexin V−/PI+ as necrotic.

For cell cycle analysis, cells were seeded in 6-wells culture plates at a concentration of 5×10⁵ cells per well in McCoy (for T24 cells). The following day, media was replaced with new media containing treatments or methanol control. Cells were then incubated for 24 h at 37° C. in a humidified 5% CO₂, 95% air atmosphere. Following 24 h of incubation, media was replaced with new media containing treatment or methanol control. Cells from each well were then harvested and collected separately and centrifuged for 2 min at 900 g. The cell pellet was washed once with 1 mL of PBS and fixed with 70% cold ethanol at 4° C. for at least 1 h. The fixed cells were pelleted out and washed twice with 1 mL of PBS. The cell pellet was then stained by re-suspending in 250 μL of PI solution (50 μg/mL) containing RNase A (100 μg/mL) for 30 min in the dark. Following, 200 μL of PBS were added to each tube and cells were analyzed using FACS.

Cell migration and cell invasion assays—For the cell migration assay T24 cells were seeded into a 96-wells tissue culture plate, 2×10⁴ cells per well. After 24 h the cell monolayer was scratched perpendicularly across the center of the well with a 200 μl pipette tip. Immediately following scratching, the culture medium was aspirated and 100 μl of treatment solution was added. Photos were taken at 0, 10, 12, 14 and 16 h following scratching, and the gap area was measured using ImageJ (rsb(dot)info(dot)nih(dot)gov/ij/download(dot)html) (n=12). The scratch area, indicated by cells migrated into the scratch at time x, was calculated as percent of scratch area at time x from time 0: (x h cell free area)*100/(0 h cell free area). A transwell assay, as described in [35], was used to determine cell invasion (n=3),

Cytoskeleton staining—T24 cell were seeded on glass bottom cell culture dishes and fixed with 3.7% formaldehyde solution and permeabilized with 0.1% Triton X-100 at room temperature. Fixed cells were blocked in Phosphate Buffered Saline (PBS) containing 1% bovine serum albumin. For actin and nuclear staining cells were incubated with EasyProbes ActinRed 555 Stain, and Hoechst, respectively. Image acquisition was effected using a Leica SP8 laser scanning microscope (Wetzlar, Germany), equipped with a 405 and 552 nm solid state lasers, HCX PL APO CS 10×/0.40 or HC PL APO CS 60×/1.2 water immersion objectives (Leica, Wetzlar, Germany) and Leica Application Suite X software (Wetzlar, Germany). Hoechst and ActinRed 555 emission signals were detected with PMT and HyD (hybrid) detectors in ranges of 415-490 and 565-660 nm, respectively. At least 3 images were captured from each slide, and experiments were repeated 4 times.

Quantitative real-time (qRT) PCR—qRT PCR was carried out as described in [34]. Briefly, T24 cells were treated with cannabis compounds or methanol as a vehicle control for 6 h. Cells were then harvested and total RNA was isolated and reverse-transcribed. The expression of each target gene was normalized to the expression of Hypoxanthine Phosphoribosyltransferase 1 (HPRT1; geneID 3251) mRNA as the 2^{-ΔΔCt} and is presented as the ratio of the target gene to HPRT1 mRNA, expressed as 2^{-ΔCt}, where Ct is the threshold cycle and ΔCt=Ct Target gene—Ct HPRT1. Experiments were repeated three times. The primers were: for CB1 (CNR1; geneID 1268) forward 5′-AAGACCCTGGTCCTGATCCT-3′ (SEQ ID NO: 1) and reverse 5′-TGTCGCAGGTCCTTACTCCT-3′ (SEQ ID NO: 2); for CB2 (CNR2; geneID 1269) forward 5′-ATCATGTGGGTCCTCTCAG-3′ (SEQ ID NO: 3) and reverse 5′-GATTCCGGAAAAGAGGAAGG-3′ (SEQ ID NO: 4).

Statistical analysis—Means of replicates were subjected to statistical analysis by Tukey-Kramer test or Student's t-test using the JMP statistical package and considered significant when P≤0.05.

### Materials and Methods for Examples 6-8

Plant growth and extract preparation—The inflorescences of a high CBD strain of C. sativa from IMC, super CBD (sCBD), and PARIS were extracted using ethanol, as previously described Frozen fresh/dry inflorescences were ground by mortar and pestle and placed in 15 mL tubes. Absolute ethanol was added to each inflorescence powder sample at a sample-to-absolute ethanol ratio of 1:4 (w/v). The samples were mixed thoroughly on a shaker for 30 min, and then the extract was syringe filtered (0.2 PVDF syringe filter). The filtrate was transferred to new tubes. The solvent was evaporated under nitrogen. The dried extract was weighed, and then resuspended in absolute methanol (volume of solvent added according to the desired concentration) and filtered through a 0.45 μm syringe filter. For the treatments, the resuspended extract was diluted according to cell cultures and decarboxylated by heating the dry extract to 220° C. for 10 minutes. The dried extracts were weighed, resuspended in absolute methanol (volume of solvent added according to the final desired concentration) and filtered through a 0.4511m syringe filter.

Extract fractionation—The GB10011 crude extract solution dissolved in methanol underwent fractionation process by a flash chromatography apparatus equipped with a diode array detector. An Ecoflex C-18 80g (Flash Pure, Buchi, C-18, 80 μm spherical, max. pressure 180 psi) column was used for separation, with gradient of methanol and water as the mobile phase, starting with 75% up to 100% methanol. Detection wavelengths were set at 220 and 280 nm and UV scan monitored from 200-400 nm. Based on the signal intensities of these two wavelengths, the system automatically collected fractions in fraction collector vials. Eluted fractions vials were divided into different fractions based on the identified peaks. Following collection of the fractions the organic solvent (methanol) was removed using a rotary vacuum evaporator at 30° C. The remaining aqueous phase containing the compound of interest was further lyophilized to precipitate a dried powder. Each dried fraction tube was weighed separately and reconstituted using methanol to produce a solution with the required concentration and stored at −20° C.

Standard/material preparation—Mitomycin C (MMC) was dissolved in water at a stock concentration of 800 μg/mL.

Chemical Analysis—High performance liquid chromatography analysis (HPLC 1260 Infinity II, Agilent) was carried out as described in using isocratic separation with acetonitrile (20%) and water with 5 mM ammonium formate and 0.1% formic acid (80%) at a constant flow rate of 1.5 ml/min.

Cell cultures—UC cell line T24 (ATCC, HTB-4) was cultured in McCoy 5A growth medium (BI-01-075-1A), while UC cell line HTB-9 (ATCC, 5637) was cultured in RPMI 1640 (BI-01-100-1A). Both contained 10% fetal bovine serum (FBA, BI-04-127-1A) and 1% pen-strep (BI-03-031-1B).

Biopsies Harvest and Treatment—Tissues from patients with urothelial carcinoma were obtained during transurethral tumor resection, at the Chaim Sheba Medical Center. All patients gave their informed consent according to IRP requirements. Tumors were cut to 250 μm thick slices using a vibratome (VF300, Precisionary Instruments, Boston 16 USA), placed on 24-well plates on titanium grids (Alabama R&D, Munford, USA) with 500 μl of DMEM/F12 medium (supplemented with 5 FCS, Penicillin 100 IU/ml with Streptomycin 100 μg/mL, Amphotericin B 2.5 μg/mL, Gentamicin sulfate 50 mg/ml and L-glutamine 100 Ml/mL). The tissues were cultured at 37° C., 5% CO2 and 80% O2, on an orbital shaker (TOU-120N, MRC) at 70 rpm. The following day, tissues were treated according to two treatment protocols: 1. Treatment exposure for 48 hours followed by drug change for another 48 hours. 2. Short-duration 2 hours treatment followed by wash and replacement of treatment with medium, repeated 3 times. Upon completion, tissues were fixed overnight with 4% PFA, followed by formalin-fixed paraffin embedding (FFPE). The blocks were cut and stained with hematoxylin and eosin and a blind pathological analysis was performed on the sections.

Cell proliferation assay—XTT assay was carried on T24 and HTB-9 cells. 1×10⁴ cells/well were seeded in 96-well plates. Methanol was used as vehicle control at the same concentration as the treatments, i.e. at concentrations that do not lead to cell death. MMC was used as a positive control. XTT reagent (2,3,-bis (2-methoxy-4-nitro-5-sulfophenyl)-5-[(phenylamino)-carbonyl]-2H-tetrazolium inner salt) was added for 2 h at 37° C. in a humidified atmosphere of 5% CO2 to 95% air. Absorbance was recorded by a Synergy 111 hybrid reader photometer (BioTek) at 490 nm with 650 nm of reference wavelength. Cell survival (% r, viability) was estimated using the equation: % cell survival=(A₄₉₀−A₆₅₀) of treatment/(A₄₉₀−A₆₅₀) of solvent control×100. A₄₉₀ and A₆₅₀ are the absorbencies of the XTT colorimetric reaction. The absorbance of medium alone (blank) was also reduced from specific readings. Apoptosis assay and cell cycle analysis—T24 cells were treated with cannabis compounds or with methanol as vehicle control. The cell cycle was determined at 24 hours and apoptosis was determined at 48 hours following treatment. Staining and detection followed the manufacturer's instructions. Briefly, for the apoptosis assay, a MEBCYTO apoptosis kit with Annexin V-FITC and propidium iodide (PI) (MBL, Enco, 4700) was used. Cells were seeded in 6-well plate culture dishes, at a density of 5×10⁵ cells per well. The following day, the media was replaced with new media containing treatments and vehicle control. Subsequently, cells were harvested and stained with 10 μL of Annexin V-FITC solution and 5 μL of PI working solution, flow cytometry performed using a FORTESA flow cytometer (FACS). Cells were considered apoptotic if they were Annexin V+/PI−(early apoptotic) or Annexin V+/PI+ (late apoptotic). Live cells were defined as Annexin V−/PI−, and Annexin V−/PI+ as necrotic. For determination of cell cycle phases, cells were seeded in 6-well plate culture dishes at a concentration of 5×10⁵ cells per well. Following 24 hours incubation, the medium was replaced with new medium containing treatments and vehicle control. The cells of each well were then harvested and fixed with 70% cold ethanol at 4° C. for at least 1 hour. The fixed cells were then pelleted and washed twice with 1 ml of PBS. Following, the cell pellet was stained by re-suspending in 250 μL of PI solution (50 μg/mL) containing RNase A (100 μg/mL) for 30 minutes in the dark. Then 200 μL of PBS was added to each tube and the cells were analyzed using FACS.

Cell migration and cell invasion assays—For the cell migration assay, T24 cells were seeded in a 96-well tissue culture plate, 2×10⁴ cells/well. Following 24 hours, the cell monolayer was scratched perpendicularly across the center of the well with a 200 μL pipette tip. Immediately after scratching, the culture medium was aspirated and 100 μL of treatment solution was added. Photos were taken at 0, 10, 12, 14 and 16 hours after scratching, and the gap area was measured using ImageJ (rsb.info.nih.gov/ij/download.html) (n=12). The scratch area, indicated by the cells that migrated to the scratch at time x, was calculated as the percentage of the scratch area at time x from time 0:

$$\frac{( x is cell free area ) \times 100}{( 0 h cell free area )}$$

Quantitative real-time (qRT) PCR—T24 cells were treated with cannabis compounds or methanol as a vehicle control for 6 hours. Cells were then harvested and total RNA was isolated. The RNA was reverse transcribed. The expression of each target gene was normalized to the expression of hypoxanthine phosphoribosyltransferase 1 (HPRT1; geneID 3251) mRNA as the 2^{-66 ΔCt} method that presents the differences ( ) in the threshold cycle (Ct) between the target gene and the HPRT1 gene. ΔCt=Ct target gene—Ct HPRT1. Experiments were repeated 4 times (4 biological repeats), with two more technical repeats. The primers were as follows:

| | for caspase 3 forward |
|---|---|
| (SEQ ID NO: 5) | |
| | 5′-GAGGCCGACTTCTTGTATGC-3′ |
| | and |
| | reverse |
| (SEQ ID NO: 6) | |
| | 5′-CGGTTAACCCGGGTAAGAAT-3′; |
| | for caspase 7 |
| | forward |
| (SEQ ID NO: 7) | |
| | 5′-GAAGAGGCTCCTGGTTTGTG-3′ |
| | and |
| | reverse |
| (SEQ ID NO: 8) | |
| | 5′-TCTCATGGAAGTGTGGGTCA-3′; |
| | for E-cadherin |
| | forward |
| (SEQ ID NO: 9) | |
| | 5′-TGGAGAGACACTGCCAACTG-3′ |
| | and |
| | reverse |
| (SEQ ID NO: 10) | |
| | 5′-TTAGGGCTGTGTACGTGCTG-3′; |
| | for N-cadherin |
| | forward |
| (SEQ ID NO: 11) | |
| | 5′-GGATCAACCCCATACACCAG-3′ |
| | and |
| | reverse |
| (SEQ ID NO: 12) | |
| | 5′-TGGTTTGACCACGGTGACTA-3′. |

Sphere Formation and Self-Renewal Assay—Cells were seeded in a 24-well plate, 500,000 cells per well in 500 μl McCoy (normal+serum medium) and incubated for 24 hours. The culture medium was aspirated, and treatment solution was added. Following 24 hours, treatment solutions were aspirated and pre-warmed medium culture was added again for another 24 hours. On the fourth day, cells were suspended using a 150 l trypsin solution per well. Two centrifuge cycles at 1200 rpm, 1 minute each, were performed to replace media and trypsin with serum free culture medium (DMEM-F12) enriched with EGF, FGF, and IGF (PeproTech, cat no. AF-100-15, 100-18B-10, 100-11-100), at concentration of 20 ng/ml). The cells were then transferred to ultralight attachment plates and maintained with serum-free medium (DMEM-F12, serum-free) enriched with growth factors (EGF, b-FGF, and IGF-1). The mean sphere size of T24 and the total number of spheres per well were measured at time points 0, 24, 72, 96 and 120 hours. After sphere creation, cells were transferred to McCoy culture conditions (also referred to as normal culture conditions) in a 12-well plate and serum-containing culture medium (McCoy). The patches of colonies of UC cells were evaluated by taking pictures using an inverted microscope (ZEISS), after 36 hours and counting spheres using IMAGJ software.

Statistical analysis—The mean replicates were subjected to statistical analysis using the Tukey-Kramer test or Student's t test using the JMP statistical package (www(dot)jmp(dot)com/en_us/home(dot)html, SAS Inc., NC, USA) and considered significant when P<0.05.

### Example 1

### C. sativa Extracts and Chromatography Fraciotnated Fractions have Cytotoxic Activity Against Urothelial Cell Carcinoma (UCC)

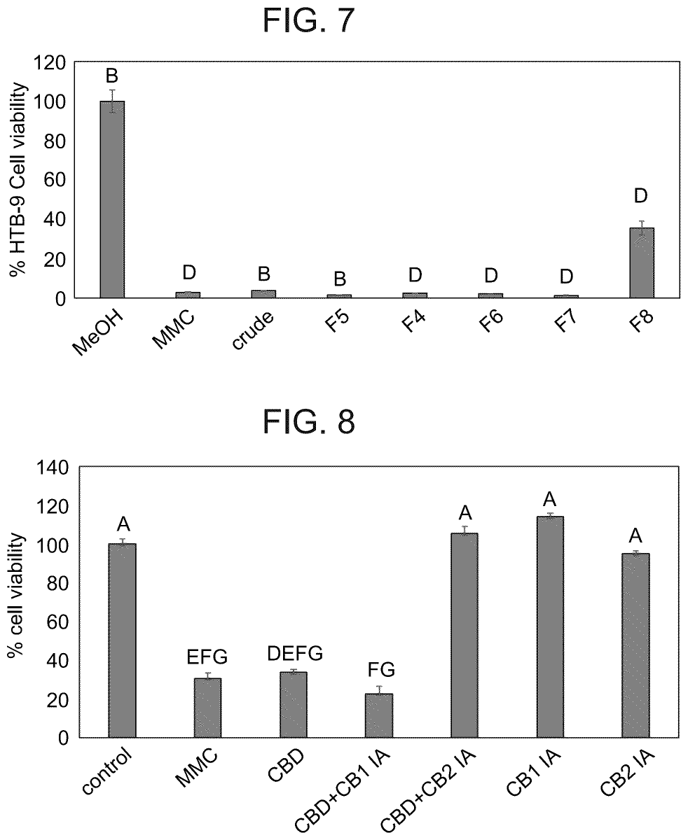

Ethanol extract of fresh inflorescence of a high-CBD cannabis strain from IGB (GB10011) was fractionated using Flash chromatography (FIG. 1A). The crude extract showed a cytotoxic activity against UC cell line T24 (FIG. 1B). Some of the extract fractions demonstrated a significant cytotoxic activity in comparison to the vehicle control, with some (i.e. GB10011-F4, GB10011-F6 and GB10011-F7) demonstrating a significantly increased activity in comparison to 8 μg/mL MMC (FIG. 1B) while others (i.e. GB10011-F8) had only a minor cytotoxic activity (FIG. 1B). The IC50 of GB10011-F4, GB10011-F5, GB10011-F6 and GB10011-F7 of GB10011 were 13.38, 21.89, 20.25 and 13.05 μg/mL, respectively (FIGS. 1C-F). GB10011-F4-GB10011-F7 were also highly active on another UC cell line, HTB-9 (FIG. 7).

### Example 2

### The Chemical Composition of C. sativa Extracts and Chromatography Fractionated Fractions

Based on HPLC and GC/MS analysis, the crude extract contained approximately 5-10% phytocannabinoids by total content and each of fractions GB10011-F4-GB10011-F7 contained approximately 60-80% phytocannabinoids by total content. Following, the most active fraction GB10011-F7 was subjected to chemical analysis. GB10011-F7 contained CBC (85.8%) and THC (14.2%) out of the total phytocannabinoids. In comparison, the crude extract contained 3.0% CBC and 26.4% THC out of the total phytocannabinoids (Table 1 hereinbelow). The phytocannabinoid composition of the other fractions GB10011-F4-GB10011-F6 is also presented (Table 1 hereinbelow). In addition, GC/MS analyses showed no terpenes in GB10011-F7.

TABLE 1
Phytocannabinoid composition of the crude extract of GB10011 and
chromatography fractionated fractions, based on HPLC analysis.

| | | % from |
|---|---|---|
| | Phytocannabinoid | phytocannabinoid |
| Crude | CBDVA | 4.6 |
| extract | CBG | 4.7 |
| | CBD | 50.7 |
| | THCV | 0.1 |
| | CBN | 0.5 |
| | THC | 26.4 |
| | CBC | 3.0 |
| GB10011- | CBG | 4.4 |
| F4 | CBD | 95.4 |
| | THCV | 0.1 |
| GB10011- | CBD | 12.7 |
| F5 | CBDA | 2.4 |
| | THC | 51.3 |
| | THCA | 7.3 |
| | CBDV | 0.8 |
| | THCV | 2.6 |
| | CBG | 3.7 |
| | CBN | 18.4 |
| GB10011- | CBG | 0.35 |
| F6 | CBD | 0.2 |
| | CBN | 0.64 |
| | THC | 98.8 |
| GB10011- | CBC | 85.8 |
| F7 | THC | 14.2 |

### Example 3

### Combinations of Pure Cannabinoids Mimicking Chromatography-Fractionated Fraction GB10011-F7 have Cytotoxic Activity Against UCC

A combination of pure CBC and THC standards at the ratio found in GB10011-F7 (˜6:1) was active at a level similar to GB10011-F7 (IC50 of 13.68 and 13.05, respectively; FIGS. 1F-G). In addition, a combination of pure CBC and THC standards at the ratio found in GB10011-F7 (˜6:1) had a similar cytotoxic activity on HTB-9 (IC50 of 13.95 μg/mL; FIG. 1H).

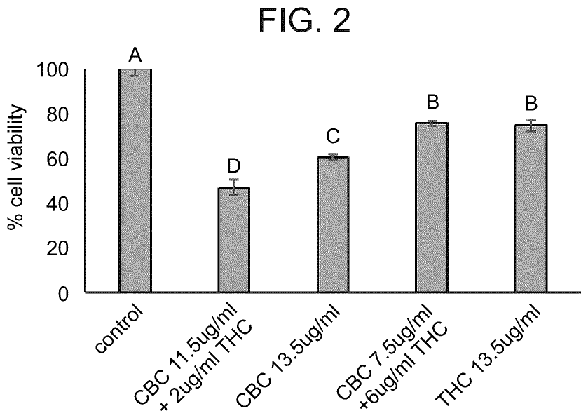

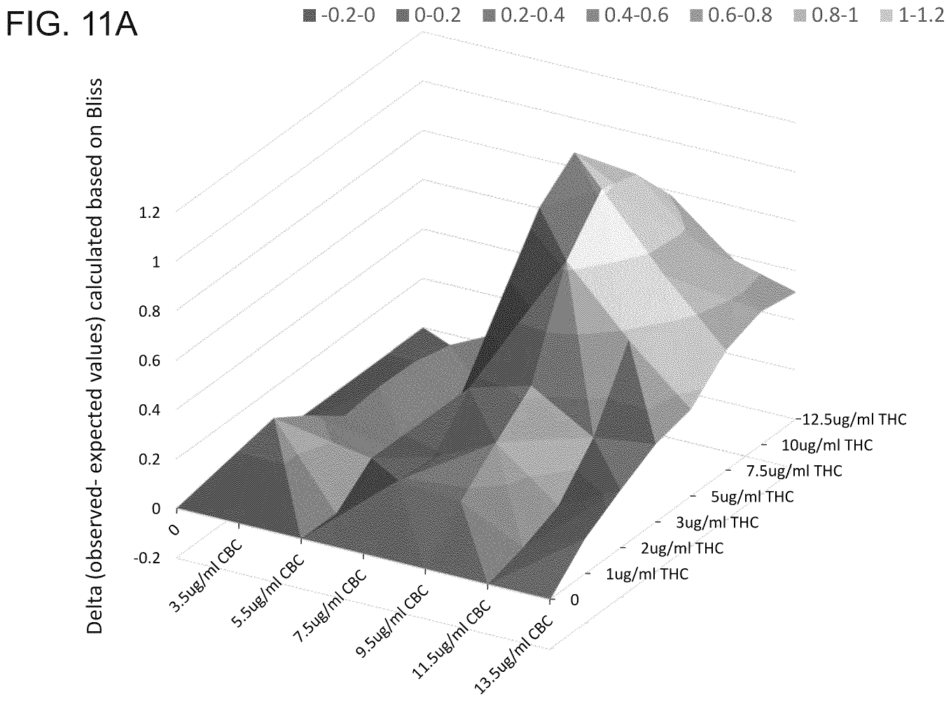

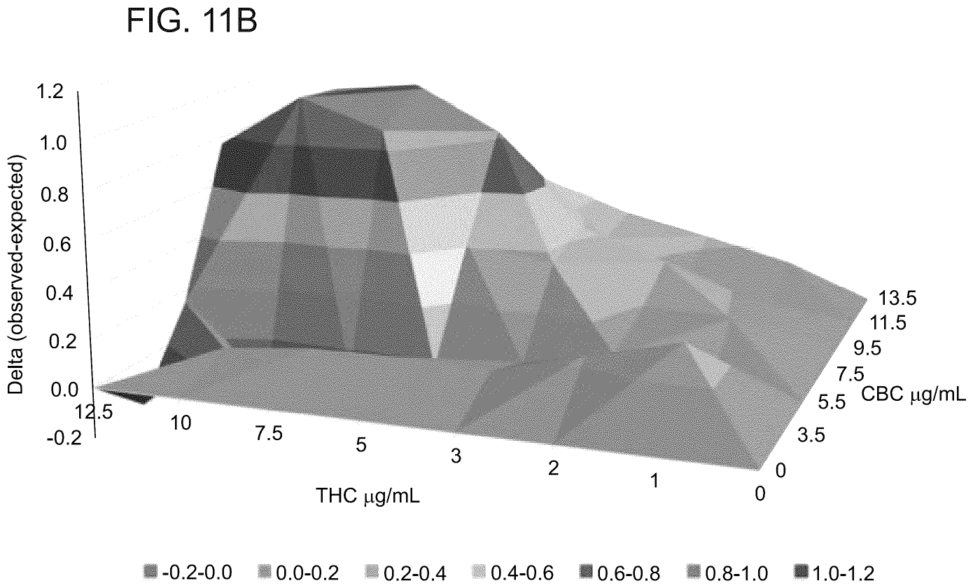

In the next step, the synergistic interaction on T24 cell viability between the two GB10011-F7 compounds, CBC and THC, was determined using the Bliss independence-drug interaction-model. Tables 2-3 hereinbelow and FIG. 11 demonstrate synergistic activity using CBC and THC in several concentrations and ratios. Furthermore, the combination of CBC and THC at the approximate CBC:THC ratio (˜6:1) identified in GB10011-F7 (11.5+2.0 μg/mL, respectively) was more effective than CBC or THC only at a concentration of 13.5 μg/mL (FIG. 2).

TABLE 2
Concentrations and ratios of CBC and THC demonstrating synergistic activity against
T24 cell viability

| CBC | | | | | | | | |
|---|---|---|---|---|---|---|---|---|
| (μg/ | THC (μg/ml) | | | | | | | |
| ml) | 1 | 2 | 3 | 4 | 5 | 6 | 7 | 8 |
| 1 | | | | | | | | |
| 2 | | | | | | | | |
| 3 | | | | | | | | |
| 4 | | | | | | | | |
| 5 | | | | | | | | |
| 6 | | | | | | | | |
| 7 | | | | | | | | |
| 7.5 | | 7.5:2 | 7.5:3 | 7.5:4 | 7.5:5 | 7.5:6 | 7.5:7 | 7.5:8 |
| 8 | | 8:2 | 8:3 | 8:4 | 8:5 | 8:6 | 8:7 | 8:8 |
| 9 | | 9:2 | 9:3 | 9:4 | 9:5 | 9:6 | 9:7 | 9:8 |
| 10 | | 10:2 | 10:3 | 10:4 | 10:5 | 10:6 | 10:7 | 10:8 |
| 11 | | 11:2 | 11:3 | 11:4 | 11:5 | 11:6 | 11:7 | 11:8 |
| 11.5 | | 11.5:2 | 11.5:3 | 11.5:4 | 11.5:5 | 11.5:6 | 11.5:7 | 11.5:8 |
| 12 | | 12:2 | 12:3 | 12:4 | 12:5 | 12:6 | 12:7 | 12:8 |
| 13 | | 13:2 | 13:3 | 13:4 | 13:5 | 13:6 | 13:7 | 13:8 |
| 14 | | 14:2 | 14:3 | 14:4 | 14:5 | 14:6 | 14:7 | 14:8 |
| 15 | | 15:2 | 15:3 | 15:4 | 15:5 | 15:6 | 15:7 | 15:8 |
| CBC | | | | | | | | |
| (μg/ | | | | | | | | |
| ml) | 9 | 10 | 11 | 12 | 13 | 13.5 | 14 | 15 |
| 1 | | | | | | 1:13.5 | 1:14 | 1:15 |
| 2 | | | | | | 2:13.5 | 2:14 | 2:15 |
| 3 | | | | | | 3:13.5 | 3:14 | 3:15 |
| 4 | | | | | | 4:13.5 | 4:14 | 4:15 |
| 5 | | | | | | 5:13.5 | 5:14 | 5:15 |
| 6 | | | | | | 6:13.5 | 6:14 | 6:15 |
| 7 | | | | | 7:13 | 7:13.5 | 7:14 | 7:15 |
| 7.5 | 7.5:9 | 7.5:10 | 7.5:11 | 7.5:12 | 7.5:13 | 7.5:13.5 | 7.5:14 | 7.5:15 |
| 8 | 8:9 | 8:10 | 8:11 | 8:12 | 8:13 | 8:13.5 | 8:14 | 8:15 |
| 9 | 9:9 | 9:10 | 9:11 | 9:12 | 9:13 | 9:13.5 | 9:14 | 9:15 |
| 10 | 10:9 | 10:10 | 10:11 | 10:12 | 10:13 | 10:13.5 | 10:14 | 10:15 |
| 11 | 11:9 | 11:10 | 11:11 | 11:12 | 11:13 | 11:13.5 | 11:14 | 11:15 |
| 11.5 | 11.5:9 | 11.5:10 | 11.5:11 | 11.5:12 | 11.5:13 | 11.5:13.5 | 11.5:14 | 11.5:15 |
| 12 | 12:9 | 12:10 | 12:11 | 12:12 | 12:13 | 12:13.5 | 12:14 | 12:15 |
| 13 | 13:9 | 13:10 | 13:11 | 13:12 | 13:13 | 13:13.5 | 13:14 | 13:15 |
| 14 | 14:9 | 14:10 | 14:11 | 14:12 | 14:13 | 14:13.5 | 14:14 | 14:15 |
| 15 | 15:9 | 15:10 | 15:11 | 15:12 | 15:13 | 15:13.5 | 15:14 | 15:15 |

TABLE 3
Concentrations and ratios of CBC and THC demonstrating synergistic activity against
T24 cell viability. Synergy is apparent when the experimental (observed) value of cell
survival inhibition is higher than the calculated (expected) value, hence DELTA > 0

| | | CBC [μg/ml] | | | | | | |
|---|---|---|---|---|---|---|---|---|
| | | 0 | 3.5 | 5.5 | 7.5 | 9.5 | 11.5 | 13.5 |
| | Calculated (expected) values* | | | | | | | |
| THC | 12.5 | 0.270064 | 0.079022 | 0.272975 | 0.229461 | 0.225539 | 0.418097 | 0.490861 |
| [μg/ml] | 10 | 0.042333 | −0.20831 | 0.046153 | −0.01094 | −0.01608 | 0.236551 | 0.463822 |
| | 7.5 | −0.01826 | −0.28477 | −0.0142 | −0.0749 | −0.08037 | 0.188244 | 0.443347 |
| | 5 | 0.048805 | −0.20015 | 0.052599 | −0.00411 | −0.00922 | 0.24171 | 0.501148 |
| | 3 | −0.01374 | −0.27905 | −0.00969 | −0.07012 | −0.07557 | 0.191854 | 0.529166 |
| | 2 | −0.03207 | −0.30218 | −0.02795 | −0.08948 | −0.09502 | 0.17724 | 0.531028 |
| | 1 | −0.01141 | −0.27612 | −0.00737 | −0.06767 | −0.0731 | 0.193709 | 0.512708 |
| | 0 | −1E−10 | −0.26172 | 0.003989 | −0.05563 | −0.061 | 0.202803 | 0.508789 |
| | Experimental (observed) values | | | | | | | |
| | 12.5 | 0.270064 | −0.11531 | 0.419625 | 0.998125 | 1.005343 | 0.997743 | 1.003516 |
| | 10 | 0.042333 | −0.11367 | −0.11887 | 0.988333 | 0.956144 | 0.988265 | 1.002734 |
| | 7.5 | −0.01826 | −0.15063 | −0.11528 | 0.804375 | 0.935218 | 0.918369 | 0.928906 |
| | 5 | 0.048805 | −0.06198 | 0.032509 | 0.412083 | 0.820125 | 0.813166 | 0.85332 |
| | 3 | −0.01374 | −0.15601 | 0.031911 | 0.28875 | 0.35463 | 0.47394 | 0.845313 |
| | 2 | −0.03207 | −0.07274 | 0.089749 | 0.094167 | 0.208816 | 0.286006 | 0.765234 |
| | 1 | −0.01141 | 0.047012 | −0.01097 | 0.12625 | 0.096839 | 0.238274 | 0.65625 |
| | 0 | −1E−10 | −0.26172 | 0.003989 | −0.05563 | −0.061 | 0.202803 | 0.508789 |
| | Delta between observed and experimental values calculated according to Bliss model | | | | | | | |
| | 12.5 | 0 | −0.19433 | 0.14665 | 0.768664 | 0.779803 | 0.579646 | 0.512655 |
| | 10 | 0 | 0.09464 | −0.16502 | 0.999271 | 0.972226 | 0.751714 | 0.538913 |
| | 7.5 | 0 | 0.134141 | −0.10108 | 0.879279 | 1.015593 | 0.730125 | 0.48556 |
| | 5 | 0 | 0.138165 | −0.02009 | 0.416189 | 0.82934 | 0.571455 | 0.352173 |
| | 3 | 0 | 0.123049 | 0.041602 | 0.358874 | 0.430201 | 0.282086 | 0.316146 |
| | 2 | 0 | 0.229443 | 0.117698 | 0.183642 | 0.303835 | 0.108766 | 0.234207 |
| | 1 | 0 | 0.32313 | −0.0036 | 0.193918 | 0.16994 | 0.044566 | 0.143542 |
| | 0 | 0 | 1.26E−10 | 9.96E−11 | 1.06E−10 | 1.06E−10 | 7.97E−11 | 4.91E−11 |

*expected inhibition was calculated according to Bliss model − Fi(x)+Fi(y) − Fi(x)*Fi(y). [Fi(x) − inhibition at concentration x]

### Example 4

### The Involvement of the Cannabinoid Receptors in the Cytotoxic Activity Against UCC

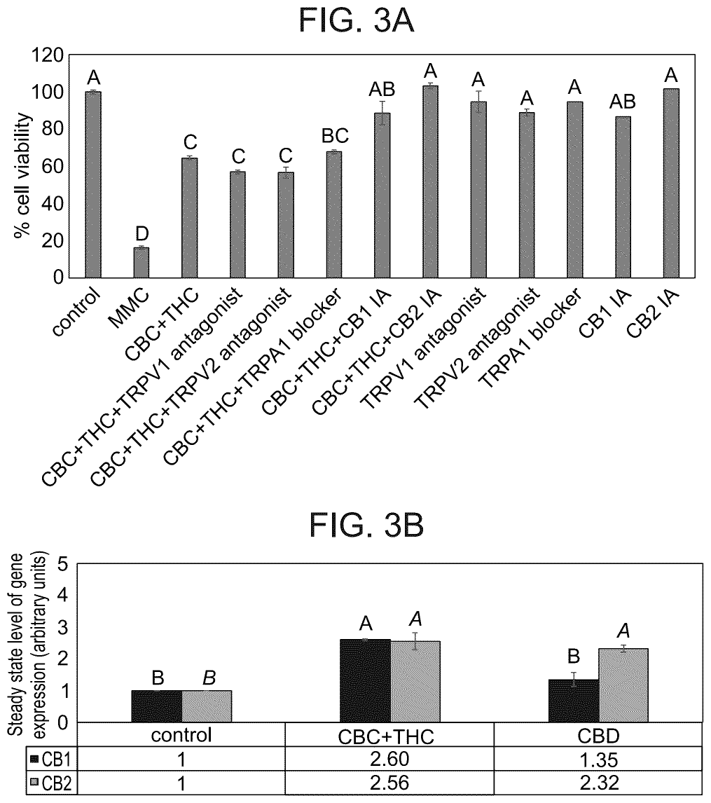

T24 cells were treated with a mixture of pure CBC and THC standards at the ratio found in GB10011-F7 (11.5+2.0 μg/ml, respectively) with and without a CB1 or CB2 inverse agonist, a TRPV1 or TRPV2 antagonist or a TRPA1 blocker. Addition of a CB1 or CB2 inverse agonist significantly decreased the cytotoxic activity of the CBC+THC composition (FIG. 3A). However, addition of a TRPA1 blocker or a TRPV1 or TRPV2 antagonist did not significantly affect the cytotoxic activity of the CBC+THC composition (FIG. 3A). Treatment with the inverse agonists, antagonists or blocker only did not have a significant effect on cell viability (FIG. 3A). CBD is known for its anti-cancer activity and is the abundant phytocannabinoid in the IGB strain (50.7%, see Table 1 above). Hence, the effect of CB1 and CB2 inverse agonists on CBD activity was determined. Only CB2 inverse agonist completely abolished cytotoxicity of CBD against T24 cells (FIG. 8).

Both CB1 and CB2 receptor genes (i.e., CNR1 and CNR2, respectively) are expressed at the RNA level in T24 cells, as determined with HPRT1 as a reference gene. CB2 receptor RNA level was induced 2.56-fold and CB1 receptor RNA level was induced 2.6-fold following treatment with CBC+THC (FIG. 3B). RNA levels of both CB1 and CB2 receptors were increased by 1.35 and 2.32-fold, respectively, following treatment with CBD (FIG. 3B). Similar patterns of expression were obtained with actin as the reference gene (not shown).

### Example 5

### Combinations of Pure Cannabinoids Mimicking Fraction GB10011-F7 Induce UCC Cell Cycle Arrest and Apoptosis, Reduce Cell Invasion And Disintegrate F-Actin

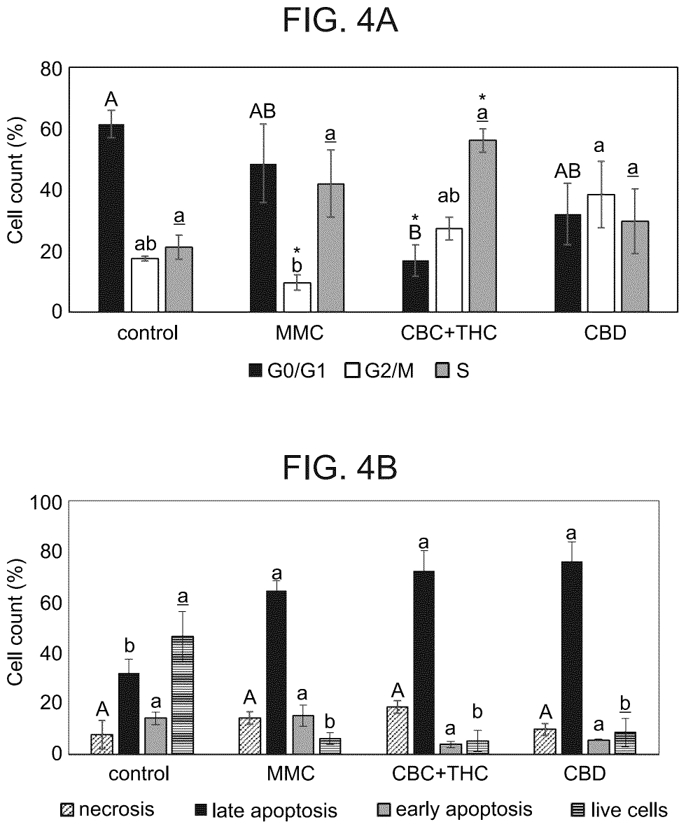

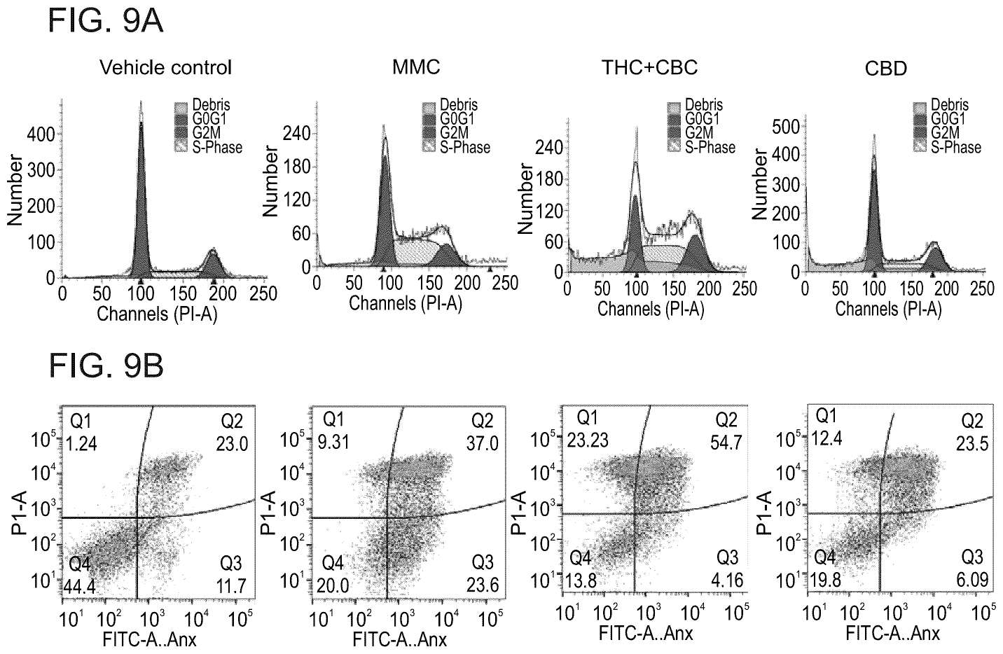

A mixture of pure CBC and THC standards at the ratio found in GB10011-F7 (17.2+2.8 μg/ml, respectively) led to a significant reduction in cells in G2/M-phase and to a significant enrichment in the percentage of cells in the S-phase of the cell cycle (56.0%, FIGS. 4A and 9A-B). This is in comparison to 21.2% S-phase cells in the control (FIG. 4A). CBD treatment led to an insignificant increase in G2/M-phase cells (38.3%) in comparison to 17.4% in the control (FIG. 4A).

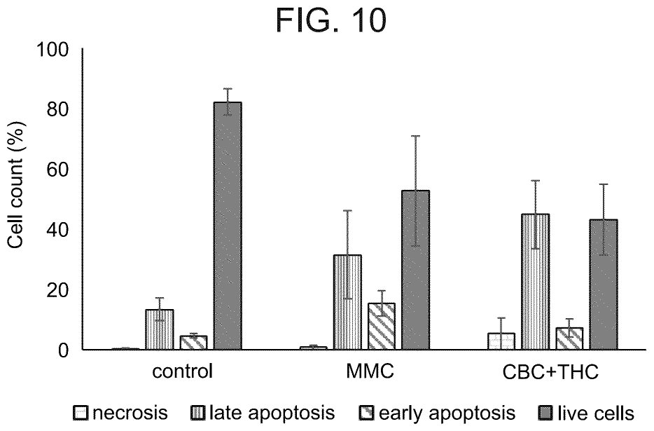

48 hours treatment with CBC+THC led to 76.2% cell apoptosis in comparison to 46.2% in the control and 79.6% in MMC-treated cells (FIGS. 4B and 9A-B). The proportion of apoptotic cells in the CBD-treated cells was higher (81.7%). All examined treatments led to a minor increase in necrosis in comparison to the control (7.6%), with CBC+THC treatment resulting in the highest level of necrosis (18.5%; FIGS. 3B and 10). An increase in apoptosis with the CBC+THC treatment was evident already at 24 hours (FIG. 10).

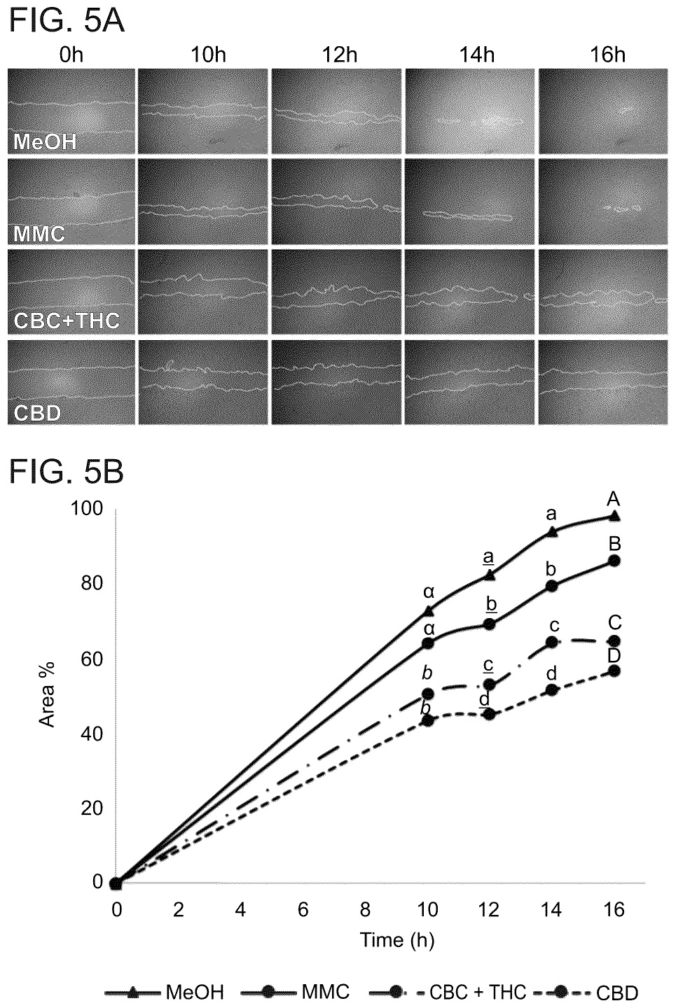

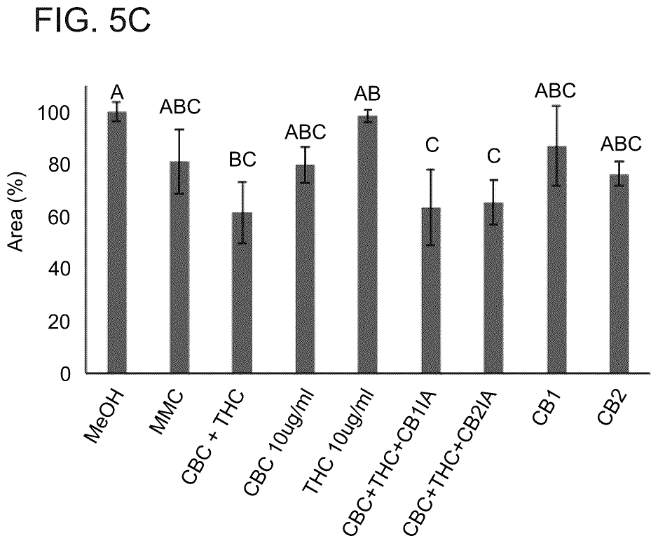

Following, the effect of a combination of pure CBC and THC standards at the ratio found in GB10011-F7 or CBD on cell migration was examined in scratch assays with treatments at sub-lethal concentrations (i.e. CBD at a concentration of 10 μg/mL; CBC+THC concentrations 8.6+1.4 μg/mL, respectively); MMC at a concentration of 4 μg/mL) (FIGS. 5A-C). A marked and significant reduction in the ability of the cells to migrate into the scratch was noted for cells treated with CBD or CBC+THC in comparison to the control (FIGS. 5A-B). MMC treatment was effective in reducing cell migration in this assay in comparison to the control, but to a lesser extent than CBD or CBC+THC treatments (FIGS. 5A-B). CBC at concentration of 10 μg/mL reduced cell migration to a lesser extent than treatment with CBC+THC, whereas THC 10 μg/mL had no effect on cell migration (FIG. 5C). Addition of CB1 or CB2 inverse agonists did not affect the CBC+THC reduction of cell motility (FIG. 5C).

Further, the effect of a combination of pure CBC and THC standards at the ratio found in GB10011-F7 or CBD on cell invasion was examined using the transwell assay using the above indicated sub-lethal concentrations. CBC+THC or CBD treatment reduced cell invasion only to a minor extent (90.5±4.1% or 89.7±6.2%, respectively) relative to a vehicle control (Table 4 hereinbelow). MMC treatment as a positive control reduced cell invasion by 43.7±4.8% in comparison to control (Table 4 hereinbelow).

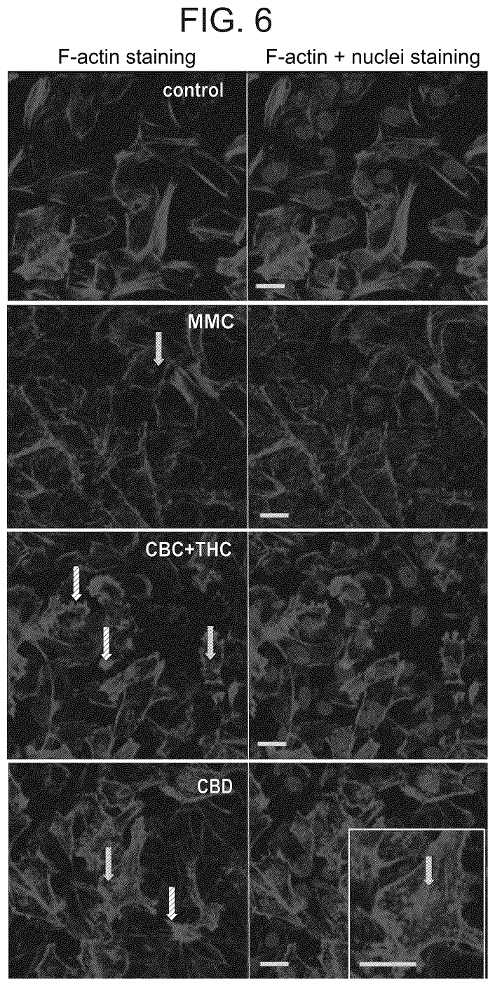

Treatment with the sub-lethal concentrations of CBC+THC led to disintegration of F-actin filaments visualized as characteristic spots (FIG. 6, yellow arrows; [30]) and partial disappearance of F-actin filaments inside the cells (FIG. 6). Similar effects on F-actin organization was detected for CBD and MMC treatments (FIG. 6, yellow arrows), whereas actin filaments were intact in the vehicle control group (FIG. 6). In addition, CBC+THC and CBD treatments also induced accumulation of F-actin filaments in the cell periphery (FIG. 6, white arrows).

TABLE 4
The effect of the cannabinoid compositions on cell invasion

| | Percentage of cell invasion |
|---|---|
| Treatment | relative to vehicle control |
| MMC 4 μg/mL | 43.7 ± 4.8 |
| CBC + THC (8.6 + 1.4 μg/mL) | 90.5 ± 4.1 |
| CBD (10 μg/mL) | 89.7 ± 6.2 |

### Example 6

### Synergy Between a Combinations of Pure Cannabinoids Mimicking Fraction GB10011-F7 and MMC

A synergistic activity between the mixture of pure CBC and THC standards at the ratio found in GB10011-F7 and MMC was detected on T24 cell viability, using the Bliss independence drug interaction model (FIG. 15). The outcome of the Bliss model represents the delta between the experimental survival analysis (i.e., fraction of inhibition achieved by the combination of drugs) and the calculated (expected) fraction of inhibition, based on cell survival analysis of CBC+THC only composition or MMC alone. A higher delta between the observed effect and the calculated effect represents a more profound synergy achieved by the combined CBC+THC only composition with MMC.

### Example 7

### C. sativa Extracts, Chromatography Fraciotnated Fractions and Combinations of Pure Cannabinoids Mimicking Same have Cytotoxic Activity Against UC

Ethanol extracts of the commercial strains, DQ (Dairy Queen, IMC, Israel), sCBD (Super CBD, IMC, Israel) and PARIS were obtained. The chemical composition of the crude extracts is shown in Table 6 hereinbelow. Of note, the total concentration of phytocannabinoids was higher in the DQ extract (82.75%) compared to the sCBD extract (66.4%).

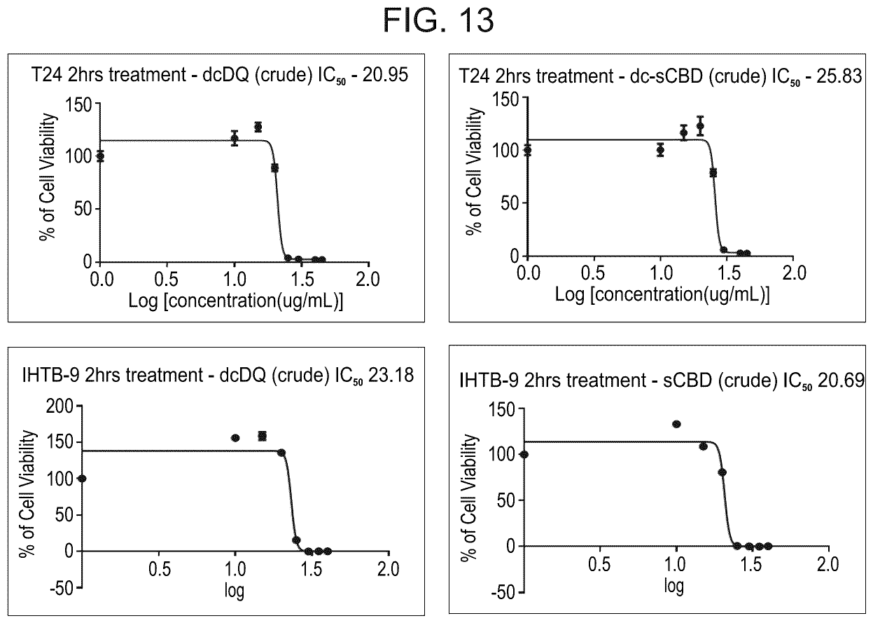

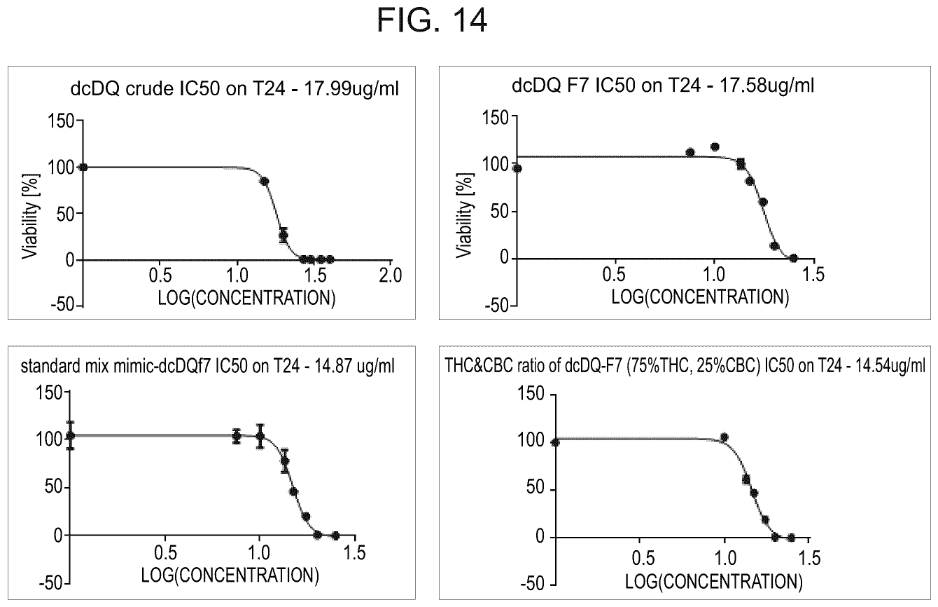

Following, the cytotoxic activity of the whole DQ and sCBD extracts against two distinct UC cell lines (T24 and HTB9) was evaluated, following a 2 or 48 hours exposure protocol. Both the DQ and sCBD extracts had a significant, dose dependent cytotoxic activity (FIGS. 12-13).

In addition the DQ extract was fractionated using flash chromatography. A fraction, referred to herein as “DQ F7” has a significant, dose dependent cytotoxic activity against T24 UC cells (FIG. 14). The chemical composition of DQ F7 is shown in Table 6 hereinbelow.

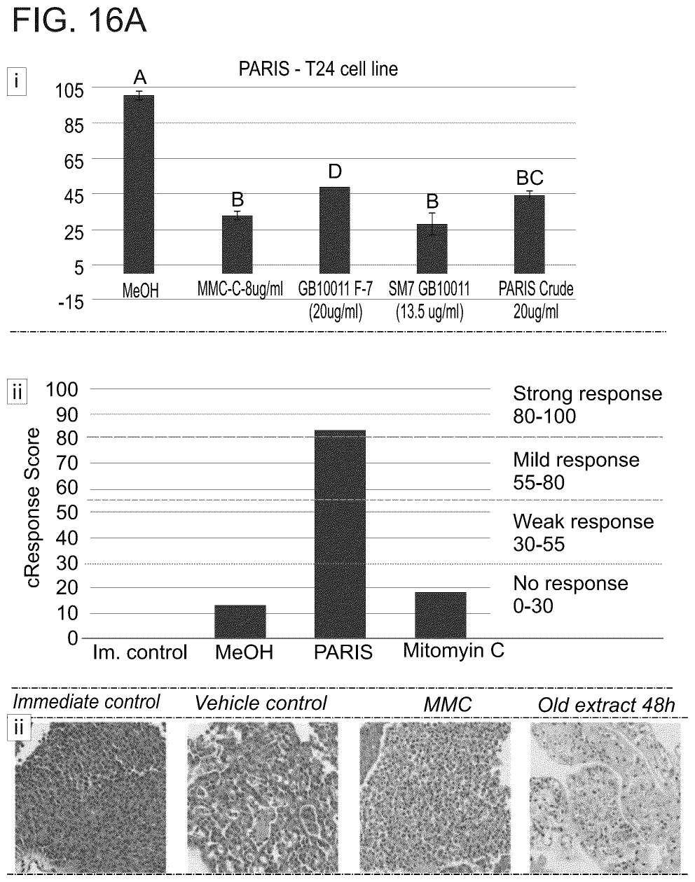

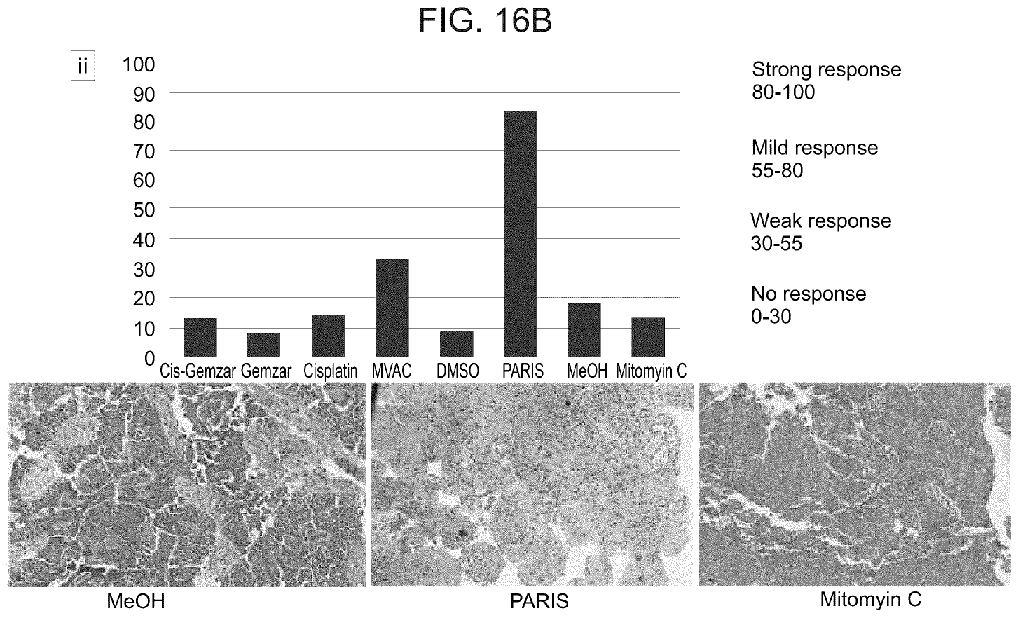

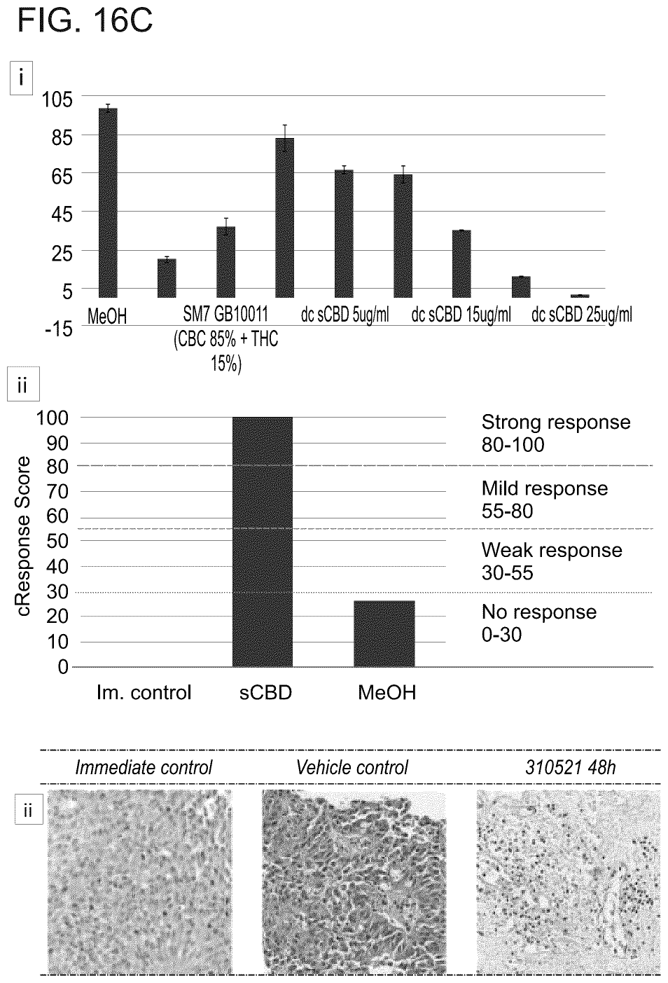

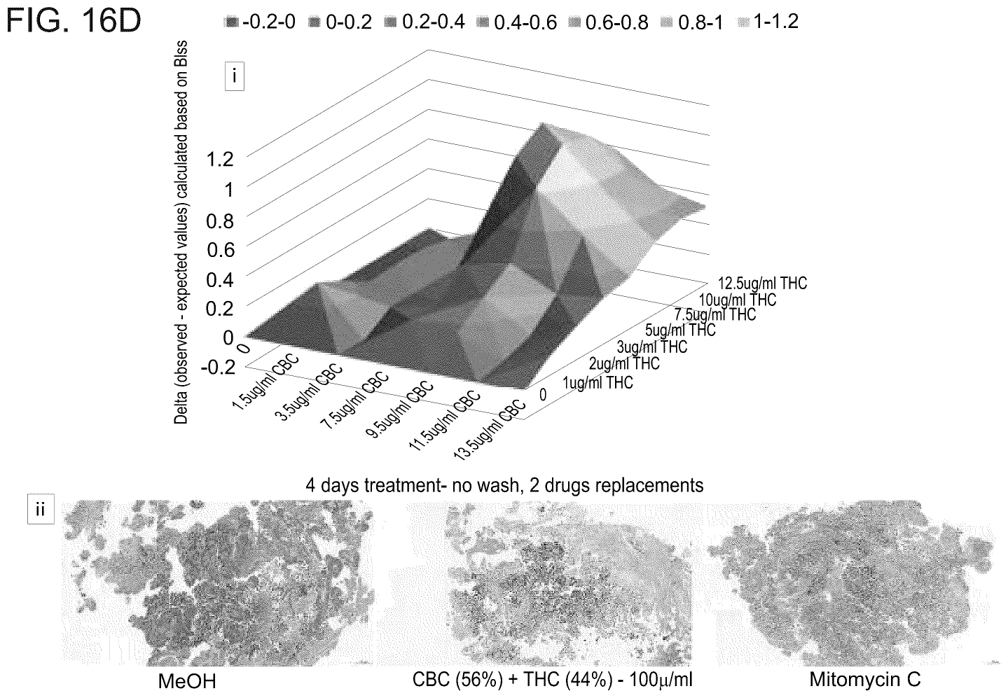

In the next step, tissues obtained from bladder UC patients during transurethral resection of tumor were cultured using ex-vivo organ culture technique. Tumors were treated with the crude extracts of sCBD or PARIS. Treatment protocol included both continuous exposure and 2 hours of repeated exposure. Both protocols exhibited a profound cytotoxic effect of all three extracts (FIGS. 16A-C).

Further, pure cannabinoids were used to obtain a standard composition mimicking DQ F7 and a composition comprising CBC and THC only in a ratio similar to DQ F7 (i.e. CBC 25%, THC 75%). Both compositions had a significant dose dependent cytotoxic activity against T24 UC cells (FIG. 14).

TABLE 6
Phytocannabinoid percentage of total phytocannabinoids
in the indicated extracts and fractions.

| | | % from |
|---|---|---|
| | Phytocannabinoid | phytocannabinoid |
| DQ | CBC | 5 |
| Crude | CBD | 0.1 |
| extract | CBDV | <0.1 |
| | CBDVA | <0.1 |
| | GBG | 4.0 |
| | CBGA | 0.1 |
| | CBN | 1.8 |
| | THC | 87.4 |
| | THCA | 0.5 |
| | THCV | 1.0 |
| DQ F7 | CBC | 23.5 |
| | CBDV | 1.8 |
| | CBDVA | 0.9 |
| | CBG | 1.3 |
| | CBN | 1.9 |
| | THC | 68.9 |
| | THCA | 1.7 |
| sCBD | CBD | 88.7 |
| extract | THC | 1.77 |
| | CBG | 3.49 |
| | CBC | 4.64 |
| | CBDV | 1.02 |
| | CBDA | 0.34 |
| PARIS | CBC | 2.68 |
| extract | CBD | 58.82 |
| | CBG | 3.37 |
| | THC | 35.1 |

### Example 8

### DQ and SCBD Extracts Induce UC Cell Cycle Arrest and Apoptosis, Reduce Cell Invasion, Inhibit Sphere Formation and Epithelial to Mesenchymal Transition

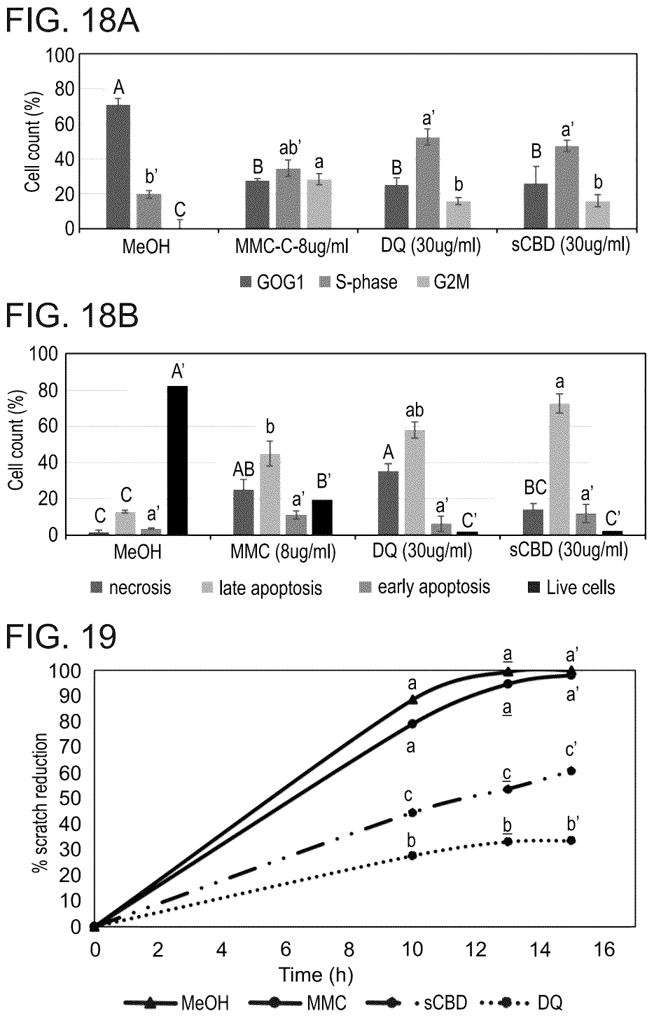

Treatment with a DQ and sCBD crude extract led to enrichment of T24 S-phase cells (52.3 and 47.2% for DQ and sCBD, respectively) in comparison to vehicle control (19.6%), whereas MMC led to 34.4% of S-phase cells (FIG. 18A). Cell population treated with DQ and sCBD crude extracts was also enriched for G2/M-phase cells (15.6 and 15.8%, respectively; FIG. 18A). MMC led to 28.1% of G2/M cells, whereas almost none of the cells were in G2/M phase in control (FIG. 18A).

Treatment with a DQ or sCBD crude extract for 48 hours led to 64.14% or 84.27% cell apoptosis, respectively (FIG. 18B). Only 17.3% apoptosis was detected in the vehicle control. In addition, only low levels of necrosis were recorded with the sCBD treatment, not significantly different from the control (14.29 and 1.69%, respectively; FIG. 18B). However, both DQ extract and MMC led to relatively high levels of cell necrosis (35.30 and 25.13%, respectively).

Following, the effects of the extracts on cell migration was examined using scratch assays with treatments at IC50 concentrations of DQ (17.9911 g/mL) or sCBD (17.88 μg/mL), and compared to treatment with MMC (4 μg/mL) as a positive control. A marked and significant reduction in the ability of cells to migrate to the scratch was observed for cells treated with crude extracts of DQ or sCBD compared to the control (FIG. 19).

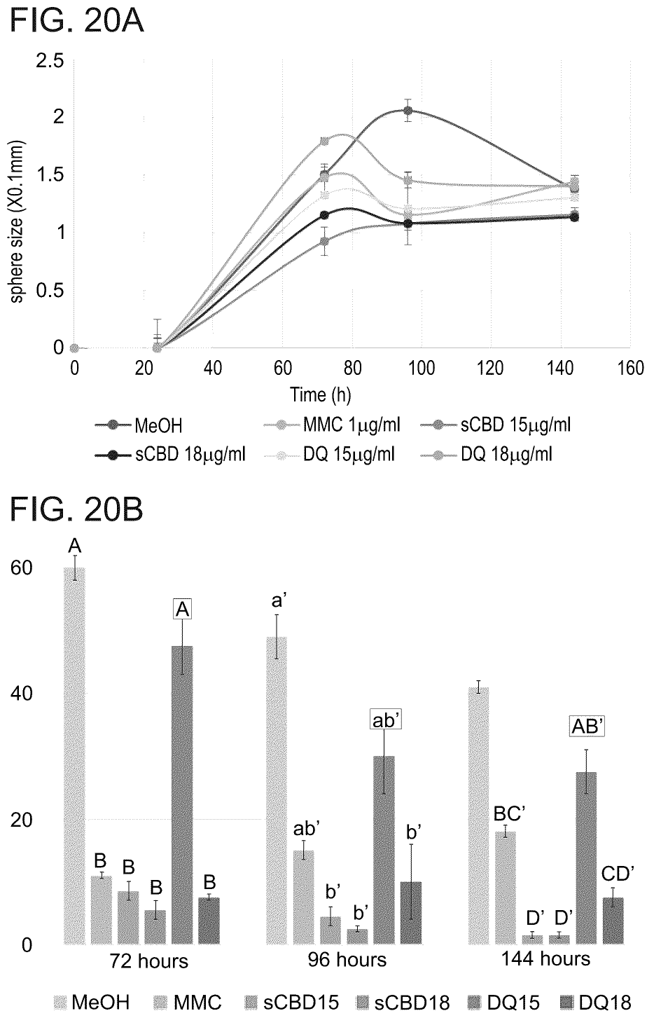

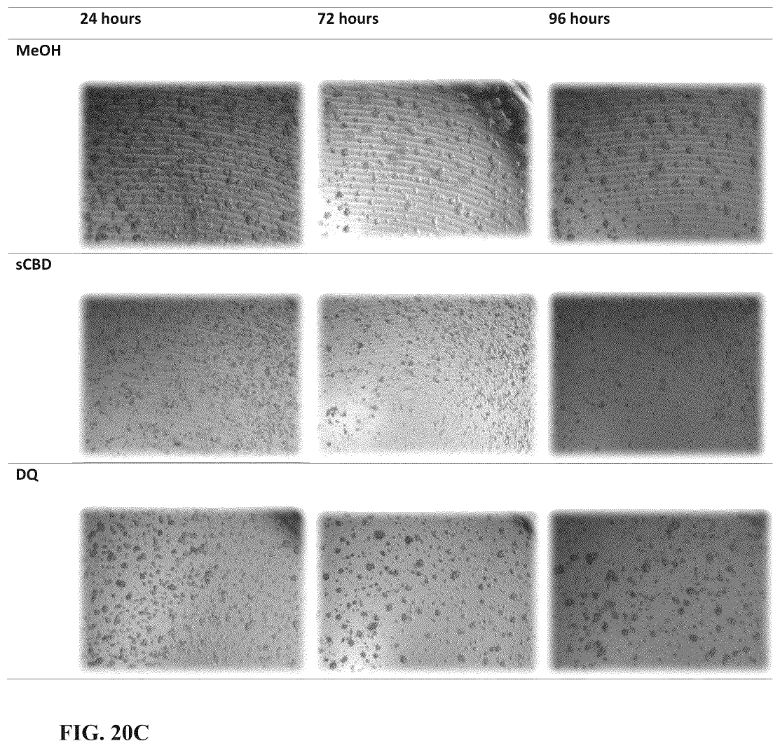

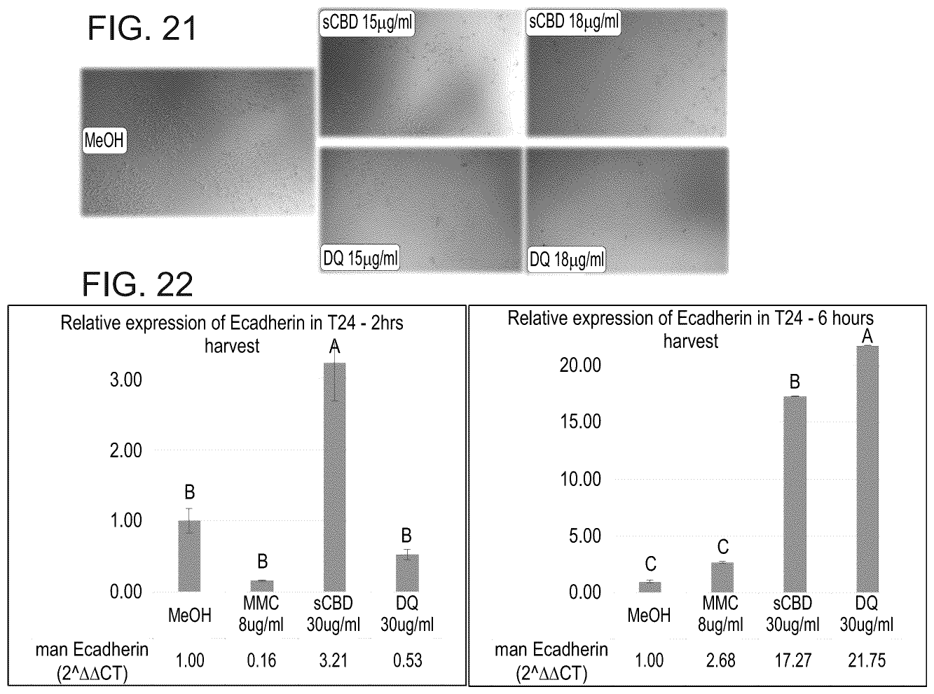

To assess the effect of the extracts on sphere formation, cells were exposed to treatment for 24 hours. Subsequently, a treatment washout was performed and the cells were incubated for another 24 hours. Only adherent cells were reseeded on a nonadherent plate, dead floating cells were removed. The formation of T24 spheres was evaluated 24 hours after treatments with sub-lethal concentrations of DQ (15 μg/mL and 18 μg/mL) or sCBD (15 μg/mL and 18 μg/mL) extract, or MMC (1 μg/mL). Treatment with a DQ or sCBD crude extract led to a change in cell adhesion appearance and inhibited sphere creation potentiality (FIGS. 20A-C). At 15 μg/mL, mean diameter of the sphere at 96 hours was reduced for sCBD or DQ compared to methanol treatment (0.108 mm, 0.121 mm and 0.205 mm, respectively). 18 μg/mL treatment completely inhibited the creation of spheres. The total sphere count demonstrated impaired sphere formation following treatment with 15 μg/mL sCBD or DQ compared to methanol treatment (FIGS. 20A-C). Following creation of the sphere, cells were transferred to normal culture conditions to assess the regeneration potential. The patches of colonies of UC cells were evaluated after 36 hours (2 cycles of generation time, defined as 19 hours by the manufacturer). Treatment with 18 μg/mL DQ or sCBD crude extract let to a profound inhibition of the regeneration potential, compared to vehicle control (FIG. 21).

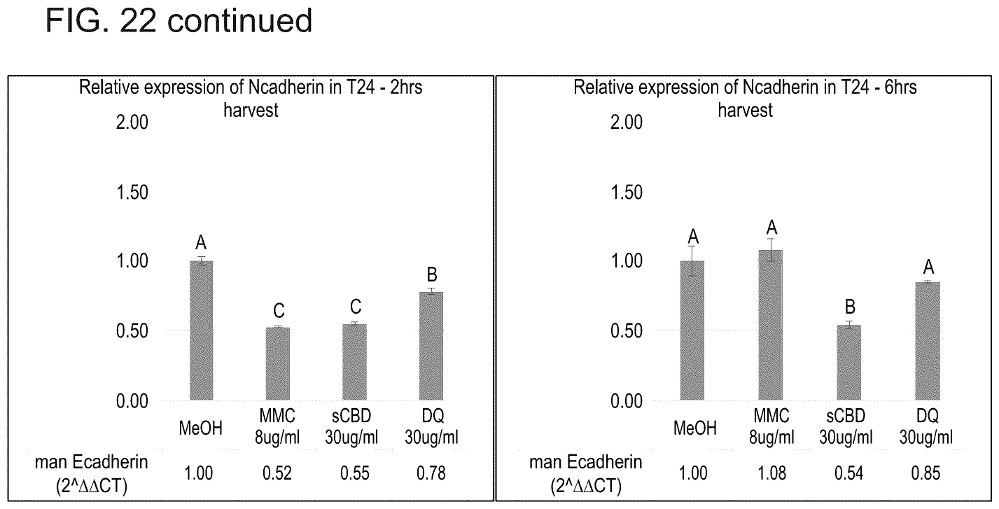

To assess the effect of treatments with the extracts on epithelial to mesenchymal transition potential, mRNA expression of E-cadherin and N-cadherin was determined. E-cadherin expression was significantly induced following 2 hours treatment with sCBD, when determined 2 hours or 6 hours after washout of the treatment (FIG. 22). E-cadherin expression was also significantly induced following 2 hours treatment with DQ crude extract or MMC when determined 6 hours after washout of treatment. However, its expression was reduced in these treatments when determined 2 hours after washout of the treatment (FIG. 22). N-cadherin expression was slightly reduced with MMC and sCBD crude extract treatments at 2 hours after washout of the treatments, and in sCBD treatment, a reduction in N-cadherin was also observed at 6 hours after washout of the treatment (FIG. 22).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Application

1. Thomas A. A., Wallner L. P. Quinn V. P., Slezak J., Van Den Eeden S. K., Chien G. W., Jacobsen S. J., Association between cannabis use and the risk of bladder cancer: results from the California Men's Health Study. Urology 2015, 85, 388-393.
2. Ramer R., Hinz B. Cannabinoids as anticancer drugs, in Advances in Pharmacology 80, ed. by D. Kendall, S. P. H. Alexander (Elsevier Academic Press, 2017). pp. 397-436.
3. Ramer R., Schwarz R., Hinz B. Modulation of the endocannabinoid system as a potential anticancer strategy. Front. Pharmacol. 2019, 10, 430.
4. Baram L., Peled E., Berman P., Yellin B., Besser E., Benami M., Louria-Hayon I., Lewitus G. M., Meiri D. The heterogeneity and complexity of Cannabis extracts as antitumor agents. Oncotarget 2019, 10, 4091.
5. Velasco G., Sánchez C., Guzmán M. Towards the use of cannabinoids as antitumour agents. Nat. Rev. Cancer 2012, 12, 436-444.
6. Cianchi F., Papucci L., Schiavone N., Lulli M., Magnelli L., Vinci M. C., Messerini L., Manera C., Ronconi E., Romagnani P. Cannabinoid receptor activation induces apoptosis through tumor necrosis factor α-mediated ceramide de novo synthesis in colon cancer cells. Clin. Cancer Res. 2008, 14, 7691-7700.
7. Galve-Roperh I., Sánchez C., Cortés M. L., del Pulgar T. G., Izquierdo M., Guzman M. Anti-tumoral action of cannabinoids: involvement of sustained ceramide accumulation and extracellular signal-regulated kinase activation. Nat. Med. 2000, 6, 313-319.
8. Bettiga A., Aureli M., Colciago G., Murdica V., Moschini M., Luciano R., Canals D., Hannun Y., Hedlund P., Lavorgna G. Bladder cancer cell growth and motility implicate cannabinoid 2 receptor-mediated modifications of sphingolipids metabolism. Sci. Rep. 2017, 7, 1-11.
9. Tyagi V., Philips B. J., Su R., Smaldone M. C., Erickson V. L., Chancellor M. B., Yoshimura N., Tyagi P. Differential expression of functional cannabinoid receptors in human bladder detrusor and urothelium. J. Urol. 2009, 181, 1932-1938.
10. Hanuš L. O., Meyer S. M., Muñoz E., Taglialatela-Scafati O., Appendino G. Phytocannabinoids: a unified critical inventory. Nat. Prod. Rep. 2016, 33, 1357-1392.
11. Berman P., Futoran K., Lewitus G. M., Mukha D., Benami M., Shlomi T., Meiri D. A new ESI-LC/MS approach for comprehensive metabolic profiling of phytocannabinoids in Cannabis. Sci. Rep. 2018, 8, 1-15.
12. Aizpurua-Olaizola O., Soydaner U., Öztürk E., Schibano D., Simsir Y., Navarro P., Etxebarria N., Usobiaga A. Evolution of the cannabinoid and terpene content during the growth of Cannabis sativa plants from different chemotypes. J. Nat. Prod. 2016, 79, 324-331.
13. Russo E. B. Taming THC: potential cannabis synergy and phytocannabinoid-terpenoid entourage effects. Br. J. Pharmacol. 2011, 163, 1344-1364.
14. Russo E. B. The Case for the entourage effect and conventional breeding of clinical cannabis: No “Strain,” No Gain. Front. Plant Sci. 2018, 9, 1969.
15. Koltai H., Namdar D. Cannabis phytomolecule ‘Entourage’: from domestication to medical use. Trends Plant Sci., 2020. doi: 10.1016/j.tplants.2020.04.007
16. Jeong S., Jo M. J., Yun H. K., Kim D. Y., Kim B. R., Kim J. L., Park S. H., Na Y. J., Jeong Y. A., Kim B. G. Cannabidiol promotes apoptosis via regulation of XIAP/Smac in gastric cancer. Cell Death Dis. 2019, 10, 1-13.
17. Velasco G., Hernández-Tiedra S., Dávila D., Lorente M. The use of cannabinoids as anticancer agents. Prog. Neuropsychopharmacol. Biol. Psychiatry 2016, 64, 259-266.
18. Velasco G., Sánchez C., Guzmán M. Anticancer mechanisms of cannabinoids. Curr. Oncol. 2016, 23, S23.
19. De Petrocellis L., Ligresti A., Schiano Moriello A., Iappelli M., Verde R., Stott C. G., Cristino L., Orlando P., Di Marzo V. Non-THC cannabinoids inhibit prostate carcinoma growth in vitro and in vivo: pro-apoptotic effects and underlying mechanisms. Br. J. Pharmacol. 2013, 168, 79-102.
20. Pollastro F., Caprioglio D., Del Prete D., Rogati F., Minassi A., Taglialatela-Scafati O., Munoz E., Appendino G. Cannabichromene. Nat. Prod. Commun. 2018, 13, 1934578X1801300922.
21. Pertwee R. G., Howlett A., Abood M. E., Alexander S., Di Marzo V., Elphick M., Greasley P., Hansen H. S., Kunos G., Mackie K. International Union of Basic and Clinical Pharmacology. LXXIX. Cannabinoid receptors and their ligands: beyond CB1 and CB2. Pharmacol. Rev. 2010, 62, 588-631.
22. Laprairie R., Bagher A., Kelly M., Denovan-Wright E. Cannabidiol is a negative allosteric modulator of the cannabinoid CB1 receptor. Br. J. Pharmacol. 2015, 172, 4790-4805.
23. Izzo A. A., Borrelli F., Capasso R., Di Marzo V., Mechoulam R. Non-psychotropic plant cannabinoids: new therapeutic opportunities from an ancient herb. Trends Pharmacol. Sci. 2009, 30, 515-527.
24. Andrade E. L., Forner S., Bento A. F., Leite D. F. P., Dias M. A., Leal P. C., Koepp J., Calixto J. B. TRPA1 receptor modulation attenuates bladder overactivity induced by spinal cord injury. Am. J. Physiol. Renal Physiol. 2011, 300, F1223-F1234.
25. Lazzeri M., Vannucchi M. G., Spinelli M., Bizzoco E., Beneforti P., Turini D., Faussone-Pellegrini M. S. Transient receptor potential vanilloid type 1 (TRPV1) expression changes from normal urothelium to transitional cell carcinoma of human bladder. Eur. Urol., 2005, 48, 691-698.
26. Yamada T., Ueda T., Shibata Y., Ikegami Y., Saito M., Ishida Y., Ugawa S., Kohri K., Shimada S. TRPV2 activation induces apoptotic cell death in human T24 bladder cancer cells: a potential therapeutic target for bladder cancer. Urology, 2010, 76, 509-el.
27. Muller C., Morales P., Reggio P. H. Cannabinoid ligands targeting TRP channels. Front. Mol. Neurosci. 2019, 11, 487.
28. Houtgraaf J. H., Versmissen J., van der Giessen W. J. A concise review of DNA damage checkpoints and repair in mammalian cells. Cardiovasc. Revasc. Med. 2006, 7, 165-172.
29. Kang S. G., Chung H., Yoo Y. D., Lee J. G., Choi Y. I., Yu Y. S. Mechanism of growth inhibitory effect of Mitomycin-C on cultured human retinal pigment epithelial cells: apoptosis and cell cycle arrest. Curr. Eye Res. 2001, 22, 174-181.
30. Olson M. F., Sahai E. The actin cytoskeleton in cancer cell motility. Clin. Exp. Metastasis 2009, 26, 273.
31. Wu L., Wang X., Liu Q., Leung A. W., Wang P., Xu C. Sinoporphyrin sodium mediated photodynamic therapy inhibits the migration associated with collapse of F-actin filaments cytoskeleton in MDA-MB-231 cells. Photodiagnosis Photodyn. Ther. 2016, 13, 58-65.
32. Pawlik A., Nowak J. M., Grzanka D., Gackowska L., Michalkiewicz J., Grzanka A. Hyperthermia induces cytoskeletal alterations and mitotic catastrophe in p53-deficient H1299 lung cancer cells. Acta Histochem. 2013, 115, 8-15.
33. Takahashi K., Suzuki K. WAVE2, N-WASP, and mena facilitate cell invasion via phosphatidylinositol 3-kinase-dependent local accumulation of actin filaments. J. Cell. Biochem. 2011, 112, 3421-3429.
34. Mazuz M., Moyal L., Hodak E., Nadarajan S., Vinayaka A. C., Gorovitz-Haris B., Lubin I., Drori A., Drori G., Van Cauwenberghe O., Faigenboim A., Namdar D., Amitay-Laish I., Koltai H. Synergistic cytotoxic activity of cannabinoids from Cannabis sativa against cutaneous T-cell lymphoma (CTCL) in-vitro and ex-vivo. Oncotarget 2020, 11, 1141.
35. Ye Z., Liang Z., Mi Q., Guo Y. Limonene terpenoid obstructs human bladder cancer cell (T24 cell line) growth by inducing cellular apoptosis, caspase activation, G2/M phase cell cycle arrest and stops cancer metastasis. Balkan Union of Oncology 2020, 25, 280-285.

1. (canceled)

2. A method of treating cancer in a subject in need thereof, the method comprising administering to the subject a therapeutically effective amount of a composition capable of reducing viability, inducing cell cycle arrest and/or reducing migration of cancer cells selected from the group consisting of:
(i) a composition comprising cannabinoids, wherein at least 80% of said cannabinoids comprise cannabichromene (CBC) or cannabichromene (CBC) and tetrahydrocannabinol (THC); and
(ii) a composition comprising cannabinoids, wherein at least 50% of said cannabinoids comprise cannabidiol (CBD) and/or tetrahydrocannabinol (THC), and at least one cannabinoid selected from the group consisting of cannabidivarinic acid (CBDVA), cannabigerol (CBG), tetrahydrocannabivarin (THCV), cannabinol (CBN), cannabichromene (CBC), cannabidiolic acid (CBDA), tetrahydrocannabinolic acid (THCA) and cannabidivarin (CBDV),
thereby treating the cancer in the subject.

3. A method of reducing viability, inducing cell cycle arrest and/or reducing migration of a cancerous cell, the method comprising contacting the cancerous cell with a composition capable of reducing viability, inducing cell cycle arrest and/or reducing migration of cancer cells selected from the group consisting of:
(i) a composition comprising cannabinoids, wherein at least 80% of said cannabinoids comprise cannabichromene (CBC) or at least 80% cannabichromene (CBC) and tetrahydrocannabinol (THC); and
(ii) a composition comprising cannabinoids, wherein at least 50% of said cannabinoids comprise cannabidiol (CBD) and/or tetrahydrocannabinol (THC), and at least one cannabinoid selected from the group consisting of cannabidivarinic acid (CBDVA), cannabigerol (CBG), tetrahydrocannabivarin (THCV), cannabinol (CBN), cannabichromene (CBC), cannabidiolic acid (CBDA), tetrahydrocannabinolic acid (THCA) and cannabidivarin (CBDV).

4. The method of claim 2, wherein said composition (i) comprises said CBC and said THC, wherein said composition comprises in said cannabinoids thereof at least 5% of said CBC and at least 5% of said THC.

5. A composition capable of reducing viability, inducing cell cycle arrest and/or reducing migration of cancer cells selected from the group consisting of:
(i) a composition comprising cannabinoids, wherein at least 80% of said cannabinoids comprise cannabichromene (CBC) and tetrahydrocannabinol (THC), wherein said composition comprises in said cannabinoids thereof at least 5% of said CBC and at least 5% of said THC; and
(ii) a composition comprising cannabinoids listed in the GB10011-F4 composition of Table 1 in percentages as listed in the GB10011-F4 composition of Table 1±10%, cannabinoids listed in the GB10011-F5 composition of Table 1 in percentages as listed in the GB10011-F5 composition of Table 1±10%, or cannabinoids listed in the GB10011-F6 composition of Table 1 in percentages as listed in the GB10011-F6 composition of Table 1±10%.

6. The method of claim 2, wherein said at least 80% in said composition (i) is at least 90%.

7. (canceled)

8. The method of claim 2, wherein said composition (i) comprises in said cannabinoids at least 10% of said CBC; and/or at least 10% of said THC.

9. (canceled)

10. The method of claim 2, wherein said composition (i) is devoid of a cannabinoid selected from the group consisting of cannabidiol (CBD), cannabigerol (CBG) and tetrahydrocannabinolic acid (THCA).

11. The method of claim 2, wherein said composition (i) is devoid of cannabinoids other than said CBC and THC.

12. The method of claim 2, wherein said composition (i) comprises said CBC and said THC in a concentration ratio of 15:1-1:15.

13. (canceled)

14. The method of claim 2, wherein said composition (i) comprises in said cannabinoids 80-85% CBC and 10-20% THC; or 20-30% CBC and 70-80% THC.

15. (canceled)

16. The method of claim 2, wherein said composition (i) has a combined synergistic cytotoxic activity on urothelial carcinoma cells as compared to each of said CBC and THC when administered as a single agent.

17-19. (canceled)

20. The method of claim 2, wherein said CBD is the most abundant cannabinoid in said composition (ii).

21. The method of claim 2, wherein said composition (ii):
(i) comprises the cannabinoids listed in the crude extract composition of Table 1 in percentages of cannabinoids as listed in the crude extract composition of Table 1±10%; or
(ii) comprises the cannabinoids listed in the GB10011-F4 composition of Table 1 in percentages of cannabinoids as listed in the GB10011-F4 composition of Table 1±10%; or
(iii) comprises the cannabinoids listed in the sCBD crude extract composition of Table 6 in percentages of cannabinoids as listed in the sCBD crude extract composition of Table 6±10%, and wherein said cancer is urothelial cancer; or
(iv) comprises the cannabinoids listed in the PARIS crude extract composition of Table 6 in percentages of cannabinoids as listed in the PARIS crude extract composition of Table 6±10%, and wherein said cancer is urothelial cancer; or
(v) comprises the cannabinoids listed in the GB10011-F5 composition of Table 1 in percentages of cannabinoids as listed in the GB10011-F5 composition of Table 1±10%; or
(vi) comprises the cannabinoids listed in the GB10011-F6 composition of Table 1 in percentages of cannabinoids as listed in the GB10011-F6 composition of Table 1±10%; or
(vii) comprises the cannabinoids listed in the DQ crude extract composition of Table 6 in percentages of cannabinoids as listed in the DQ crude extract composition of Table 6±10%, and wherein said cancer is urothelial cancer; or
(viii) comprises the cannabinoids listed in the DQ F7 composition of Table 6 in percentages of cannabinoids as listed in the DQ F7 composition of Table 6±10%, and wherein said cancer is urothelial cancer.

22-28. (canceled)

29. The method of claim 2, wherein said THC is the most abundant cannabinoid in said composition (ii).

30-37. (canceled)

38. The method of claim 2, wherein said composition is a synthetic composition.

39. The method of claim 2, wherein said composition (i) is a synthetic composition consisting of said CBC and said THC as the cannabinoids.

40. The method of claim 2, wherein said cancer is selected from the group consisting of urothelial cancer, uterine cervix carcinoma, prostate cancer and melanoma.

41. The method of claim 2, wherein said cancer is urothelial cancer.

42. (canceled)

43. The method of claim 2, further comprising administering to the subject a therapeutically effective amount of an anti-cancer agent.

44. (canceled)

45. An article of manufacture comprising the composition of claim 5 and an anti-cancer agent.

46-47. (canceled)

48. The method of claim 43, wherein said anti-cancer agent is selected from the group consisting of Mitomycin C, Cisplatinum, carboplatinum, MVAC (methotrexate, vinblastine, Adriamycin, cisplatin), gemzar and Cisplatin-Gemzar.

49-50. (canceled)
